(19) United States
(12) Patent Application Publication
(10) Pub. No.: US 2023/0407245 A1
(43) Pub. Date: Dec. 21, 2023

(54) Media and methods for establishing and maintaining early embryo-like cells

(71) Applicant: BGI SHENZHEN GROUP HOLDINGS CO., LIMITED, Shenzhen (CN)

(72) Inventors: Miguel A. Esteban, Guangzhou (CN); Md. Abdul Mazid, Guangzhou (CN); Wenjuan Li, Guangzhou (CN); Zhiwei Luo, Guangzhou (CN); Carl Ward, Guangzhou (CN); Yunpan Li, Guangzhou (CN); Yiwei Lai, Guangzhou (CN)

(21) Appl. No.: 18/036,578

(22) PCT Filed: Nov. 11, 2020
(86) PCT No.: PCT/CN2020/128091

§ 371 Date: May 11, 2023

(51) Int. Cl. C12N 5/00; C12N 5/0735; C12N 5/074

(52) U.S. Cl. CPC C12N 5/0031; C12N 5/0606; C12N 5/0696; C12N 2501/999; C12N 2500/38; C12N 2501/065; C12N 2501/235; C12N 2501/16; C12N 2500/12

(57) ABSTRACT

Provided are media and methods for establishing and maintaining mammalian early embryo-like cells. The culture media can be used to culture mammalian pluripotent stem cells (PSCs), are chemically defined, and comprise basal media for culturing stem cells supplemented with a S-adenosylhomocysteine hydrolase (SAH)/Polycomb repressive complexes (PRC)/EZH2 inhibitor and a histone deacetylase (HDAC) inhibitor. With the culture media thereof, primate (human and non-human) PSCs can be converted to preimplantation ICM-like cells (ICLCs) or 8-cell embryo-like cells (8CLCs).

### TECHNICAL FIELD

The subject invention relates to media and methods for establishing and maintaining mammalian early embryo-like cells.

### BACKGROUND

Mammalian embryogenesis is a complex process of cell division and differentiation that leads to development of an embryo. Successful fertilization of an oocyte with a sperm triggers the initiation of embryogenesis. This tightly controlled process gives rise to billions of cells with different functions and morphologies from a single zygote. The vast cellular complexity of all sexually reproducing organisms begins with embryogenesis. At the beginning, the single zygote will divide to form 2 cells. Then these 2 cells will subsequently divide to form 4 cells, 8 cells and 16 cells. After more expansion, the embryo becomes a blastocyst consisting of two regions, which are called inner cell mass (ICM) and trophectoderm (TE), respectively, until this stage embryonic development is termed preimplantation (in the uterine wall) stage. ICM cells will give rise to amnion and all fetal tissues, whereas the TE cells will give rise to the placenta during post-implantation stage of development. All these developmental stages have been well characterized in mice as we can easily access these cells from mouse embryos without ethical concerns. Seminal work by Evans and Kaufman showed that it was possible to extract cells from the ICM of mouse blastocysts and then grew these cells indefinitely in vitro under appropriate culture conditions (Evans and Kaufman, 1981). These cells are termed embryonic stem cells (ESCs) and are a representation of cells inside the ICM of mouse blastocyst. Mouse ESCs are pluripotent, but not totipotent, which means they can only differentiate into all three germ layers (ectoderm, mesoderm, and endoderm) of the embryo and therefore can generate cells corresponding to all fetal tissues. In contrast, totipotency is the ability of a cell to form a whole organism, including embryonic and extraembryonic cells, not just the fetal tissues as is the case for pluripotent cells. In mouse early embryos, cells from earlier than the 4-cell stage are totipotent, while in human, totipotency persists at least until the 8-cell (8C) stage (Hu, 2019). Seventeen years after Evans and Kaufman's discovery, Thomson and colleagues were able to generate human ESCs from human ICM (Thomson et al., 1998).

Due to the huge potential of human PSCs for disease modelling and regenerative medicine, much research was done to find an alternative source of these cells which would not require destruction of human embryos. In 2006, Takahashi and Yamanaka discovered a method to generate induced PSCs (iPSCs) exempt of ethical concerns from already differentiated cells (Takahashi and Yamanaka, 2006). ESCs and iPSCs are very similar and herein are referred collectively as PSCs.

Although both mouse ESCs and human ESCs are derived from the ICM of preimplantation blastocysts, they exhibit distinctive features. Human PSCs cultured in traditional conditions display a primed state of pluripotency which resembles mouse epiblast stem cells (EpiSCs) derived from post-implantation epiblasts (Brons et al., 2007; Tesar et al., 2007). Primed human PSCs display flat colony morphology, poor survival upon passaging as single cells, require fibroblast growth factor 2 (FGF2) and transforming growth factor-β1 (TGFβ1)/ACTIVIN A/NODAL signaling, and are unable to contribute to human-mouse interspecies chimera formation. In contrast, mouse ESCs reside in a naïve state closer to preimplantation ICM which is characterized by dome-shape colonies, increased single-cell clonogenicity, dependence on Janus kinase/signal transducer and activator of transcription 3 (JAK/STAT3) signaling, a preimplantation ICM-like transcriptome profile and capability of chimerism (Nichols and Smith, 2011; Ying et al., 2008). Moreover, mouse ESCs have a greater differentiation potential than EpiSCs (Honda et al., 2013). In addition, it has been reported in recent years that a small population of cells (˜0.5%) in mouse ESC cultures display a transcriptional profile similar to that of the 2-cell (2C) stage mouse embryo (Macfarlan et al., 2012). This is important because 2C cells are totipotent.

Recently, multiple methods have been published to derive and maintain altered states of human and non-human primate PSCs (Gafni et al., 2013; Takashima et al., 2014; Theunissen et al., 2014) that exhibit human naïve (preimplantation-like) characteristics. These cells share some morphological and molecular similarities with mouse ESCs. However, whether these reported human naïve PSCs are truly similar to the preimplantation ICM is under debate. Moreover, each of the current methods has specific drawbacks, such as being lengthy, producing variable levels of naïve specific genes, transgene dependency for naïve induction, genomic instability, imprinting loss, inability of multi-lineage differentiation, and inefficient or no proper chimera formation competency.

PSCs have great potential to be used for cell therapy in regenerative medicine and to study disease through patient specific disease modelling (Shi et al., 2017). Currently researchers are using primed PSCs as source material for these studies. One area where naïve cells are starting to prove useful is in generation of inter-species chimeras. These experiments involve injecting PSCs of one species into a developing embryo of another species and then measuring the percentage of cells that contribute to the organism. However, the contribution to the chimeras is currently exceptionally low (<0.01%). We believe that PSCs closer resembling transcriptionally and epigenetically to the early embryo would improve chimera contribution and indeed PSC function as a whole.

Another exciting area of research with PSCs is blastoid formation. Blastoids are blastocyst-like structures which are currently formed in vitro by forced aggregation of ESCs and TE cells (Shahbazi and Zernicka-Goetz, 2018). These in vitro models of the early embryo will shed new light on the developmental process and could be used to model diseases which affect embryogenesis. Nevertheless, the current state-of-the-art models requires mixing several types of cells, rather than all the cells arising from a single cell and self-organizing, and the blastoids fail to behave like real blastocysts, for example, they cannot gastrulate properly (Li et al., 2019). We believe that using cells which are closer resembling transcriptionally and epigenetically to the early embryo will improve this process and could result in the formation of bona fide blastoids.

The major controller of cell fate transition during development is epigenetic. This implies that manipulating the epigenome, we should be able to produce cells matching any developmental stage. One of the best examples of this exploitation is the generation of iPSCs from somatic cells. Here, transient expression of transcription factors or chemical compounds is enough to turn fully differentiated cells into PSCs (Hou et al., 2013; Takahashi and Yamanaka, 2006). Other examples include the above-mentioned conversion of primed state PSCs to a naïve state using small molecule inhibitors of epigenetic pathways and cytokines. One of the key constituents of the epigenome is DNA methylation, which plays a central role in gene regulation. The overall DNA methylation content of cells in early embryogenesis is highly dynamic. It is known that DNA methylation of preimplantation blastocyst is much lower than post-implantation embryo, and, interestingly, also lower than the 8C embryo (Zhu et al., 2018). Therefore, reversion of primed PSCs to an ICM-like state requires a significant reduction of the overall DNA methylation level, whereas, accordingly, for capturing an 8C-like stage would require a more controlled reduction. In addition, DNA methylation landscape needs to be rewired correctly over the reversion process, respecting, imprinting control regions (ICR) should be maintained hemimethylated. Therefore, fine-tuning of DNA methylation machinery is strictly necessary for producing early embryo-like cells.

### SUMMARY OF THE INVENTION

In one aspect, the present disclosure discloses a chemically defined culture medium for culturing PSCs comprising a basal medium for culturing stem cells supplemented with a Polycomb repressive complexes (PRC) and/or EZH2 inhibitor and a histone deacetylase (HDAC) inhibitor.

In one or more embodiments, the PRC and/or EZH2 inhibitor is a S-adenosylhomocysteine hydrolase (SAH) inhibitor.

In one or more embodiments, the chemically defined culture medium is further supplemented with one or more components selected from a group consisting of L-ascorbic acid or a derivative thereof, an activator of JAK/STAT3 signaling, an inhibitor of mitogen-activated protein kinases/extracellular signal-regulated kinases (MAPK/ERK) signaling, and a tankyrase inhibitor; optionally, the culture medium is further supplemented with one or more components selected from a group consisting of an activator of ACTIVIN/NODAL signaling, a Rho-associated protein kinases (ROCK) inhibitor, and an extracellular matrix.

In one or more embodiments, the PRC/EZH2 inhibitor is selected from a group consisting of 3-deazaneplanocin A (DZNep) and CPI-1205.

In one or more embodiments, DZNep is present in the culture medium at a final concentration of 5 to 80 nM, preferably 5 to 50 nM.

In one or more embodiments, CPI-1205 is present in the culture medium at a final concentration of 0.5 to 5 mM, preferably 1 to 3 mM.

In one or more embodiments, the HDAC inhibitor is selected from a group consisting of trichostatin A (TSA), valproic acid (VPA) and sodium butyrate (NaB).

In one or more embodiments, TSA is present in the culture medium at a final concentration of 3 to 30 nM, preferably 3 to 25 nM.

In one or more embodiments, VPA is present in the culture medium at a final concentration of 0.25 to 2 mM, preferably 0.5 to 1.5 mM.

In one or more embodiments, NaB is present in the culture medium at a final concentration of 0.25 to 2 mM, preferably 0.5 to 1.5 mM.

In one or more embodiments, the final concentration of L-ascorbic acid in the culture medium is 40 to 70 ng/mL.

In one or more embodiments, the final concentration of the activator of JAK/STAT3 signaling in the culture medium is 10 to 50 ng/mL.

In one or more embodiments, the activator of JAK/STAT3 signaling is LIF.

In one or more embodiments, the final concentration of PD0325901 in the culture medium is 0.5 to 3 μM.

In one or more embodiments, the inhibitor of MAPK/ERK signaling is PD0325901.

In one or more embodiments, the final concentration of the tankyrase inhibitor in the culture medium is 2 to 8 μM.

In one or more embodiments, the tankyrase inhibitor is selected from a group consisting of IWR1 and XAV939.

In one or more embodiments, the final concentration of the activator of ACTIVIN/NODAL signaling is from 10 to 25 ng/mL.

In one or more embodiments, the activator of ACTIVIN/NODAL signaling is selected from a group consisting of ACTIVIN A and NODAL.

In one or more embodiments, the final concentration of the ROCK inhibitor in the culture medium is 0.5 to 2 μM.

In one or more embodiments, the ROCK inhibitor is selected from a group consisting of Y27632, thiazovivin and hydroxyfasudil.

In one or more embodiments, the amount of the extracellular matrix in the culture medium is 0.1 to 0.5% (v/v).

In one or more embodiments, the extracellular matrix is selected from a group consisting of Matrigel™, Geltrex™ and ECM™.

In one or more embodiments, the culture medium comprises DZNep at a final concentration of 5 to 15 nM or CPI-1205 at a final concentration of 0.5 to 3 mM; TSA at a final concentration of 3 to 10 nM, or VPA at a final concentration of 0.25 to 1 mM or NaB at a final concentration of to 1 mM; L-ascorbic acid at a final concentration of 40 to 70 μg/mL; LIF at a final concentration of 10 to 30 ng/mL; PD0325901 at a final concentration of 0.5 to 1.5 μM; and IWR1 or XAV939 at a final concentration of 3 to 6 μM; and is further supplemented with:
(1) ACTIVIN A or NODAL at a final concentration of 10 to 25 ng/mL; Y27632, thiazovivin or hydroxyfasudil at a final concentration of 0.5 to 2 μM; and an extracellular matrix in an amount of 0.1% to 0.5% (v/v); or
(2) ACTIVIN A or NODAL at a final concentration of 10 to 25 ng/mL; and Y27632, thiazovivin or hydroxyfasudil at a final concentration of 0.5 to 2 μM; or
(3) ACTIVIN A or NODAL at a final concentration of 10 to 25 ng/mL; and an extracellular matrix in an amount of 0.1% to 0.5% (v/v); or
(4) Y27632, thiazovivin or hydroxyfasudil at a final concentration of 0.5 to 2 μM; and an extracellular matrix in an amount of 0.1% to 0.5% (v/v); or
(5) ACTIVIN A or NODAL at a final concentration of 10 to 25 ng/mL; or Y27632, thiazovivin or hydroxyfasudil at a final concentration of 0.5 to 2 μM; or an extracellular matrix in an amount of 0.1% to 0.5% (v/v).

In one or more embodiments, the culture medium comprises 10 nM of DZNep or 1 mM of CPI-1205; 5 nM of TSA, or 0.5 mM of VPA, or 0.5 mM of NaB; 50 μg/mL of L-ascorbic acid; 20 ng/mL of LIF; 1 μM of PD0325901; and 5 μM of IWR1 or 5 μM of XAV939; and is further supplemented with (1) 20 ng/mL of human ACTIVIN A or human NODAL, 1 μM of Y27632, thiazovivin or hydroxyfasudil, and 0.2% (v/v) of an extracellular matrix; or (2) 20 ng/mL of ACTIVIN A or NODAL, and 1 μM of Y27632, thiazovivin or hydroxyfasudil; (3) 20 ng/mL of ACTIVIN A or NODAL, and 0.2% (v/v) of an extracellular matrix; or (4) 1 μM of Y27632, thiazovivin or hydroxyfasudil, and 0.2% (v/v) of an extracellular matrix; or (5) 20 ng/mL of ACTIVIN A or NODAL, or 1 μM of Y27632, thiazovivin or hydroxyfasudil, or 0.2% (v/v) of an extracellular matrix.

In one or more embodiments, the culture medium comprises DZNep at a final concentration of 40 to 70 nM or CPI-1205 at a final concentration of 2 to 4 mM; TSA at a final concentration of 10 to 30 nM, or VPA at a final concentration of 0.5 to 1.5 mM or NaB at a final concentration of 0.5 to 1.5 mM; L-ascorbic acid at a final concentration of 40 to 70 μg/mL; LIF at a final concentration of 10 to 30 ng/mL; PD0325901 at a final concentration of 0.5 to 1.5 μM; and IWR1 or XAV939 at a final concentration of 3 to 6 μM; and is further supplemented with:
(1) ACTIVIN A or NODAL at a final concentration of 10 to 25 ng/mL; Y27632, thiazovivin or hydroxyfasudil at a final concentration in a range 0.5 to 2 μM; and an extracellular matrix in an amount of 0.1% to 0.5% (v/v); or
(2) ACTIVIN A or NODAL at a final concentration of 10 to 25 ng/mL; and Y27632, thiazovivin or hydroxyfasudil at a final concentration in a range 0.5 to 2 μM; or
(3) ACTIVIN A or NODAL at a final concentration of 10 to 25 ng/mL; and an extracellular matrix in an amount of 0.1% to 0.5% (v/v); or
(4) Y27632, thiazovivin or hydroxyfasudil at a final concentration in a range 0.5 to 2 μM; and an extracellular matrix in an amount of 0.1% to 0.5% (v/v); or
(5) ACTIVIN A or NODAL at a final concentration of 10 to 25 ng/mL; or Y27632, thiazovivin or hydroxyfasudil at a final concentration in a range 0.5 to 2 μM; or an extracellular matrix in an amount of 0.1% to 0.5% (v/v).

In one or more embodiments, the culture medium comprises 50 nM DZNep or 3 mM CPI-1205; 20 nM TSA, or 1 mM VPA, or 1 mM NaB; 50 μg/mL L-ascorbic acid; 20 ng/mL LIF; 1 μM PD0325901; and 5 μM IWR1 or 5 μM XAV939; and is further supplemented with (1) 20 ng/mL of ACTIVIN A or NODAL, 1 μM of Y27632, thiazovivin or hydroxyfasudil, and 0.2% (v/v) of an extracellular matrix; or (2) 20 ng/mL of ACTIVIN A or NODAL, and 1 μM of Y27632, thiazovivin or hydroxyfasudil; (3) 20 ng/mL of ACTIVIN A or NODAL, and 0.2% (v/v) of an extracellular matrix; or (4) 1 μM of Y27632, thiazovivin or hydroxyfasudil, and 0.2% (v/v) of an extracellular matrix; or (5) 20 ng/mL of ACTIVIN A or NODAL, or 1 μM of Y27632, thiazovivin or hydroxyfasudil, or 0.2% (v/v) of an extracellular matrix.

In one or more embodiments, the basal medium is selected from a group consisting of Dulbecco's modified eagle's medium (DMEM), minimal essential medium (MEM), basal medium Eagle (BME), RPMI1640, F10, F12, α minimal essential medium (a MEM), Glasgow's minimal essential medium (GMEM), Iscove's modified Dulbecco's medium, Neurobasal Medium, DMEM/F12 and Advanced DMEM/F12 and a combination thereof; preferably, the basal medium is a mixture of Advanced DMEM/F12 and Neurobasal Medium in a ratio of 1:1 (v/v).

In one or more embodiments, the culture medium is further supplemented with one or more components selected from a group consisting of serum replacement, alternative carbon source, non-essential amino acid, L-glutamine or its alternative and antibiotic.

In one or more embodiments, the serum replacement is selected from a group consisting of Knockout™ Serum Replacement (KOSR), N2 and B27, and combinations thereof; preferably, the serum replacement is a mixture of N2 and B27 in a ratio of 1:1 (w/w); the alternative carbon source is pyruvate, such as sodium pyruvate; the L-glutamine or its alternative is Glutamax™ supplement comprising L-alanyl-L-glutamine dipeptide in 0.85% NaCl; and/or the antibiotic is selected from a group consisting of penicillin, streptomycin, or a mixture of penicillin and streptomycin.

In another aspect, the present disclosure discloses a method for converting primate PSCs to preimplantation ICM-like cells (ICLCs) and/or 8-cell embryo-like cells (8CLCs), comprising culturing the primate PSCs or the ICLCs in the presence of a SAH/PRC/EZH2 inhibitor and a HDAC inhibitor. The present disclosure further discloses a method for converting ICLCs to 8CLCs, comprising culturing the ICLCs in the presence of a SAH/PRC/EZH2 inhibitor and a HDAC inhibitor.

In one or more embodiments, the method comprises culturing the primate PSCs or the ICLCs in the presence of a SAH/PRC/EZH2 inhibitor and a HDAC inhibitor, and one or more components selected from a group consisting of L-ascorbic acid, an activator of JAK/STAT3 signaling, an inhibitor of MAPK/ERK signaling and a tankyrase inhibitor, and optionally in the presence of one or more components selected from a group consisting of an activator of ACTIVIN/NODAL signaling, a ROCK inhibitor, and an extracellular matrix.

In one or more embodiments, the SAH/PRC/EZH2 inhibitor is selected from a group consisting of DZNep and CPI-1205.

In one or more embodiments, the HDAC inhibitor is selected from a group consisting of TSA, VPA and NaB.

In one or more embodiments, the primate PSCs or the ICLCs are cultured in the presence of DZNep at a final concentration of 5 to 80 nM, preferably 5 to 50 nM or CPI-1205 at a final concentration of 0.5 to 5 mM, preferably 1 to 3 mM, and in the presence of TSA at a final concentration of 3 to 30 nM, preferably 3 to 25 nM, or VPA at a final concentration of 0.25 to 2 mM, preferably 0.5 to 1.5 mM, or NaB at a final concentration of 0.25 to 2 mM, preferably 0.5 to 1.5 mM.

In one or more embodiments, L-ascorbic acid is present at a final concentration of 40 to 70 μg/mL.

In one or more embodiments, the final concentration of the activator of JAK/STAT3 signaling is 10 to 50 ng/mL.

In one or more embodiments, the activator of JAK/STAT3 signaling is LIF.

In one or more embodiments, the final concentration of the inhibitor of MAPK/ERK signaling is 0.5 to 3 μM.

In one or more embodiments, the inhibitor of MAPK/ERK signaling is PD0325901.

In one or more embodiments, the final concentration of the tankyrase inhibitor is 2 to 8 μM.

In one or more embodiments, the tankyrase inhibitor is selected from a group consisting of IWR1 and XAV939.

In one or more embodiments, the final concentration of the activator of ACTIVIN/NODAL signaling is from 10 to 25 ng/mL.

In one or more embodiments, the activator of ACTIVIN/NODAL signaling is selected from a group consisting of human ACTIVIN A and human NODAL.

In one or more embodiments, the final concentration of the ROCK inhibitor is 0.5 to 2 μM.

In one or more embodiments, the ROCK inhibitor is selected from a group consisting of Y27632, thiazovivin and hydroxyfasudil.

In one or more embodiments, the extracellular matrix is present at an amount of 0.1-0.5% (v/v).

In one or more embodiments, the extracellular matrix is selected from a group consisting of Matrigel™, Geltrex™ and ECM™.

In another aspect, the present disclosure further discloses a method for converting primate PSCs to ICLCs, comprising culturing the primate PSCs in a culture medium of present disclosure for converting to ICLCs, wherein the basal medium of the culture medium is selected from a group consisting of Dulbecco's modified eagle's medium (DMEM), minimal essential medium (MEM), basal medium Eagle (BME), RPMI1640, F10, F12, a minimal essential medium (a MEM), Glasgow's minimal essential medium (GMEM), Iscove's modified Dulbecco's medium, Neurobasal Medium and DMEM/F12, and a combination thereof; preferably, the basal medium is a mixture of Advanced DMEM/F12 and Neurobasal Medium in a ratio of 1:1 (v/v).

In a further aspect, the present disclosure discloses a method for converting primate PSCs or ICLCs to 8CLCs, comprising culturing the primate PSCs or ICLCs in the culture medium of present disclosure for converting primate PSCs to ICLCs or 8CLCs, wherein the basal medium of the culture medium is selected from a group consisting of Dulbecco's modified eagle's medium (DMEM), minimal essential medium (MEM), basal medium Eagle (BME), RPMI1640, F10, F12, a minimal essential medium (a MEM), Glasgow's minimal essential medium (GMEM), Iscove's modified Dulbecco's medium, Neurobasal Medium, DMEM/F12 and Advanced DMEM/F12, and a combination thereof; preferably, the basal medium is a mixture of Advanced DMEM/F12 and Neurobasal Medium in a ratio of 1:1 (v/v).

In one or more embodiments, the primate PSCs are selected from a group consisting of:
(i) cells from an ESC line and/or an ECC line;
(ii) cells from an iPSC line;
(iii) cells from ICM of a preimplantation blastocyst cultured in vitro;
(iv) cells from ICM of a post-implantation blastocyst cultured in vitro;
(v) cells from an embryo of 8C stage to morula stage cultured in vitro.

In one or more embodiments, the primate PSCs or the ICLCs are cultured under one or more conditions selected from a group consisting of: (i) on feeder cells; (ii) on an extracellular matrix devoid of feeders; (iii) in suspension devoid of feeder cells; (iv) propagation in hypoxic or normoxic condition at about 37° C. temperature; (v) passaging as single cells every 3 to 4 days with a split ratio of 1:4 to 1:8; (vi) changing medium daily.

In a further aspect, the present disclosure provides an isolated ICLC having transcriptome, transposable elements profile, DNA methylome, chromatin landscape, and metabolic state close to a corresponding primate preimplantation ICM.

In one or more embodiments, the primate ICLCs are further characterized by one or more of the following characteristics:
1) being able to self-renew and maintain pluripotency in culture;
2) maintaining genomic stability in culture according to karyotype;
3) being able to give rise to cells of the 3 germ layers;
4) being able to give rise to primordial germ cell-like cells;
being able to integrate to mouse embryo and contribute to embryonic and extraembryonic tissues;
6) being able to transit to extraembryonic cell fate in vitro; and
7) being able to form blastocyst-like structures in vitro.

In one or more embodiments, the ICLCs is obtained by any of the methods described in the present application for producing ICLCs.

In a further aspect, the present disclosure provides an isolated primate 8CLC expressing 8C embryo specific markers at a level substantially higher than ICLCs and/or primed PSCs; preferably, the cells have transcriptome, transposable element profile and chromatin landscape close to corresponding primate 8C stage embryos.

In one or more embodiments, the 8CLCs are further characterized by one or more of the following characteristics:
1) maintaining genomic stability in culture according to karyotype;
2) being able to give rise to cells of the 3 germ layers;
3) being able to give rise to primordial germ cell-like cells;
4) being able to integrate to mouse embryos and contribute to embryonic and extraembryonic tissues;
5) being able to transit to extraembryonic cell fate in vitro; and
6) being able to form blastocyst-like structures in vitro.

In one or more embodiments, the 8CLCs is obtained by any of the methods described in the present application for producing 8CLCs.

The present disclosure also provides a cell culture containing the primate ICLCs and/or the 8CLCs as described in any of the embodiments of the present application, and a culture medium; preferably, the culture medium is defined in any of the culture medium embodiments of the present application.

The present disclosure also provides a kit comprising a SAH/PRC/EZH2 inhibitor and a HDAC inhibitor, and optionally
(1) one or more components selected from a group consisting of L-ascorbic acid, an activator of JAK/STAT3 signaling, an inhibitor of MAPK/ERK signaling, and a tankyrase inhibitor;
(2) one or more components selected from a group consisting of an activator of ACTIVIN/NODAL signaling, a ROCK inhibitor and an extracellular matrix;
(3) one or more components selected from a group consisting of basal culture medium, serum replacement, alternative carbon source, non-essential amino acid, L-glutamine or its alternative and antibiotic.

In one or more embodiments, the kit comprises the culture medium as defined in any of the culture medium embodiments of the present application.

The present disclosure also provides a composition comprising a SAH/PRC/EZH2 inhibitor and a HDAC inhibitor, and optionally
(1) one or more components selected from a group consisting of L-ascorbic acid, an activator of JAK/STAT3 signaling, an inhibitor of MAPK/ERK signaling, and a tankyrase inhibitor; and
(2) one or more components selected from a group consisting of an activator of ACTIVIN/NODAL signaling and a ROCK inhibitor.

In one or more embodiments, the composition comprises DZNep or CPI-1205, and TSA or VPA or NaB, and optional L-ascorbic acid, optional LIF, optional PD0325901 and optional IWR1 or XAV939; preferably, each of the components is present in an amount that allows the culture medium containing the composition to comprise: 5 to 15 nM, preferably 10 nM, of DZNep, or 0.5 to 2 mM, preferably 1 mM, of CPI-1205; 3 to 6 nM, preferably 5 nM, of TSA, or 0.25 to 1 mM, preferably 0.5 mM, of VPA, or 0.25 to 1 mM, preferably 0.5 mM, of NaB; and optionally 40 to 90 μg/mL, preferably 50 μg/mL, of L-ascorbic acid, optionally 10 to 30 ng/mL, preferably 20 ng/mL, of LIF, optionally 0.5 to 1.5 μM, preferably 1 μM, of PD0325901, optionally 3 to 6 μM, preferably 5 μM, of IWR1 or XAV939. The compositions may further comprise ACTIVIN A or NODAL, and/or Y27632, thiazovivin or hydroxyfasudil, and/or an extracellular matrix, wherein each of the components is present in an amount that allows the culture medium containing the composition to comprise 10 to 25 ng/mL, preferably 20 ng/mL ACTIVIN A or NODAL, and/or 0.5 to 2 μM, preferably 1 μM, of Y27632, thiazovivin or hydroxyfasudil, and/or 0.1% to 0.5% (v/v) of an extracellular matrix.

In one or more embodiments, the composition comprises DZNep or CPI-1205, and TSA or VPA or NaB, and optional L-ascorbic acid, optional LIF, optional PD0325901 and optional IWR1 or XAV939; preferably, each of the components is present in an amount that allows the culture medium containing the composition to comprise: 40 to 70 nM, preferably 50 nM, of DZNep, or 2 to 4 mM, preferably 3 mM, of CPI-1205; 10 to 30 nM, preferably 20 nM, of TSA, or 0.5 to 1.5 mM, preferably 1 mM, of VPA, or 0.5 to 1.5 mM, preferably 1 mM, of NaB; and optionally 40 to 90 μg/mL, preferably 50 μg/mL, of L-ascorbic acid, optionally 10 to 30 ng/mL, preferably 20 ng/mL, of LIF, optionally 0.5 to 1.5 μM, preferably 1 μM, of PD0325901, optionally 3 to 6 μM, preferably 5 μM, of IWR1 or XAV939. The compositions may further comprises ACTIVIN A or NODAL, and/or Y27632, thiazovivin or hydroxyfasudil, and/or an extracellular matrix, wherein each of the components is present in an amount that allows the culture medium containing the composition to comprise 10 to 25 ng/mL, preferably 20 ng/mL ACTIVIN A or NODAL, and/or 0.5 to 2 μM, preferably 1 μM, of Y27632, thiazovivin or hydroxyfasudil, and/or 0.1% to 0.5% (v/v) of an extracellular matrix.

The present disclosure also provides use of an agent which can promote expression of STELLA (also named DPPA3 and PGC7) or improve activity of STELLA in the manufacture of a reagent, a culture medium or a kit for promoting conversion of primate PSCs to ICLCs, or for promoting conversion of primate PSCs or ICLCs to 8CLCs, and use of an agent which can promote expression of STELLA or improve activity of STELLA for promoting conversion of primate PSCs to ICLCs, or for promoting conversion of primate PSCs or ICLCs to 8CLCs.

In one or more embodiments, the agent which can promote expression of STELLA or improve activity of STELLA is an inhibitor of SAH/PRC/EZH2, which includes but is not limited to DZNep and CPI-1205. Preferably, the inhibitor of SAH/PRC/EZH2, such as DZNep and CPI-1205, is used in the above use in an amount as described in any of the embodiments described in the subject application.

The present disclosure further provides use of an agent which can promote expression of KHDC1L, TRIM60, and/or genes belonging to eutherian totipotent cell homeobox (ETCHbox) family including TPRX1 and ARGFX, or improve activity of KHDC1L, TRIM60, and/or proteins belonging to ETCHbox family including TPRX1 and ARGFX, in the manufacture of a reagent, a culture medium or a kit for promoting conversion of primate PSCs or ICLCs to 8CLCs, and use of an agent which can promote expression of KHDC1L, TRIM60, and/or genes belonging to ETCHbox family including TPRX1 and ARGFX, or improve activity of KHDC1L, TRIM60, and/or proteins belonging to ETCHbox family including TPRX1 and ARGFX for promoting conversion of primate PSCs or ICLCs to 8CLCs.

In one or more embodiments, the agent which can promote expression of TPRX1, KHDC1L, and/or TRIM60, or improve activity of TPRX1, KHDC1L, and/or TRIM60 is an inhibitor of SAH/PRC/EZH2, which includes but is not limited to DZNep and CPI-1205. Preferably, the inhibitor of SAH/PRC/EZH2, such as DZNep and CPI-1205, is used in the above use in an amount as described in any of the embodiments described in the subject application.

### DESCRIPTION OF DRAWINGS

FIG. 1. (A) Schematic representing the protocol used by the inventors to generate human ICLCs. Briefly, primed human PSCs which were cultured in mTeSR media were changed into ICLC conversion media (4CL) and were grown for 12 days, and cells were passaged at day 4 and 8. (B) Phase contrast microscope images showing the morphology of primed human PSCs (left panel) and ICLCs converted by 4CL medium 1 (right panel). (C) Representative immunofluorescence microscope images of ICLCs colonies. Nuclei were stained with anti-KLF17 (upper row, middle column), anti-NANOG (middle row, middle column) or anti-OCT4 (lower row, middle column), and counter stained with DAPI (left column). Different channels were merged (right column).

FIG. 2. (A) 2D scatter plot showing UMAP transformed single-cell RNA-seq gene expression of H9 cells at the primed stage (day 0) and then at day 1, 2, 3, 5, 8 and 12 after being cultured in the 4CL medium 1. The inventors also included published single-cell RNA-seq data from human embryo cells of embryonic day 3 (E3), 4 (E4), 5 (E5), 6 (E6) and 7 (E7) (from E-MTAB-3929). (B) Heatmap illustrating the expression levels of known naïve markers in primed H9, 4CL medium 1 converted H9 and human ICM cells (from GSE101571) (right panel).

FIG. 3. (A) 2D scatter plot showing UMAP transformed single-cell RNA-seq transposable element (TE) expression of H9 cells at the primed stage (day 0) and then at day 1, 2, 3, 5, 8 and 12 after being cultured in the 4CL medium 1. The inventors also included published single-cell RNA-seq TE expression data from human embryo cells of embryonic day 3, 4, 5, 6 and 7 (from E-MTAB-3929). (B) Heatmap illustrating the expression levels of known naïve TEs in primed H9, 4CL medium 1 converted H9 and human ICM cells (from GSE101571).

FIG. 4. Representative images of chromosomes after Giemsa staining to illustrate that the cells maintained stable karyotype over long term culture. Karyotyping was carried out on primed H9 (upper left panel), 4CL medium 1 converted H9 at passage 15 (upper right panel), primed UH10 (lower left panel) and 4CL medium 1 converted UH10 at passage 15 (lower right panel).

FIG. 5. Box plots showing the CpG methylation levels across the whole genome (left column), and at 2 kb around the TSS of all genes (right column).

FIG. 6. Heatmap showing the CpG methylation levels at selected ICRs in human ICM cells compared to 4CL converted cells.

FIG. 7. (A-D) 2D scatter plot showing a UMAP visualization of chromatin accessibility at KLF17, DPPA3/STELLA, DPPA5, CD70, POU5F1, and THY1 loci in primed and ICLC single-cells.

FIG. 8. (A) Differentially accessible chromatin regions in primed human PSCs and cells in the process of conversion to ICLCs using 4CL medium 1. Regions which are close in primed become open during conversion to ICLCs (upper panel). Regions which are open in primed turn to close during conversion to ICLCs (lower panel). (B) Motif enrichment analysis showing a selection of motifs that were enriched in the close to open regions (upper) and the open to close regions (lower) during primed to ICLC conversion. (C) Bar plot showing expression levels of TFAP2C, KLF5, SOX3 and ZIC3 in primed human PSCs and ICLCs after 12 days of conversion.

FIG. 9. (A) Bar chart showing the elevation of oxidative phosphorylation (OxPhos) related genes in 4CL medium 1 converted ICLCs compared to primed human PSCs. (B) Heatmap showing expression levels of selected metabolic genes in primed, ICLCs and human ICM (from GSE101571).

FIG. 10. Hematoxylin and eosin staining of teratoma tissues derived from ICLCs shows the structure of all three germ layers: Mesoderm (left panel), Endoderm (middle panel) and Ectoderm (right panel).

FIG. 11. (A) Bar plot showing the expression levels of primed, ICM and TSC markers in TSCLCs differentiated from H9 ICLCs compared to H9 ICLCs. (B) Immunofluorescence microscope images showing expression of TSC markers: GATA3, TFAP2C and KRT7. (C) Principal component analysis comparing the transcriptomes of 4CL converted H9 (H9-4CL), TSCLCs (H9-TSCLC), trophoblast cancer cell line JEG3 and BeWo, and trimester human placenta isolated trophoblasts (EGFR and HLAG). (D) Methylation plot showing CpG methylation status of ELF5 promoter in primed, ICLCs and TSCLCs.

FIG. 12. (A) Table showing the numbers of blastocyst injections using primed, 4CL or e4CL medium converted cells and the number of embryos with labeled cells integrated to ICM and/or TE. (B) Microscope images showing phase contrast (left) and red fluorescence channel (right) of mouse blastocysts injected with DsRed labeled primed human PSCs or ICLCs. (C) Immunofluorescence of injected and uninjected embryos stained with anti-OCT4, anti-CDX2 or counterstained with DAPI.

FIG. 13. (A) Images showing phase contrast (upper) and red fluorescence channel (lower) of E10.5 mouse embryo (left), placenta (middle) and yolk sac (right). (B) Immunofluorescence images showing expression of GATA6 (red) and human nuclei antigen (hN) (green) in E10.5 mouse embryos. Nuclei were counterstained with DAPI (blue). (C) Immunofluorescence images showing expression of DsRed (red) and GATA3 (green) in E10.5 mouse placenta. Nuclei were counterstained with DAPI (blue) in a placental tissue section.

FIG. 14. (A) Microscope images showing phase contrast of self-forming blastoids from ICLCs. (B) Immunofluorescence images of self-forming blastoids stained with anti-OCT4 (red), anti-GATA3 (green) antibodies, or nuclear counterstain DAPI (blue).

FIG. 15. Bar chart showing expression levels of ICM and primed markers in H9, H1, HUES1 and WIBR3 human ESC lines which had been converted to ICLC using 4CL medium 1.

FIG. 16. A bar chart of RT-qPCR data showing that a panel of preimplantation ICM markers KLF17, DNMT3L, DPPA5, STELLA, TFCP2L1, KLF4, MAEL, and REX1 are significantly induced in ICLCs converted on Geltrex™ coated plates using 4CL medium 1.

FIG. 17. A bar chart of RT-qPCR data showing that a panel of preimplantation epiblast markers KLF17, DNMT3L, DPPA5, STELLA, TFCP2L1, KLF4, MAEL, and REX1 are significantly induced in ICLCs converted in suspension using 4CL medium 1. In the bar chart, the left column for each gene represents culture on a feeding cell and the right column represents culture in suspension.

FIG. 18. (A-C) Bar charts of RT-qPCR data showing that a panel of preimplantation ICM markers KLF17, DNMT3L, DPPA5, STELLA, TFCP2L1, KLF4, MAEL, and REX1 are significantly induced in ICLCs converted by 4CL medium 2, 4CL medium 3, and 4CL medium 4, respectively.

FIG. 19. (A) Schematic diagram showing two methods to generate 8CLCs. Briefly, primed human PSCs culture media (e.g. mTeSR1) are changed to either e4CL medium or 4CL media. Cells are then either continually grown in e4CL or switched to e4CL medium after 2 passages in 4CL media. (B) Bar chart showing expression levels of selected naïve pluripotency markers in H9 primed and H9-e4CL cells. (C) Bar chart showing expression levels of selected naïve pluripotency markers in H9-e4CL cells and H9-4CL cells. (D) Induction of 8C specific genes in both methods is similar. (E) Immunofluorescence microscopy images showing expression of ZSCAN4 (green) or DAPI counterstain nuclei (blue) in primed H9, H9-4CL and H9-e4CL.

FIG. 20. (A) 2D scatter plot showing UMAP transformed single-cell RNA-seq data of H9 cells at the primed stage (day 0) and then at day 1, 2, 3 and 5 after being cultured in the e4CL medium. The inventors also included published single-cell RNA-seq gene expression data from human embryo cells of E3, 4, 5, 6 and 7 (from E-MTAB-3929). (B) Heatmap illustrating the expression levels of known 8C markers in primed H9, e4CL converted H9 and human 8C embryo cells (from E-MTAB-3929).

FIG. 21. (A) 2D scatter plot showing UMAP transformed single-cell RNA-seq TE expression of H9 cells at the primed stage (day 0) and then at day 1, 2, 3 and 5 after being cultured in the e4CL medium. The inventors also included published single-cell RNA-seq TE expression data from human embryo cells from day 3, 4, 5, 6 and 7 (from E-MTAB-3929). (B) Heatmap illustrating the RNA gene expression of known naïve TEs in primed H9, e4CL converted H9 and human 8C embryo cells (from E-MTAB-3929).

FIG. 22. Representative images of chromosomes after Giemsa staining to illustrate that H9 (upper left panel), e4CL-H9 (upper right panel), primed UH10 (lower left panel) and e4CL-UH10 (lower right panel) cells had normal karyotype.

FIG. 23. Boxplots showing CpG methylation across genome-wide (left panel) and in 2 kb around TSS (right panel).

FIG. 24. Heatmap showing the CpG methylation at selected ICRs in human ICM (from GSE101571) compared to 8CLCs.

FIG. 25. Differentially accessible chromatin regions in primed human PSCs and cells in the process of conversion to 8CLCs. Regions which are close in primed become open during conversion to 8CLCs (upper panel). Regions which are open in primed turn to close during conversion to 8CLCs (lower panel).

FIG. 26. Heatmap showing expression of selected metabolism genes in primed H9, H9 8CLCs and human 8C embryo cells (from E-MTAB-3929).

FIG. 27. Hematoxylin and eosin staining of teratoma tissue derived from 8CLCs shows the structure of all three germ layers: Mesoderm (left panel), Endoderm (middle panel) and Ectoderm (right panel).

FIG. 28. Bar charts showing that multiple TSC markers such as GATA3, CGA, ELF5, TP63, KRT18, KRT8, PSG6, and CCR7 are significantly induced in TSCLCs compared to undifferentiated 8CLCs.

FIG. 29. (A) Microscope images showing phase contrast (left) or red fluorescence channel (right) of mouse blastocysts injected with DsRed labeled primed human PSCs or 8CLCs. (B) Immunofluorescence images of embryos stained with anti-OCT4, anti-CDX2 or counterstained with DAPI.

FIG. 30. (A) Microscope images showing phase contrast (upper) or red fluorescence channel (lower) of E10.5 mouse embryo (left), placenta (middle) or yolk sac (right). (B) Immunofluorescence images showing expression of GATA6 (red) and hN (green), or counterstained with DAPI (blue) in mouse embryo. (C) Immunofluorescence images showing expression of DsRed (red) and KRT7 (green), or counterstained with DAPI (blue) in mouse placenta.

FIG. 31. (A) Microscope images showing phase contrast of self-forming blastoids from 8CLCs. (B) Immunofluorescence image of self-forming blastoids stained with anti-OCT4 (red) and anti-GATA3 (green) antibodies, or counterstained with DAPI (blue).

FIG. 32. A bar chart of RT-qPCR data showing 8C markers ZSCAN4, ARGFX, TPRX1, ZNF280A, and ZSCAN5B are significantly induced in 8CLCs converted in suspension using e4CL medium. In the bar chart, the left column for each gene represents culture on a feeding cell and the right column represents culture in suspension.

FIG. 33. A bar chart of RT-qPCR data showing 8C markers ZSCAN4, ARGFX, TPRX1, ZNF280A, ZSCAN5B, DUXA, DUXB, and MBD3L2 are significantly induced in 8CLCs converted from multiple hPSC lines. As shown in FIG. 33, the columns for each of these genes in the primed HN10 and UH10 are basically absent, indicating that the expression of these genes in the primed HN10 and UH10 are extremely low.

FIG. 34. A bar chart of RT-qPCR data showing that expression levels of preimplantation ICM markers KLF17, DNMT3L, DPPA5, STELLA, TFCP2L1, KLF4, MAEL, and REX1 in ICLC converted by 4CL medium 1 under normoxia are comparable to that of hypoxia.

FIG. 35. Bar charts of RT-qPCR data showing mouse 2C markers Zscan4, Zscan4b, Zscan4c, Zscan4d, Dux, Tcstv1, Tcstv3, Gm4340, Zfp352, and Dub1 are significantly induced in 2C-like cells converted from multiple mouse ESC lines compared to mouse ESCs cultured in serum/Lif medium and other naïve conversion media known in the art. The five columns for each gene in the upper panel respectively represent, from left to right, E14 serum+Lif, E14 4CL, E14 5iLAF R14 PXGL and E14 e4CL; The five columns for each gene in the lower panel respectively represent, from left to right, Mervl-GFP serum+Lif, Mervl-GFP 4CL, Mervl-GFP 5iLAF, Mervl-GFP PXGL and Mervl-GFP e4CL.

FIG. 36. A bar chart of RT-qPCR data demonstrating that preimplantation ICM markers KLF17, DNMT3L, DPPA5, STELLA, TFCP2L1, KLF4, MAEL, and REX1 are significantly induced in ICLCs converted using 4CL medium supplemented with different dosage of either PD0325901, DZNep, or TSA compared to primed human PSCs cells. The five columns for each gene in the right panel respectively represent, from left to right, DZNep-0 nM, DZNep-5 nM, DZNep-10 nM, DZNep-20 nM and DZNep-50 nM.

FIG. 37. (A) Bar charts of RT-qPCR data showing the knockdown efficiency of snRNA against TPRX1, KHDC1L, and TRIM60. (B) Bar charts of RT-qPCR data demonstrating that 8C specific gene induction is prohibited by TPRX1, KHDC1L, and TRIM60 knockdown during ICLC to 8CLC conversion.

### DETAILED DESCRIPTION OF THE INVENTION

Current methods for derivation and maintenance of naïve human PSCs (Chan, Goke et al. 2013; Takashima, Guo et al. 2014; Theunissen, Powell et al. 2014) that exhibit some characteristics of mouse ESCs, which are close to mouse preimplantation ICM. The derivation of naïve human PSCs using current methods is problematic, such as being lengthy, producing variable levels of naïve specific genes, transgene dependency for naïve induction, genomic instability and imprinting loss, inability of multi-lineage differentiation and inefficient or no proper chimera formation competency. None of these studies reported generation of cells that are close to 8C stage.

To overcome the problems mentioned above, the inventors first conducted a screen with a panel of inhibitors that target epigenetic regulators and different signaling pathways relevant to human preimplantation ICM development, and found that three basic modulators (JAK/STAT3 activator, MAPK/ERK inhibitor and tankyrase inhibitor) could activate a molecular network governing preimplantation ICM-like state in primate PSCs. The inventors also found that SAH/PRC/EZH2 inhibitor and HDAC inhibitor permit rewiring the epigenetic landscape of the cultured PSCs to be more similar to human preimplantation ICM, which transforms conventional primate PSCs to ICLCs that possess all major features of human preimplantation ICM as described in the Background section.

Thus, multiple methods and chemically defined culture mediums that facilitate robust derivation of primate ICLCs are provided in the subject application. The methods described herein can be applied to a number of human and non-human primate PSC lines, which are either at a primed state as validated by the presence of pluripotency surface markers such as SSEA-3, SSEA-4, TRA-1-81, and TRA-1-60, or at a preimplantation ICM-like state as validated by expression of genes such as DNMT3L, STELLA, DPPA5, and KLF17. The primate PSC lines that can be used in the present application include but are not limited to conventional primate PSCs and ICM-like PSCs. The methods described herein can also be applied to the isolation of ICLCs from primate preimplantation ICM. The described methods are transgenic free and straight forward as provided primate PSCs can be converted to ICLCs in one culture condition in approximately 2 weeks.

To the best of our knowledge, so far there is no proper method for inducing primate 8CLCs in vitro. To achieve this, the inventors further optimized the recipe for inducing ICLCs, and found that by increasing only the dosage of SAH/PRC/EZH2 inhibitor and HDAC inhibitor in the medium, primed human PSCs and/or ICLCs could be converted to 8CLCs. Thus, a chemically defined culture medium that facilitates derivation of primate 8CLCs is provided in the subject application. The method described herein can be applied to a number of human and non-human primate PSC lines, which are either at a primed state as validated by the presence of pluripotency surface markers such as SSEA-3, SSEA-4, TRA-1-81, and TRA-1-60, or at a preimplantation ICM-like state as validated by expression of genes such as DNMT3L, STELLA, DPPA5, and KLF17. The primate PSC lines that can be used in the present application include but are not limited to primed primate PSCs and ICM-like PSCs. The methods described herein can also be applied to the isolation of 8CLCs from primate 8C embryos. The described method is transgenic free and straight forward as the provided primate PSCs can be converted to 8CLCs in one culture condition in approximately 1 week.

Detailed descriptions of the invention will be described below. It should be understood that features described in various embodiments could be combined with each other to form preferred technical solutions, which are also contemplated in the scope of the subject application.

### I. Terms

Unless otherwise specified, all terms used herein have the meanings generally understood by those skilled in the art. In order to facilitate the understanding of the invention, some terms used herein are defined as follows.

The singular “one” and “this” used in the description and claims include plural references, unless the context clearly states otherwise. For example, the term “(one) cell” includes a plurality of cells, including a mixture thereof.

All digital indicators, such as pH, temperature, time, concentration and molecular weight, including range, are approximate. It is important to understand that, although not always explicitly stated, all digital indicators are preceded by the term “about”. It is also to be understood that, although not always explicitly described, the reagents described herein are only examples and their equivalents are known in the art.

The term “basal medium” used herein refers to any medium capable of supporting cell growth. Basal media provide standard inorganic salts such as zinc, iron, magnesium, calcium, and potassium, as well as vitamins, glucose, buffer systems, and key amino acids. The basal medium which can be used for in the subject application includes but is not limited to Dulbecco's modified eagle's medium (DMEM), minimal essential medium (MEM), basal medium Eagle (BME), RPMI1640, F10, F12, a minimal essential medium (α MEM), Glasgow's minimal essential medium (GMEM), Iscove's modified Dulbecco's medium, Neurobasal Medium, and DMEM/F12. Those skilled in the art know how to select a basal culture medium suitable for the cultured cells. In a preferred embodiment, the basal medium used in the subject application is a mixture of DMEM/F12 and Neurobasal Medium in a ratio of 1:1 (w/w).

The term “serum-free” means the absence of any blood serum of any species including, but not limited to, the absence of fetal bovine serum, calf bovine serum, human serum, or the like, or combinations thereof.

The term “serum replacement” as used herein refers to additives used in a basal culture medium to partially or completely replace serum to support cell survival and growth. A serum replacement generally includes factors such as insulin, metalloprotein, microelement, vitamin and the like. These factors are generally not contained in the basal culture medium, but are provided by a serum commonly used to culture cells. Serum replacement include at least one or more of the following components that support cell growth: one or more insulin and insulin substitutes, one or more metalloprotein and metalloprotein substitutes, one or more trace elements, one or more vitamins, one or more amino acids, one or more multiple hormones and hormone-like compounds, serum albumin or serum albumin substitutes, and one or more lipids, etc. A variety of commercial serum replacement are known in the art, including KOSR, N2, B27, Insulin-Transferrin-Selenium Supplement (ITS), G5, etc., which are easily obtained by those skilled in the art. These replacements each have a defined composition, so the concentration of each component can be determined according to their respective proportions in the culture medium.

Those skilled in the art can easily configure serum replacement according to the prior art, the type of cells to be cultured and other aspects. Preferably, the serum replacement used herein is a mixed additive obtained by mixing KOSR, N2 and/or B27 in a certain proportion. More preferably, the serum replacement used herein is a mixture of N2 and B27 in a ratio of 1:1 (w/w).

The term “primate” or “primate animal” used herein refers to animals belonging to Primates, including human and non-human primates. The non-human primates include animals of Prosimian and Simiae. Specific non-human primates include but are not limited to Macaques, lemurs, gibbons, orangutans, and baboons.

The term “Pluripotent Stem cells” (PSCs) used herein refers to pluripotent cells derived from embryo at any time before gastrulation and iPSCs generated from somatic cell reprogramming. Depending on their source and method of culture, the PSCs may be at alternative states, in which including primed PSCs, naïve PSCs, extended PSCs and expanded potential stem cells (Gafni et al., 2013; Gao et al., 2019; Takashima et al., 2014; Theunissen et al., 2014; Yang et al., 2017). PSCs have the characteristic of being capable under appropriate conditions of producing progeny of different cell types that are derivatives of all of the three germinal layers (endoderm, mesoderm, and ectoderm), according to a standard art-accepted test, such as the ability to form a teratoma in 8-12 week old SCID mice, and may also being capable under appropriate conditions of producing different cell types of placenta. PSC cultures are described as “undifferentiated” when a substantial proportion of stem cells and their derivatives in the population display morphological characteristics of undifferentiated cells, distinguishing them from differentiated cells of embryo or adult origin. It is understood that colonies of undifferentiated cells within the population may be surrounded by neighboring cells that are differentiated.

The subject application can be practiced using stem cells of various types. Particularly suitable for use in the subject application are primate PSCs. Non-limiting examples are primary cultures or established lines of ESCs and iPSCs. PSCs of any non-primate mammals can also be used to practice the subject application.

In one or more embodiments, the primate PSCs that may be used in the present application can be selected from a group consisting of:
(i) cells from an ESC line and/or an ECC line;
(ii) cells from an iPSC line;
(iii) cells from ICM of a preimplantation blastocyst cultured in vitro;
(iv) cells from ICM of a post-implantation blastocyst cultured in vitro; and
(v) cells from an embryo of 8C stage to morula stage cultured in vitro.

Non-limiting PSCs include but are not limited to any established cell lines in the art, such as human ESC lines, such as H1 (male), H9 (female), HN10 (female), HUES1 (female) and WIBR3 (female); human iPSC lines, such as CBC14 (female), C11 (female), Phoenix (female), DiPS 1016SevA (male), STiPS O-XX1 (female), and UH10 (male).

### II. Culture Media

The culture media disclosed herein are chemically defined media, which can efficiently convert primate PSCs from a primed state to a preimplantation ICM-like state to produce ICLCs within 2 weeks without picking colonies. The culture media of the subject application can also convert primate PSCs from a primed state and/or a preimplantation ICM-like state to an 8C like state to produce 8CLCs in approximately one week. Therefore, this kind of culture media can also be called as “conversion culture media” in the subject application. In some embodiments, the culture medium of the present application can also support derivation, survival after passage and/or revival, self-renewal, and proliferation of cells in a preimplantation ICM-like state. In some other embodiments, the culture medium of the present application can also support survival after passage and/or revival, self-renewal and proliferation of cells in a preimplantation ICM-like state on an extracellular matrix without the need for feeder cells or conditioned medium. In some embodiments, the culture medium of the present application can support survival after passage and/or revival, self-renewal and proliferation of cells in a preimplantation ICM-like state in suspension without the need for feeder cells or conditioned medium. In some other embodiments, the culture medium of the present application can support survival after passage and/or revival, self-renewal, and proliferation of cells in a preimplantation ICM-like state on feeder cells. Preferably, the chemically defined culture media of the subject application are serum-free.

The culture media of the subject application contain a basal medium capable of supporting cell growth, especially capable of supporting growth of human and non-human primate PSCs, supplemented with a PRC inhibitor and/or an EZH2 inhibitor and a HDAC inhibitor, and optionally one or more components selected from a group consisting of L-ascorbic acid, an activator of JAK/STAT3 signaling, an inhibitor of MAPK/ERK signaling and a tankyrase inhibitor. Preferred basal medium used in the subject application is a mixture of Advanced DMEM/F12 and Neurobasal Medium in a ratio of 1:1 (v/v). It should be understood that the inhibitor of SAH can also inhibit PRC and EZH2. Therefore, in some embodiments, the PRC inhibitor and/or EZH2 inhibitor (PRC/EZH2 inhibitor) is a SAH inhibitor. In the context of the subject application, the term “SAH/PRC/EZH2 inhibitor” refers to an inhibitor of SAH, PRC and/or EZH2.

The presence of SAH/PRC/EZH2 inhibitor in the described culture conditions is critical for inducing multiple regulators including STELLA, DNMT3L, and MAEL that govern the human naïve pluripotency network. STELLA is a DNA methylation regulator. Its ectopic over-expression in somatic cells can induce comprehensive DNA demethylation by interfering with the function of UHRF1, a DNA methylation regulator. The dysfunction of UHRF1 caused by STELLA deletion would lead to the accumulation of abnormal DNA methylation during oogenesis (Li et al., 2018). The induction of STELLA was found to be dose dependent. The inventors further uncovered the functional role of STELLA and found that STELLA knock out hinders the induction of ICLCs and 8CLCs. During primed PSCs to ICLCs conversion, preimplantation ICM markers including KLF17, DPPA5, DNMT3L, TFCP2L1, and MAEL fail to be induced upon STELLA deletion. During primed PSCs and ICLCs to 8CLCs conversion, 8C markers including TPRX1, TRIM60, KHDC1L, YPEL2, ALPG, ZNF280F, FAM151A, and CCNA1 fail to be induced upon STELLA deletion. As demonstrated in the subject application, global DNA methylation levels are significantly higher in STELLA knock out cells compared to wild-type cells during 4CL or e4CL conversion. Thus, STELLA is necessary for controlled DNA demethylation during conversion to ICLCs and 8CLCs. Altogether, the subject application discovers that adding SAH/PRC/EZH2 inhibitors promotes induction of human ICLCs and 8CLCs through rewiring histone modification and DNA methylation landscape.

Any substances that can act as an inhibitor of SAH/PRC/EZH2 can be used in the culture media of the subject application, which include but are not limited to DZNep (CAS NO: 102052-95-9, a SAH inhibitor) and CPI-1205 (CAS NO: 1621862-70-1, a PRC/EZH2 inhibitor). The SAH/PRC/EZH2 inhibitors can be used alone or in combination in the culture media of the subject application, generally in their respective conventional amounts which will not lead to cell death. For example, DZNep can be used in the media at a final concentration of 5 to 80 nM, preferably 5 to 50 nM, and CPI-1205 can be used in the media at a final concentration of 0.5 to 5 mM, preferably 1 to 3 mM. In one or more embodiments, the SAH/PRC/EZH2 inhibitor is a PRC inhibitor.

Any substances that can act as an inhibitor of HDAC can be used in the culture media of the subject application, which include but are not limited to TSA, VPA and NaB. The HDAC inhibitors can be used alone or in combination in the culture media of the subject application, generally in their respective conventional amounts which will not lead to cell death. For example, TSA can be used in the media at a final concentration of 3 to 30 nM, preferably 3 to 25 nM, VPA can be used in the media at a final concentration of 0.25 to 2 mM, preferably 0.5 to 1.5 mM, and NaB can be used in the media at a final concentration of 0.25 to 2 mM, preferably 0.5 to 1.5 mM.

The inventors also find that 8CLCs can be obtained with the culture media of the subject application from primed PSCs and/or ICLCs when both the SAH/PRC/EZH2 inhibitor and the HDAC inhibitor are used in a higher concentration. Specifically, in some embodiments, in order to produce 8CLCs, DZNep may be used at a concentration of 40 nM or higher, such as 40 to 80 nM, preferably about 50 nM; CPI-1205 may be used at a concentration of 2 mM or higher, such as 2 to 5 mM, preferably about 3 mM; TSA may be used at a concentration of 10 nM or higher, such as 10 to 30 nM, preferably about 20 nM; VPA may be used at a concentration of 1.0 mM or higher, such as 1.0 to 2.0 mM, preferably about 1.5 mM; and NaB may be used at a concentration of 1.0 mM or higher, such as 1.0 to 2.0 mM, preferably about 1.5 mM, when each of them is used alone. It should be understood when two or more of the SAH/PRC/EZH2 inhibitors or two or more of the HDAC inhibitors are used, the final concentration of each SAH/PRC/EZH2 inhibitor or each HDAC inhibitor should be reduced to an amount sufficient to induce 8CLCs by combination of these SAH/PRC/EZH2 inhibitors or HDAC inhibitors. These amounts could readily be determined by the skilled artisan based on the disclosure of the subject application and the conventional knowledge of the art.

Furthermore, it should also be understood that excessive amount of SAH/PRC/EZH2 inhibitor and HDAC inhibitor may cause cell death. Thus, in order to induce ICLCs while reducing cell death as much as possible, either one of or both the SAH/PRC/EZH2 inhibitor and HDAC inhibitor may be used in a relatively low concentration. Specifically, DZNep can be used at a final concentration of 5 to 15 nM, preferably about 10 nM, CPI-1205 can be used at a final concentration of 0.5 to 3 mM, preferably about 1 mM, TSA can be used at a final concentration of 3 to 10 nM, preferably 4 to 6 nM, more preferably about 5 nM, VPA can be used at a final concentration of 0.25 to 1 mM, preferably 0.5 mM, and NaB can be used at a final concentration of 0.25 to 1 mM, preferably 0.5 mM, when each of them is used alone. In some embodiments, the SAH/PRC/EZH2 inhibitor can be used in a relatively high concentration, for example, DZNep can be used at a final concentration of 5 to 80 nM, preferably 5 to 50 nM, CPI-1205 can be used in the media at a final concentration of 0.5 to 5 mM, preferably 1 to 3 mM, while the HDAC inhibitor is used in a relatively low concentration, for example, TSA is used at a final concentration of 3 to 10 nM, preferably 4 to 6 nM, VPA is used at a final concentration of 0.25 to 0.5 mM, and NaB is used at a final concentration of 0.25 to 0.5 mM. In some embodiments, the SAH/PRC/EZH2 inhibitor is used in a relatively low concentration, for example, DZNep is used at a final concentration of 5 to 15 nM, CPI-1205 is used at a final concentration of 0.5 to 2 mM, while the HDAC inhibitor can be used in a relatively high concentration, for example, TSA can be used in the media at a final concentration of 3 to 30 nM, preferably 3 to 25 nM, VPA can be used in the media at a final concentration of 0.25 to 2 mM, and NaB can be used in the media at a final concentration of 0.25 to 2 mM. Such culture media can convert primate PSCs to ICLCs.

L-ascorbic acid is found to improve generation and maintenance of mouse iPSCs (close to mouse ESCs) from somatic cells through enhancing Jumonji-domain-containing histone demethylase, as described in Application number CN 200910041331.9, the content of which is incorporated herein by reference. Therefore, the inventors hypothesize that L-ascorbic acid has similar effects on formation of primate preimplantation ICM-like state. With proper testing, the inventors found that it potently increases expression levels of ICM specific genes such as DNMT3L, STELLA, DPPA5, and KLF17, when used at a final concentration of 40 to 70 ng/mL. In a preferred embodiment, L-ascorbic acid is used at a final concentration of about 50 ng/mL.

Derivatives of L-ascorbic acid can also be used in the subject application, which refer to similar compounds with similar structure and antioxidant activity to L-ascorbic acid. The derivatives are more stable or easy to be absorbed by cells while maintaining the biological activity of L-ascorbic acid. The derivatives of L-ascorbic acid include but are not limited to L-ascorbic acid phosphate and L-ascorbic acid organic ester, such as L-ascorbic acid palmitate. Amount of the derivative in the subject media is not limited, but it generally should be sufficient to produce sufficient amount of L-ascorbic acid as defined above.

One or more activators of JAK/STAT3 signaling can be added into the culture media that can cooperate to induce subset of early embryo specific gene of the subject application. Any known JAK/STAT3 activators can be used, especially those generally used in culture of stem cells, are preferred. Exemplary final concentration of the JAK/STAT3 activators may be in a range of 10 to 50 ng/mL. One kind of such JAK/STAT3 activators is LIF. LIF used herein refers to leukemia inhibitory factor, which is a growth factor commonly added to culture stem cells. Preferably LIF is a human LIF. JAK/STAT3 activator can be used in an amount commonly used in culturing stem cells. For example, for LIF, especially human LIF, its final concentration in the culture media of the subject application may be in a range of from 10 to 50 ng/mL, preferably 10 to 30 ng/mL, more preferably about 20 ng/mL.

One or more inhibitors of MAPK/ERK signaling can be added into the culture media which help to reduce DNA methylation in cooperation with other components in the media of the subject application. Any known MAPK/ERK inhibitors can be used, especially those generally used in culture of stem cells, are preferred. One kind of such MAPK/ERK inhibitors is PD0325901 (CAS No.: 391210-10-9). MAPK/ERK inhibitors can be used in an amount commonly used in culturing stem cells. Exemplary final concentration of the MAPK/ERK inhibitors may be in a range of 0.5 to 3 nM, preferably 0.5 to 1.5 μM. For example, for PD0325901, its final concentration in the culture media of the subject application may be in a range of 0.5 to 3 nM, preferably 0.5 to 1.5 nM, more preferably about 1 μM.

One or more tankyrase inhibitors, which inhibit canonical WNT signaling, can be added into the culture media of the subject application. Any known tankyrase inhibitors can be used, especially those generally used in culture of stem cells, are preferred, which include but are not limited to IWR1 (CAS No.: 1127442-82-3) and XAV939 (CAS No.: 284028-89-3). Tankyrase inhibitors can be used in an amount commonly used in culturing stem cells. Exemplary final concentration of the tankyrase inhibitors may be in a range of from 2 to 8 nM, preferably 3 to 6 μM. For example, for IWR1 and XAV939, their respective final concentration in the culture media of the subject application may be in a range of from 2 to 8 nM, preferably 3 to 6 nM, more preferably about 5 μM. Two or more tankyrase inhibitors can be used in combination, with reduced amount for each of the inhibitors.

In one or more preferred embodiments, the culture medium of the present application comprises DZNep at a final concentration of 5 to 15 nM or CPI-1205 at a final concentration of to 2 mM; TSA at a final concentration of from 3 to 30 nM, or VPA at a final concentration of to 2 mM or NaB at a final concentration of 0.25 to 2 mM, preferably, TSA at a final concentration of from 3 to 10 nM, or VPA at a final concentration of 0.25 to 1 mM or NaB at a final concentration of 0.25 to 1 mM; L-ascorbic acid at a final concentration of 40 to 70 ng/mL; LIF at a final concentration of 10 to 30 ng/mL; PD0325901 at a final concentration of 0.5 to 1.5 μM; and IWR1 or XAV939 each at a final concentration of 3 to 6 μM. In one or more embodiments, the culture medium of the present application comprises DZNep at a final concentration of 5 to 80 nM, preferably 5 to 50 nM or CPI-1205 at a final concentration of 0.5 to mM, preferably 0.5 to 3 mM; TSA at a final concentration of from 3 to 10 nM, or VPA at a final concentration of 0.25 to 0.5 mM or NaB at a final concentration of 0.25 to 0.5 mM; L-ascorbic acid at a final concentration of 40 to 70 ng/mL; LIF at a final concentration of 10 to 30 ng/mL; PD0325901 at a final concentration of 0.5 to 1.5 μM; and IWR1 or XAV939 each at a final concentration of 3 to 6 μM. More preferably, the culture medium of the present application comprises 10 nM DZNep or 1 mM CPI-1205; 5 nM TSA, or 0.5 mM VPA, or 0.5 mM NaB; 50 ng/mL L-ascorbic acid; 20 ng/mL LIF; 1 μM PD0325901; and 5 μM IWR1 or 5 μM XAV939. These culture media are preferably used to convert primate PSCs to ICLCs.

In one or more preferred embodiments, the culture medium of the present application comprises DZNep at a final concentration of 40 to 70 nM or CPI-1205 at a final concentration of 2 to 4 mM; TSA at a final concentration of from 10 to 30 nM, or VPA at a final concentration of to 1.5 mM or NaB at a final concentration of 0.5 to 1.5 mM; L-ascorbic acid at a final concentration of 40 to 70 μg/mL; LIF at a final concentration of 10 to 30 ng/mL; PD0325901 at a final concentration of 0.5 to 1.5 μM; and IWR1 or XAV939 each at a final concentration of 3 to 6 μM. More preferably, the culture medium of the present application comprises 50 nM DZNep or 3 mM CPI-1205; 20 nM TSA, or 1 mM VPA, or 1 mM NaB; 50 μg/mL L-ascorbic acid; 20 ng/mL LIF; 1 μM PD0325901; and 5 μM IWR1 or 5 μM XAV939. These culture media are preferably used to convert primate PSCs or ICLCs to 8CLCs.

The culture media of the subject application can further comprise at least one or more additives selected from a group consisting of an extracellular matrix, an activator of ACTIVIN/NODAL signaling and a ROCK inhibitor.

Compared to primed human PSCs, expression levels of NODAL (an activator of ACTIVIN/NODAL signaling) are increased in ICLCs and 8CLCs derived by methods described herein. This observation indicates that ACTIVIN/NODAL signaling is endogenously/automatically activated in the conversion process and during self-renewal. Therefore, in some embodiments of the subject application, the culture medium further comprises an activator of ACTIVIN/NODAL signaling to accelerate the conversion process. Any known activators of ACTIVIN/NODAL signaling can be added to the culture medium of the subject application, which include but are not limited to human ACTIVIN A and human NODAL, the amino acid sequences of which are well known in the art. Human ACTIVIN A or human NODAL can be present in the culture medium of the present application at a final concentration of 10 to 25 ng/mL, preferably about 20 ng/mL. A combination of human ACTIVIN A and human NODAL can also be used. Generally, the total concentration of human ACTIVIN A and human NODAL in the culture medium is in a range of 10 to 25 ng/mL, preferably about 20 ng/mL.

Upon conversion to ICLCs and/or 8CLCs, inhibition of ROCK signaling is no longer required for survival after passaging as single cells. Nevertheless, supplying with a ROCK inhibitor at a low concentration increases the yield of ICLCs and 8CLCs, which will be beneficial for scaling up the culture. Thus, in some embodiments of the invention, the culture medium further includes a ROCK inhibitor. Any known ROCK inhibitors can be used in the culture medium of the present application, which include but are not limited to Y27632 (CAS No.: 146986-50-7), thiazovivin (CAS No.: 1226056-71-8) and hydroxyfasudil (CAS No.: 105628-72-6). ROCK inhibitor can be used at a final concentration in a range of 0.5 to 2 μM, preferably about 1 μM. Two or more ROCK inhibitors can be used in combination, with their total concentration in the culture medium in a range of 0.5 to 2 μM, preferably about 1 μM.

The inventors find that when the PSCs are cultured in the culture medium of the subject application, they can be converted and maintained in a suspension culture without feeder cells, and the converted cells can self-renew and propagate as sphere-like colonies. Therefore, in some embodiments of the present application, the described methods, culture conditions and culture media are feeder-free.

In some other embodiments, the inventors find that during conversion and maintenance, supplying an extracellular matrix will promote sphere-shape colonization. In this condition, 90% or more of PSCs could be transformed into dome-shape colonies during conversion, which express ICM marker such as DNMT3L and KLF17. Therefore, in some embodiments, an extracellular matrix is used in the medium for culturing the ICLCs and 8CLCs. Extracellular matrix is a solubilized basement membrane preparation extracted from the Engelbreth-Holm-Swarm mouse sarcoma (Matrigel™ or Geltrex™ or ECM™) or a matrix that includes human matrix proteins collagen IV and at least one member selected from fibronectin, laminin, and vitronectin. The extracellular matrix generally is present in an amount of 0.1% to 0.5% (v/v) in the culture medium of the present application. A combination of different kinds of extracellular matrices can be used, if necessary, and the total amount thereof should also be in the range of 0.1% to 0.5% (v/v) in the culture medium. Preferably, the extracellular matrix generally is present in an amount of about 0.2% (v/v) in the culture medium of the present application.

Therefore, in one or more preferred embodiments, the culture medium of the present application comprises:
(A) DZNep at a final concentration of 5 to 15 nM or CPI-1205 at a final concentration of to 2 mM, and TSA at a final concentration of from 3 to 30 nM, or VPA at a final concentration of 0.25 to 3 mM or NaB at a final concentration of 0.25 to 3 mM, preferably TSA at a final concentration of from 3 to 10 nM, or VPA at a final concentration of 0.25 to 1 mM or NaB at a final concentration of 0.25 to 1 mM; or DZNep at a final concentration of 5 to 80 nM, preferably 5 to 50 nM or CPI-1205 at a final concentration of 0.5 to 5 mM, preferably 0.5 to 3 mM, and TSA at a final concentration of from 3 to 10 nM, or VPA at a final concentration of to 0.5 mM or NaB at a final concentration of 0.25 to 0.5 mM;
(B) L-ascorbic acid at a final concentration of 40 to 70 μg/mL;
(C) LIF at a final concentration of 10 to 30 ng/mL;
(D) PD0325901 at a final concentration of 0.5 to 1.5 μM;
(E) IWR1 or XAV939 at a final concentration of 3 to 6 μM; and is further supplemented with:
(1) ACTIVIN A or NODAL at a final concentration of 10 to 25 ng/mL; Y27632, thiazovivin or hydroxyfasudil at a final concentration in a range 0.5 to 2 μM; and an extracellular matrix in an amount of 0.1% to 0.5% (v/v); or
(2) ACTIVIN A or NODAL at a final concentration of 10 to 25 ng/mL; and Y27632, thiazovivin or hydroxyfasudil at a final concentration in a range 0.5 to 2 μM; or
(3) ACTIVIN A or NODAL at a final concentration of 10 to 25 ng/mL; and an extracellular matrix in an amount of 0.1% to 0.5% (v/v); or
(4) Y27632, thiazovivin or hydroxyfasudil at a final concentration in a range 0.5 to 2 μM; and an extracellular matrix in an amount of 0.1% to 0.5% (v/v); or
(5) ACTIVIN A or NODAL at a final concentration of 10 to 25 ng/mL; or Y27632, thiazovivin or hydroxyfasudil at a final concentration in a range 0.5 to 2 μM; or an extracellular matrix in an amount of 0.1% to 0.5% (v/v). These culture media are preferably used to convert primate PSCs to ICLCs.

More preferably, the culture medium of the present application comprises 10 nM of DZNep or 1 mM of CPI-1205; 5 nM of TSA, or 0.5 mM of VPA, or 0.5 mM of NaB; 50 ng/mL of L-ascorbic acid; 20 ng/mL of LIF; 1 μM of PD0325901; and 5 μM of IWR1 or 5 μM of XAV939; and is further supplemented with (1) 20 ng/mL of ACTIVIN A or NODAL, 1 μM of Y27632, thiazovivin or hydroxyfasudil, and 0.2% (v/v) of an extracellular matrix; or (2) 20 ng/mL of ACTIVIN A or NODAL, and 1 μM of Y27632, thiazovivin or hydroxyfasudil; (3) 20 ng/mL of ACTIVIN A or NODAL, and 0.2% (v/v) of an extracellular matrix; or (4) 1 μM of Y27632, thiazovivin or hydroxyfasudil, and 0.2% (v/v) of an extracellular matrix; or (5) 20 ng/mL of ACTIVIN A or NODAL, or 1 μM of Y27632, thiazovivin or hydroxyfasudil, or 0.2% (v/v) of an extracellular matrix. These culture media are preferably used to convert primate PSCs to ICLCs.

In one or more preferred embodiments, the culture medium of the present application comprises DZNep at a final concentration of 40 to 70 nM or CPI-1205 at a final concentration of 2 to 4 mM; TSA at a final concentration of from 10 to 30 nM, or VPA at a final concentration of to 1.5 mM or NaB at a final concentration of 0.5 to 1.5 mM; L-ascorbic acid at a final concentration of 40 to 70 ng/mL; LIF at a final concentration of 10 to 30 ng/mL; PD0325901 at a final concentration of 0.5 to 1.5 μM; and IWR1 or XAV939 each at a final concentration of 3 to 6 μM; and is further supplemented with:
(1) ACTIVIN A or NODAL at a final concentration of 10 to 25 ng/mL; Y27632, thiazovivin or hydroxyfasudil at a final concentration in a range 0.5 to 2 μM; and an extracellular matrix in an amount of 0.1% to 0.5% (v/v); or
(2) ACTIVIN A or NODAL at a final concentration of 10 to 25 ng/mL; and Y27632, thiazovivin or hydroxyfasudil at a final concentration in a range 0.5 to 2 μM; or
(3) ACTIVIN A or NODAL at a final concentration of 10 to 25 ng/mL; and an extracellular matrix in an amount of 0.1% to 0.5% (v/v); or
(4) Y27632, thiazovivin or hydroxyfasudil at a final concentration in a range 0.5 to 2 μM; and an extracellular matrix in an amount of 0.1% to 0.5% (v/v); or
(5) ACTIVIN A or NODAL at a final concentration of 10 to 25 ng/mL; or Y27632, thiazovivin or hydroxyfasudil at a final concentration in a range 0.5 to 2 μM; or an extracellular matrix in an amount of 0.1% to 0.5% (v/v). These culture media are preferably used to convert primate PSCs or ICLCs to 8CLCs.

More preferably, the culture medium of the present application comprises 50 nM DZNep or 3 mM CPI-1205; 20 nM TSA, or 1 mM VPA, or 1 mM NaB; 50 ng/mL L-ascorbic acid; 20 ng/mL LIF; 1 μM PD0325901; and 5 μM IWR1 or 5 μM XAV939; and is further supplemented with (1) 20 ng/mL of ACTIVIN A or NODAL, 1 μM of Y27632, thiazovivin or Hydroxyfasudil, and 0.2% (v/v) of an extracellular matrix; or (2) 20 ng/mL of ACTIVIN A or NODAL, and 1 μM of Y27632, thiazovivin or hydroxyfasudil; (3) 20 ng/mL of ACTIVIN A or NODAL, and 0.2% (v/v) of an extracellular matrix; or (4) 1 μM of Y27632, thiazovivin or hydroxyfasudil, and 0.2% (v/v) of an extracellular matrix; or (5) 20 ng/mL of ACTIVIN A or NODAL, or 1 μM of Y27632, thiazovivin or hydroxyfasudil, or 0.2% (v/v) of an extracellular matrix. These culture media are preferably used to convert primate PSCs or ICLCs to 8CLCs.

In addition to the above-mentioned components, other additives commonly used in a culture medium for culturing stem cells can also be added in the culture medium of the subject application, which include but are not limited to serum replacement, such as N2 and/or B27; alternative carbon source, such as pyruvate, such as sodium pyruvate; non-essential amino acid; L-glutamine or its alternative, such as Glutamax™ supplement comprising L-alanyl-L-glutamine dipeptide in 0.85% NaCl; and antibiotic, such as penicillin, streptomycin, or a mixture of penicillin and streptomycin. These additives can be used in an amount commonly used in cell culture, especially culture of stem cells.

### III. Kits and Compositions

Disclosed also include kits containing the culture medium of the present application, or all or a portion of components of the culture medium of the present application for formulating the culture medium.

In some embodiments, kits of the present application contain a ready-for-use culture medium, the components of which are described in any of the above-mentioned embodiments of the culture medium. In some embodiments, kits of the present application contain a conversion culture medium for converting primate PSCs to ICLCs as described in any embodiments of the present application and/or a conversion culture medium for converting primate PSCs or ICLCs to 8CLCs as described in any embodiments of the present application.

In other embodiments, kits of the present application contain at least a SAH/PRC/EZH2 inhibitor and a HDAC inhibitor, which may be packaged individually or may be provided as a mixture in one container. Kits may further contain one or more components selected from a group consisting of L-ascorbic acid, an activator of JAK/STAT3 signaling, an inhibitor of MAPK/ERK signaling, and a tankyrase inhibitor, which, when present, may be individually packaged or provided in a mixture of any combination of components. Preferably, kits may contain a SAH/PRC/EZH2 inhibitor and a HDAC inhibitor, and L-ascorbic acid, an activator of JAK/STAT3 signaling, an inhibitor of MAPK/ERK signaling, and a tankyrase inhibitor. Furthermore, kits may also contain one or more components selected from a group consisting of an activator of ACTIVIN/NODAL signaling and a ROCK inhibitor. Conventional extracellular matrix, such as Matrigel™, Geltrex™ and ECM™, may be contained in the kits. Preferably, kits further contain basal culture medium, such as one or more of the basal culture media described herein, e.g. DMEM/F12 (1:1) and/or Neurobasal Medium, and other components known to be used for culture of stem cell, such as serum replacement, such as N2 and/or B27, alternative carbon source, such as pyruvate, such as sodium pyruvate, non-essential amino acid, L-glutamine or its alternative, such as Glutamax™ supplement comprising L-alanyl-L-glutamine dipeptide in 0.85% NaCl, and antibiotic. Amounts of all these components should be sufficient to formulate the culture medium of the subject application.

Instructions may be contained in the kits, which may comprise text about the formulation of the culture medium and use thereof.

In some embodiments, compositions comprising a SAH/PRC/EZH2 inhibitor and a HDAC inhibitor are also provided. The compositions may further comprise one or more components selected from a group consisting of L-ascorbic acid, an activator of JAK/STAT3 signaling, an inhibitor of MAPK/ERK signaling, and a tankyrase inhibitor. Furthermore, the compositions may also contain one or more components selected from a group consisting of an activator of ACTIVIN/NODAL signaling and a ROCK inhibitor. In the preferred embodiments, the compositions comprise a SAH/PRC/EZH2 inhibitor, a HDAC inhibitor, L-ascorbic acid, an activator of JAK/STAT3 signaling, an inhibitor of MAPK/ERK signaling, and a tankyrase inhibitor, and optionally an activator of ACTIVIN/NODAL signaling and optionally a ROCK inhibitor. It should be understood that each of the above-mentioned components, when present in the composition, should be present in an amount that allows their respective amount in a culture medium containing the composition falling within their respective range of each culture medium as defined in any of the embodiments of the subject application. More preferably, using the composition could formulate the culture medium of any embodiment as described in the subject application.

In one or more preferred embodiments, the composition of the present application comprises DZNep or CPI-1205, and TSA or VPA or NaB, and optional L-ascorbic acid, optional LIF, optional PD0325901 and optional IWR1 or XAV939; preferably, each of the components is present in an amount that allows the culture medium containing the composition to comprise: 5 to 15 nM, preferably 10 nM, of DZNep, or 0.5 to 2 mM, preferably 1 mM, of CPI-1205; 3 to 6 nM, preferably 5 nM, of TSA, or 0.25 to 1 mM, preferably 0.5 mM, of VPA (VPA), or 0.25 to 1 mM, preferably 0.5 mM, of NaB; and optionally 40 to 90 μg/mL, preferably 50 μg/mL, of L-ascorbic acid, optionally 10 to 30 ng/mL, preferably 20 ng/mL, of LIF, optionally 0.5 to 1.5 μM, preferably 1 μM, of PD0325901, optionally 3 to 6 μM, preferably 5 NM, of IWR1 or XAV939. The compositions may further comprise ACTIVIN A or NODAL, and/or Y27632, thiazovivin or hydroxyfasudil, and/or an extracellular matrix, wherein each of the components is present in an amount that allows the culture medium containing the composition to comprise 10 to 25 ng/mL, preferably 20 ng/mL ACTIVIN A or NODAL, and/or 0.5 to 2 μM, preferably 1 μM, of Y27632, thiazovivin or hydroxyfasudil, and/or 0.1% to 0.5% (v/v) of an extracellular matrix.

In one or more embodiments, the composition of the present application comprises DZNep or CPI-1205, and TSA or VPA or NaB, and optional L-ascorbic acid, optional LIF, optional PD0325901 and optional IWR1 or XAV939; preferably, each of the components is present in an amount that allows the culture medium containing the composition to comprise: 40 to 70 nM, preferably 50 nM, of DZNep, or 2 to 4 mM, preferably 3 mM, of CPI-1205; 10 to 30 nM, preferably 20 nM, of TSA, or 0.5 to 1.5 mM, preferably 1 mM, of VPA, or 0.5 to 1.5 mM, preferably 1 mM, of NaB; and optionally 40 to 90 ng/mL, preferably 50 ng/mL, of L-ascorbic acid, optionally 10 to 30 ng/mL, preferably 20 ng/mL, of LIF, optionally 0.5 to 1.5 μM, preferably 1 μM, of PD0325901, optionally 3 to 6 μM, preferably 5 μM, of IWR1 or XAV939. The compositions may further comprises ACTIVIN A or NODAL, and/or Y27632, thiazovivin or hydroxyfasudil, and/or an extracellular matrix, wherein each of the components is present in an amount that allows the culture medium containing the composition to comprise 10 to 25 ng/mL, preferably 20 ng/mL ACTIVIN A or NODAL, and/or 0.5 to 2 μM, preferably 1 μM, of Y27632, thiazovivin or hydroxyfasudil, and/or 0.1% to 0.5% (v/v) of an extracellular matrix.

In some embodiments, kits may comprise the above-mentioned compositions.

Kits of the subject application may further comprise a culture medium for maintenance of PSCs, such as mTeSR1 or E8 medium, and/or medium for blastoid formation, such as REM medium (REM is a modified reconstructed embryo medium (Zhang Shaopeng et al. 2019)) supplemented with 8-15 μM Y27632 or devoid of Y27632. Reagents conventionally used in culture of stem cells may also be provided in the kits. Such reagents include but are not limited to PBS, EDTA solution, and/or TrypLE: 0.5 mM EDTA (1:1). Feeder cells and/or extracellular matrix can also be provided in the kits.

### IV. Methods and Uses

Culture media of the subject application can be used to reprogram primate somatic cells to ICLCs, to convert primate PSCs to ICLCs, and to convert primate PSCs or ICLCs to 8CLCs.

Therefore, one aspect of the present application discloses a method for reprogramming primate somatic cells to ICLCs, comprising culturing the somatic cells in a conversion culture medium comprising a SAH/PRC/EZH2 inhibitor, a HDAC inhibitor, L-ascorbic acid, an activator of JAK/STAT3 signaling, an inhibitor of MAPK/ERK signaling, and a tankyrase inhibitor, an optional activator of ACTIVIN/NODAL signaling and an optional ROCK inhibitor, with or without an extracellular matrix. The resultant ICLCs can be used in the method of converting ICLCs to 8CLCs. Preferably, the conversion cultures are those described in any embodiments of the present application.

Another aspect of the present application discloses a method for converting primate PSCs to ICLCs, or for converting primate PSCs or ICLCs to 8CLCs, comprising culturing the primate PSCs in a conversion culture medium comprising a SAH/PRC/EZH2 inhibitor, a HDAC inhibitor, L-ascorbic acid, an activator of JAK/STAT3 signaling, an inhibitor of MAPK/ERK signaling, and a tankyrase inhibitor, an optional activator of ACTIVIN/NODAL signaling and an optional ROCK inhibitor, with or without an extracellular matrix. In the preferred embodiments, the conversion culture medium is the culture medium as defined in any of the above-mentioned embodiments.

In one or more preferred embodiments, the method is a method for converting primate PSCs to ICLCs, and the conversion culture medium is the culture medium as defined in any of the above-mentioned embodiments with a relative lower concentration of the SAH/PRC/EZH2 inhibitor and the HDAC inhibitor.

In some other preferred embodiments, the method is a method for converting primate PSCs or ICLCs to 8CLCs and the conversion culture medium is the culture medium as defined in any of the above-mentioned embodiments with a relative higher concentration of the SAH/PRC/EZH2 inhibitor and the HDAC inhibitor.

Conventional conditions for culturing stem cells may be used to convert PSCs to ICLCs or 8CLCs. For example, single primed PSC may be plated in conventional culture medium, such as mTeSR1 or E8, optionally supplemented with 5 to 15 μM ROCK inhibitor, such as Y27632. After a period of culture, for example, 24 hours later, the medium is switched to the culture medium of the subject application and the cells are continued to be cultured until the desired ICLCs or 8CLCs are produced. During culture, the culture medium may be refreshed as necessary, preferably refreshed daily. For passaging, cells may be dissociated into single cells with conventional methods and then plated and cultured again with the culture medium of the subject application until ICLCs or 8CLCs are formed. It is preferred that cells are passaged as single cells every 3 to 4 days with a split ratio of 1:4 to 1:8, preferably 1:6 to 1:8, and generally, cells will be converted to ICLCs from primed PSC in approximately 2 weeks, will be converted to 8CLCs from primed PSC in about one week, and will be converted to 8CLCs from ICLCs in 3 to 5 days after culturing ICLCs with the culture medium containing a relatively higher concentration of the SAH/PRC/EZH2 inhibitor and the HDAC inhibitor. It should be understood that, ICLCs used for converting to 8CLC may be the ICLCs obtained by culturing primate PSC with any of the methods described herein, or may be the known ICLCs or the ICLCs prepared from any methods known in the art.

In general, cells may be cultured at 37° C. under a normoxic condition (5% CO₂) or a hypoxic condition (5% CO 2 and 5% O₂). There is not specific limitation on the time of culture, which can readily be determined by the skilled artisan based on the subject disclosure and the conventional techniques of the art. Plating concentration could be determined by the skilled artisan according to the common knowledge of the art and the actual production condition.

In some embodiments of the present application, cells may be cultured under one or more conditions selected from a group consisting of: (i) on feeder cells; (ii) on an extracellular matrix devoid of feeders; (iii) in suspension devoid of feeder cells; (iv) propagation in hypoxic or normoxic condition at about 37° C. temperature; (v) passaging as single cells every 3-4 days with a split ratio of 1:4 to 1:8; (vi) changing medium daily.

In some embodiments, for conversion primate PSCs to ICLCs, the single primed primate PSCs are plated on feeder in mTeSR1 or E8 medium supplemented with 5 to 15 μM ROCK inhibitor (such as Y27632) and cultured for a period of time, such as 24 hours, then the mTeSR1 or E8 medium is switched to the conversion culture medium of the subject application with relatively lower concentration of the SAH/PRC/EZH2 inhibitor and the HDAC inhibitor and cells are cultured under hypoxic or normoxic condition at about 37° C. temperature with the medium being refreshed daily. During culture, cells are passaged every 3 to 4 days as single cells with a split ratio of 1:4 to 1:8 until ICLCs are obtained. In some embodiments, the single primed primate PSCs are cultured as described above except plating the cells on about 1% (v/v) of an extracellular matrix, such as Geltrex™, in DMEM-F12 coated plates instead of on feeder cell.

In some embodiments, for conversion primate PSCs to ICLCs, the single primed primate PSCs are plated on plate using mTeSR1 or E8 medium supplemented with 5 to 15 μM ROCK inhibitor (such as Y27632) and cultured for a period of time, such as 24 hours, then the mTeSR1 or E8 medium is switched to the conversion culture medium of the subject application with relatively lower concentration of the SAH/PRC/EZH2 inhibitor and the HDAC inhibitor and cells are cultured under hypoxic condition; after forming small spheres, the spheres are transferred to flasks for suspension culture with medium being refreshed daily; wherein cells are passaged every 4 to 5 days as single cells with a split ratio of 1:4 to 1:8 until ICLCs are obtained.

In some embodiments, for conversion to 8CLCs from primate PSCs, single primed PSCs are plated on feeder using mTeSR1 or E8 medium supplemented with 5 to 15 μM ROCK inhibitor (such as Y27632) for a period of time, such as 24 hours, then the medium is switched to the conversion culture medium of the subject application which has a relatively higher concentration of SAH/PRC/EZH2 inhibitor and HDAC inhibitor and cells are cultured under hypoxic or normoxic condition with the medium being refreshed daily; wherein cells are passaged every 3 to 4 days as single cells with a split ratio of 1:4 to 1:8.

In some embodiments, for conversion to 8CLCs from ICLCs, single cells are dissociated from ICLCs and plated on feeders using the conversion culture medium of the subject application which has a relatively lower concentration of SAH/PRC/EZH2 inhibitor and HDAC inhibitor for a period of time, such as 24 hours, then the culture medium is switched to the conversion culture medium of the subject application which has a relatively higher concentration of SAH/PRC/EZH2 inhibitor and HDAC inhibitor and cells are cultured for 3 to 5 days without passaging, with the medium being refreshed daily.

In some embodiments, for conversion to 8CLCs from ICLCs, single cells are dissociated from ICLCs and suspended in conversion culture medium of the subject application which has a relatively lower concentration of SAH/PRC/EZH2 inhibitor and HDAC inhibitor for suspension culture for a period of time, wherein the conversion culture medium is supplemented with 5 to 15 μM ROCK inhibitor (such as Y27632); after forming small aggregates, medium is changed to the conversion culture medium of the subject application which has a relatively higher concentration of SAH/PRC/EZH2 inhibitor and HDAC inhibitor but without adding extra ROCK inhibitor (such as Y27632) for conversion for several days without passaging, with the medium being refreshed daily.

Uses of any of the conversion culture medium described in any of the embodiments of the present application in reprogramming primate somatic cells to ICLCs, in conversion of primate PSCs to ICLCs, or in conversion of primate PSCs or ICLCs to 8CLCs, or in manufacture of a culture medium or a kit reprogramming primate somatic cells to ICLCs, or for converting primate PSCs to ICLCs, or for converting primate PSCs or ICLCs to 8CLCs, are also included in the subject application.

In some embodiments, the subject application also comprises use of a SAH/PRC/EZH2 inhibitor and a HDAC inhibitor in the manufacture of a culture medium or a kit reprogramming primate somatic cells to iPSCs, or for converting primate PSCs to ICLCs, or for converting primate PSCs or ICLCs to 8CLCs. Preferably, the culture medium or the kit may further comprise one or more components selected from a group consisting of L-ascorbic acid, an activator of JAK/STAT3 signaling, an inhibitor of MAPK/ERK signaling, and a tankyrase inhibitor, and an optional activator of ACTIVIN/NODAL signaling and an optional ROCK inhibitor (such as Y27632), and optional an extracellular matrix.

In some other embodiments, the methods for converting primate PSCs to ICLCs, and converting primate PSCs or ICLCs to 8CLCs may comprise a genetically engineering step to reduce the activity of SAH, PRC and/or EZH2 of the PSCs, and/or to reduce the activity of HDAC of the cells, by knockdown and/or knockout of one or more relevant genes in the cells, before culturing the primate PSCs with the culture medium of the subject application. Preferably, to reduce the activity of SAH, PRC and/or EZH2 of the PSCs, expression of any of the SAH, PRC and EZH2 regulators can be knocked down by, such as a siRNA technique, or any of the genes can be knocked out by, such as CRISPR/Cas9 technique. Similarly, the expression of HDAC regulators can be knocked down or knocked out by the same above-mentioned means. After knocking down or knocking out, the resultant cells can be cultured in any of the culture media of the subject application in accordance with the aforementioned methods. In some embodiments, when the activity of SAH, PRC and/or EZH2 of the PSCs is reduced, the culture medium used for culturing the genetically engineered primate PSCs may or may not contain the SAH/PRC/EZH2 inhibitor. Similarly, if the activity of HDAC of the PSCs is reduced, the culture medium may or may not contain the HDAC inhibitor. In the case that both the activity of SAH, PRC and/or EZH2 and the activity of HDAC are reduced, the culture medium may contain neither the SAH/PRC/EZH2 inhibitor nor the HDAC inhibitor, or may contain either the SAH/PRC/EZH2 inhibitor or the HDAC inhibitor.

Therefore, in some embodiments, the subject application further provides culture mediums comprising neither the SAH/PRC/EZH2 inhibitor nor the HDAC inhibitor, or comprising either the SAH/PRC/EZH2 inhibitor or the HDAC inhibitor, with other components and amounts identical to any of the above-mentioned embodiments for culture medium in Part II. In some embodiments, the culture medium may contain reagents for liposome transfection. For example, in the above mentioned methods, the primate PSCs are cultured in a culture medium containing vectors for expression snRNA directed to, such as any of the SAH, PRC and EZH2 regulators, and reagents for liposome transfection for transfecting the vector into the PSCs for genetically engineering, in addition to other components described in the culture medium described in Part II, and the culture medium may or may not contain the SAH/PRC/EZH2 inhibitor.

### V. Biological Function of STELLA

STELLA is a DNA methylation regulator. Its ectopic over-expression in somatic cells can induce comprehensive DNA demethylation by interfering with the function of UHRF1, a DNA methylation regulator. The dysfunction of UHRF1 caused by STELLA deletion would lead to the accumulation of abnormal DNA methylation during oogenesis (Li et al., 2018). It is also documented that STELLA maintains maternal imprinting by protecting 5 mC from Teti-mediated conversion to 5 hmC at specific loci (Nakamura et al., 2012).

In the subject application, the inventors first discover that STELLA knock out hinders the induction of ICLCs and 8CLCs. Therefore, STELLA is necessary for controlled DNA demethylation in conversion process. The inventors also find that adding SAH/PRC/EZH2 inhibitors promotes induction of ICLCs and 8CLCs through rewiring histone modification and DNA methylation landscape.

Therefore, in some embodiments, the subject application further comprises use of an agent which can promote expression of STELLA or improve activity of STELLA in the manufacture of a reagent, a culture medium or a kit for promoting conversion of primate PSCs to ICLCs, or for promoting conversion of primate PSCs or ICLCs to 8CLCs, and use of an agent which can promote expression of STELLA or improve activity of STELLA for promoting conversion of primate PSCs to ICLCs, or for promoting conversion of primate PSCs or ICLCs to 8CLCs.

Methods for promoting conversion of primate PSCs to ICLCs, or conversion of primate PSCs or ICLCs to 8CLCs are also provided, which comprises culturing the primate PSCs in the presence of an effective amount of an agent which can promote expression of STELLA or improve activity of STELLA. The effective amount of the agent can readily be determined by the skilled artisan of the art based on the disclosure of the subject application and the teaching of the prior art.

In some preferred embodiments, the agent which can promote expression of STELLA or improve activity of STELLA is an inhibitor of SAH/PRC/EZH2, which includes but is not limited to DZNep and CPI-1205. The SAH/PRC/EZH2 inhibitors can be used alone or in combination, generally in their respective conventional amounts which will not lead to cell death. For example, DZNep can be used in the media at a final concentration of from 5 to 80 nM, preferably 5 to 50 nM, and CPI-1205 can be used in the media at a final concentration of 0.5 to 5 mM, preferably 1 to 3 mM. In one or more embodiments, the SAH/PRC/EZH2 inhibitor is commonly known as a PRC inhibitor.

In some further preferred embodiments, methods for promoting conversion of primate PSCs to ICLCs comprise culturing the primate PSCs in the presence of 5 to 15 nM, preferably 10 nM, of DZNep, or 0.5 to 2 mM, preferably 1 mM CPI-1205. In some other preferred embodiments, methods for promoting conversion of primate PSCs or ICLCs to 8CLCs comprise culturing the primate PSCs in the presence of 40 to 70 nM, preferably 50 nM, of DZNep, or 2 to 4 mM, preferably 3 mM CPI-1205.A SAH/PRC/EZH2 inhibitor for use in a method for promoting conversion of primate PSCs to ICLCs, or conversion of primate PSCs or ICLCs to 8CLCs are also included in the subject application.

### VI. Cells

The subject application also provides isolated primate ICLCs. The ICLCs of the present application have transcriptome close to human preimplantation ICM, have transposable element profile close to human preimplantation ICM, have DNA methylome close to human preimplantation ICM, have chromatin landscape close to human preimplantation ICM, and have metabolic state close to human preimplantation ICM.

As used herein, the term “close to” is intended to mean “substantially identical” or “without substantial difference”. The skilled artisan of the art is able to acknowledge, based on the common knowledge of the art, that the cells of the subject application, including cells from ICLC or from 8CLC of the present application, are substantially identical to the native ICM cells or 8C embryo cells, even though there may have some minor differences.

Preferably, the ICLCs of the present application exhibit significantly higher expression level of preimplantation ICM markers, including KLF17, DNMT3L, DPPA5, STELLA, TFCP2L1, MAEL, and REX1. More preferably, the expression level of at least one of the above-mentioned preimplantation ICM markers in ICLCs of the present application is 10 or more times higher than the expression level of that corresponding preimplantation ICM markers in primed human PSCs; preferably the expression level of all the above-mentioned preimplantation ICM markers in ICLCs of the present application is 10 or more times of the expression level of the corresponding preimplantation ICM marker in primed human PSCs.

Preferably, the ICLCs of the present application are further characterized by one or more of the following characteristics:
1) being able to self-renew and maintain pluripotency in culture;
2) maintaining genomic stability in culture according to karyotype;
3) being able to give rise to cells of the 3 germ layers;
4) being able to give rise to primordial germ cell-like cells;
being able to integrate to mouse embryo and contribute to embryonic and extraembryonic tissues;
6) being able to transit to extraembryonic cell fate in vitro; and
7) being able to form blastocyst-like structures in vitro.

Such ICLCs can be obtained by culturing primate PSCs by any of the methods as described in any of the embodiments of the subject application. Therefore, in some embodiments, the subject application also includes cells, specifically, ICLCs, obtained by any of the methods described herein.

The subject application also provides isolated 8CLCs, which express 8C state specific markers, including ZSCAN4, TPRX1, ZIM3, ZSCAN5B, ZNF280A and ARGFX, at a level substantially higher than cells of preimplantation ICM-like state or primed state. Preferably, at least one of the specific markers exhibits an expression level which is 5 or more times higher than the expression level of the corresponding 8C specific marker in primed PSCs or ICLCs. Preferably, all the above-mentioned specific markers exhibit an expression level which is 5 or more times higher than that the expression level of the corresponding 8C specific markers in primed PSCs or ICLCs.

Preferably, the 8CLCs of the present application have transcriptome, transposable element profile, and chromatin landscape close to human 8C stage embryos. More preferably, the 8CLCs of the present application are further characterized by one or more of the following characteristics:
1) maintaining genomic stability in culture according to karyotype;
2) being able to give rise to cells of the 3 germ layers;
3) being able to give rise to primordial germ cell-like cells;
4) being able to integrate to mouse embryos and contribute to embryonic and extraembryonic tissues;
5) being able to transit to extraembryonic cell fate in vitro; and
6) being able to form blastocyst-like structures in vitro.

ICLCs obtained by reprogramming of somatic cells with the conversion culture medium of the present application are also contemplated in the subject application.

Cell cultures containing the cells of the present application, especially the ICLCs and/or the 8CLCs of the present application, are also contemplated in the present application. Culture medium described in any of the subject application can also be included in the cell cultures.

The subject invention will be described in the following non-limiting examples. It should be understood that these examples are only for illustrative purpose, but not for limiting the scope of the invention in various manner. Various variations, and modifications may be made within the spirit of the present application. The technologies involved, unless otherwise specified, are conventional technologies in various fields, such as molecular biology, cell biology, biochemistry, etc., which are well known to those skilled in the art.

### Example 1

Materials and Methods

4CL Basal Medium

1:1 mix of Neurobasal Medium (Gibco) and Advanced DMEM/F12 (Gibco), supplemented with N2 supplement (1X, Gibco), B27 supplement (1X, Gibco) (homemade N2 and B27 can be used), Sodium Pyruvate (1X, Hyclone), Non-Essential Amino Acid (NEAA) (Gibco), Glutamax™ (1X, Gibco) and Penicillin-Streptomycin (1X, Gibco).

4CL Supplements

4CL Medium 1, Supplemented in the 4CL Basal Medium with:

SAH/PRC/EZH2 inhibitor (10 nM DZNep), MAC inhibitor (5 nM TSA), L-ascorbic acid (50 μg/mL), JAK/STAT3 activator (20 ng/mL human LIF), MAPK/ERK inhibitor (1 μM PD0325901), tankyrase inhibitor (5 μM IWR1), ACTIVIN A/NODAL activator (20 ng/mL human ACTIVIN A), extracellular matrix (0.2% (v/v) Geltrex™), optional ROCK inhibitor (1 μM Y27632). Catalogues for these reagents and their substitutes are listed in Table 1.

TABLE 1

| Reagent name | Brand | Catalog |
|---|---|---|
| DZNep | Selleck | S7120 |
| CPI-1205 | Selleck | s8353 |
| Trichostatin A | Sigma | V900931-5MG |
| VPA | Calbiochem | 6676380 |
| Sodium butyrate | Sigma | 303410-100G |
| L-ascorbic acid 2-phosphate | Sigma | A8960-5G |
| Recombinant Human LIF | Peprotech | 300-05 |
| PD0325901 | Axon | 1408 |
| IWR1 | Sigma | I0161-25MG |
| Human Activin A | Peprotech | 120-14E |
| Recombinant Human Nodal Protein | R&D | 3218-ND-025 |
| XAV939 | Selleck | S1180 |
| Geltrex ™ LDEV-Free, | Invitrogen | A1413302 |
| hESC-Qualified, Reduced Growth | (ThermoFisher | |
| Factor Basement Membrane Matrix | Scientific) | |
| Matrigel ™ | BD | 354277 |
| Y27632 | Axon | 1683 |
| Thiazovivin | Axon | 1535 |
| Hydroxyfasudil (HA-1100) HCl | Selleck | S8208 |

REM Medium

1:1 mixture of Advanced DMEM F12 (Gibco) and RPMI 1460 (Gibco), supplemented with 17.5% Fetal Bovine Serum (NATOCOR), 1X Glutmax™ (Gibco), 1X NEAA (Gibco), 1X Sodium Pyruvate (Hyclone), and 1X Penicillin-Streptomycin (Gibco). REM is a modified reconstructed embryo medium (Zhang Shaopeng et al. 2019).

Cells

H9 Human ESC Line.

Procedures:

1) Maintenance of Primed Human PSCs

All provided human PSCs were routinely maintained on Matrigel™ or Geltrex™ coated plates in mTeSR1 or E8 medium. Generally, cells were passaged every 4 to 5 days with 0.5 mM EDTA. For passaging, cells were washed with PBS once and treated with 0.5 mM EDTA for 5 mins. Then, EDTA was removed and cells were detached as small clumps using a Pasteur pipette with mTeSR1 or E8 medium. Primed human PSCs were grown in an incubator under normoxic conditions (37° C., 5% CO₂).

2) Conversion to ICLCs on Feeders

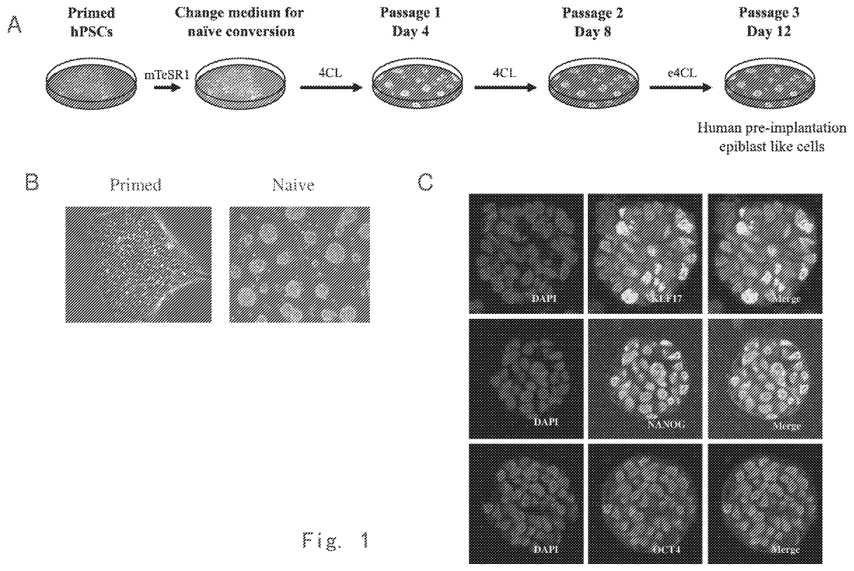

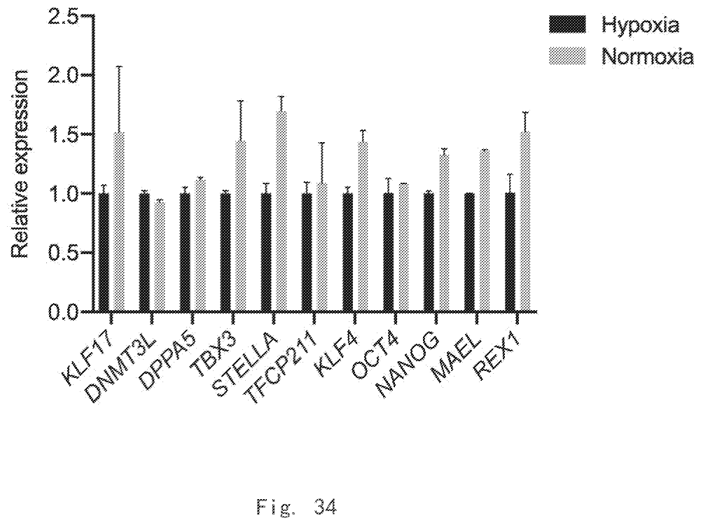

One day before initiation of conversion, primed human PSCs were washed with PBS once and dissociated into single cells and plated at a density of 1,000 to 1,500 cells/cm 2 on feeder in mTeSR1 or E8 medium supplemented with 10 μM Y27632. Twenty-four hours later, culture medium was switched to 4CL medium 1. The culture medium was refreshed with the same medium every 24 hours. Colonies became round and domed-shape in 24 to 48 hours. Cells were passaged every 3 to 4 days. For passaging, cells were dissociated into single cells using TrypLE: 0.5 mM EDTA (1:1) and plated at a density of 1,000 to 1,500 cells/cm 2 on feeder (feeders were seeded on Geltrex™/Matrigel™ pre-treated plate) (FIG. 1). The ICLCs induction and maintenance can be conducted under hypoxic condition (37° C., 5% CO₂, 5% O₂), or normoxic condition (37° C., 5% CO₂, 21% O₂) (FIG. 34), preferably hypoxia condition.

3) Blastocyst-like structure (also termed blastoid) formation Primed human PSCs or ICLCs were digested into single cells and filtered with 40 μm strainers. The cell number was counted with haemocytometer. The well of 24 well plate was coated with 200 μl thawed Geltrex and placed into incubator at 37° C. for 7 mins to form semi-solid matrix. For each well, 30,000 cells were resuspended evenly into 500 ul REM medium for blastoid formation, which was supplemented with 10 μM Y27632. Afterwards, the cell mixture was plated onto semi-solid Geltrex and put back into incubator and incubated under 37° C., 5% CO₂. Twenty-four hours later, the medium was replaced with REM medium supplemented with 4% (v/v) Geltrex devoid of Y27632. The culture medium was refreshed daily, cells were cultured at 37° C. in hypoxia condition (5% CO₂, 5% O₂).

Experimental Results

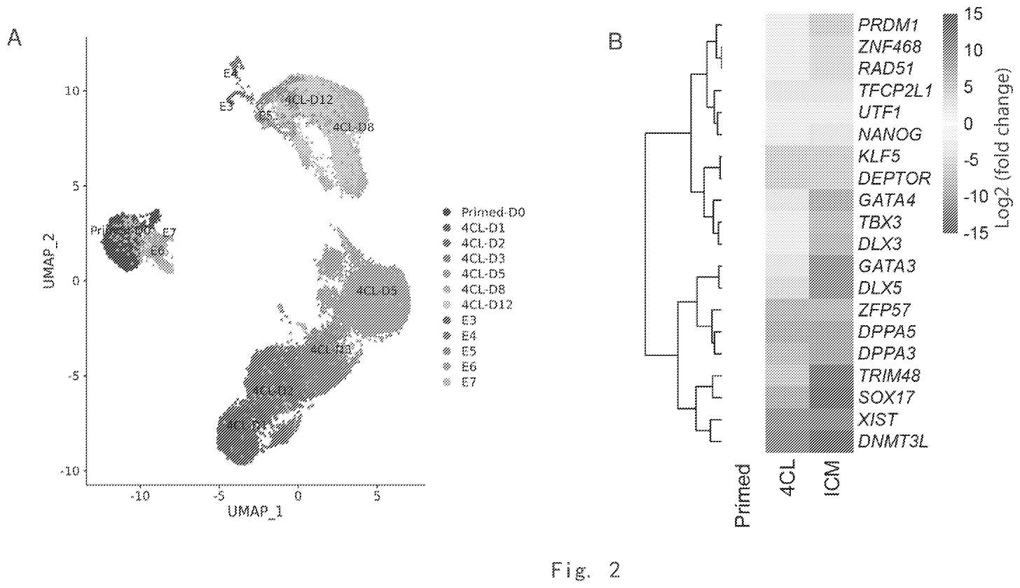

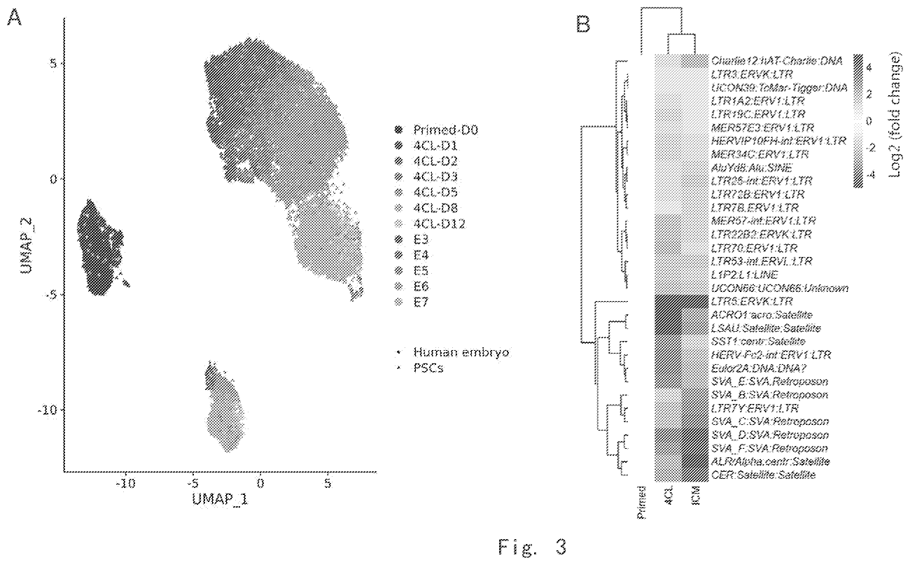

FIG. 1(A) is a schematic presentation of ICLCs induction from primed human PSCs. FIG. 1(B) shows a representative image of colony morphology under phase contrast microscope of primed human PSCs (left panel) and ICLCs (right panel). The flat primed human PSCs become domed-shape ICLCs after conversion. FIG. 1(C) are RT-qPCR and immunostaining data demonstrating that a panel of preimplantation ICM markers KLF17, DNMT3L, DPPA5, STELLA, TFCP2L1, KLF4, MAEL, and REX1 are significantly induced in ICLCs compared with primed human PSCs cells. FIG. 1(D) showed that ICLCs induced under normoxia or hypoxia conditions have similar preimplantation ICM marker gene expression level. To characterize gene expression profile of ICLCs at single cell level, the inventors have applied single-cell RNA-Seq (scRNA-seq) for cells at the primed stage (Primed-DO) and then at day 1, 2, 3, 5, 8 and 12 after being cultured in 4CL medium 1 (4CL-D1/2/3/5/8/12). FIG. 2(A) is a 2D scatter plot of UMAP analysis for the cells at different time points together with published scRNA-seq data of in vivo human embryos at embryonic day 3, 4, 5, 6 and 7 (E3/4/5/6/7) (from E-MTAB-3929). It illustrates that conventional human PSCs in 4CL medium gradually acquire gene expression profile similar to human embryonic day 5 cells, which correspond to early preimplantation blastocyst stage. FIG. 2(B) is a heatmap using bulk RNA-seq of primed human PSCs, ICLCs and human preimplantation ICM cells (GSE101571), showing the expression level of known ICM markers in ICLCs are up-regulated to the level of ICM cells. It is known in the art that subgroups of TEs such as SVA_D is specifically activated from 8C to preimplantation ICM stage of human embryo. To investigate the activated TEs in ICLCs, the inventors have extracted TE profile from scRNA-seq data mentioned in FIG. 2(A). FIG. 3(A) is a 2D scatter plot of UMAP analysis for TE expression profile in cells at the primed stage (Primed-DO) and then at days 1, 2, 3, 5, 8 and 12 (D1/2/3/5/8) after being cultured in the 4CL medium 1 and human embryo cells at embryonic days 3, 4, 5, 6 and 7 (E3/4/5/6/7) (from E-MTAB-3929). It depicts that primed human PSCs in 4CL medium gradually capture TE profiles similar to cells of human embryo at embryonic day 4 (morula stage) and 5 (blastocyst stage). FIG. 3(B) further illustrates that the expression levels of multiple TE subgroups in ICLCs are induced to that of human preimplantation embryos (from GSE101571).

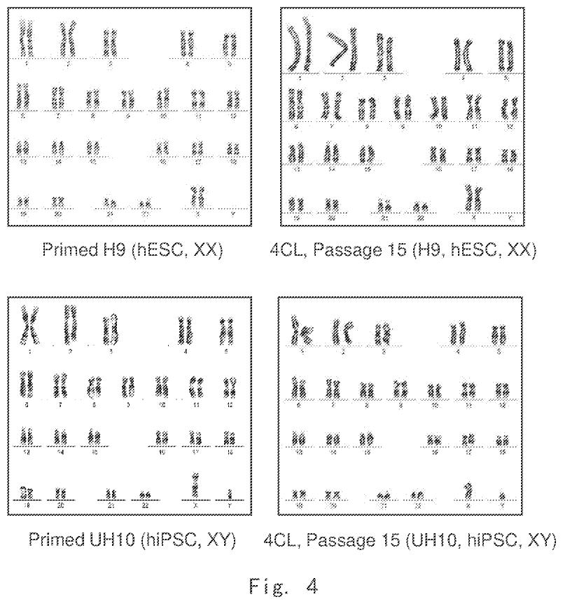

FIG. 4 illustrates that ICLCs maintain normal karyotype after prolonged culture (tested at passage 15, around 60 days in 4CL medium 1). One female human ESCs line (H9) and one male human iPSCs line (UH10) are shown. These results indicate that ICLCs derived by 4CL medium 1 gain preimplantation ICM-like gene expression profile and maintain stable genome after extended culture.

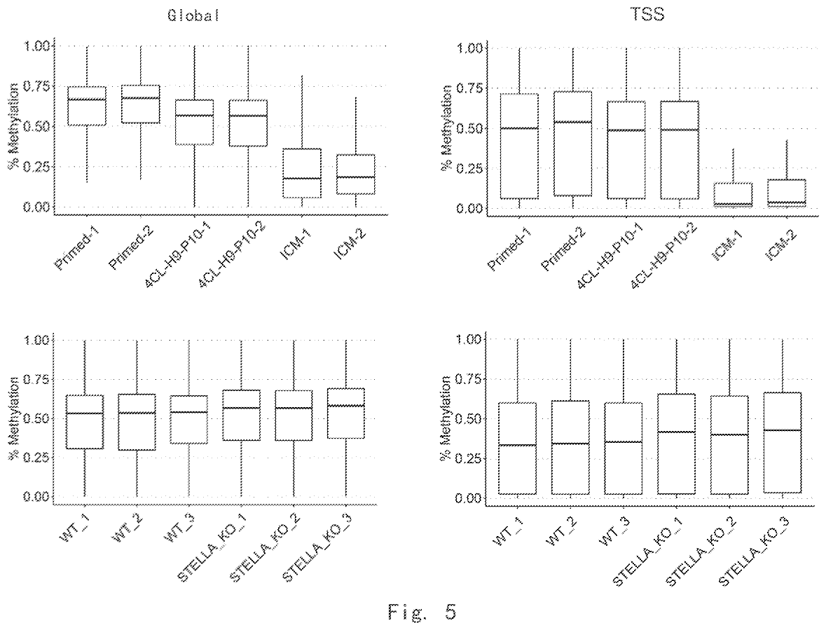

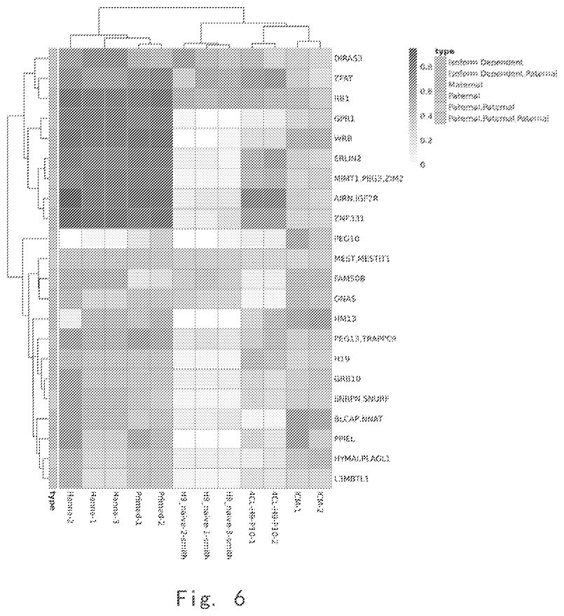

In epigenetic landscape, genome of preimplantation ICM is hypomethylated and the chromatins is more open compared to post-implantation ICM. To determine the impact of 4CL medium 1 on DNA methylation status, the inventors conducted reduced representation bisulphite sequencing (RRBS) for ICLCs and primed human PSCs. FIG. 5 is box plots showing the CpG DNA methylation across the whole genome (up left panel) is substantially reduced in ICLCs compared to primed human PSCs, whereas the methylation status of TSS shows slight difference (up right panel). Of note, the reduction of DNA methylation levels in global is prevented by knocking out STELLA (down left panel). FIG. 6 shows that the imprinting status of ICLCs are maintained similar to ICM.

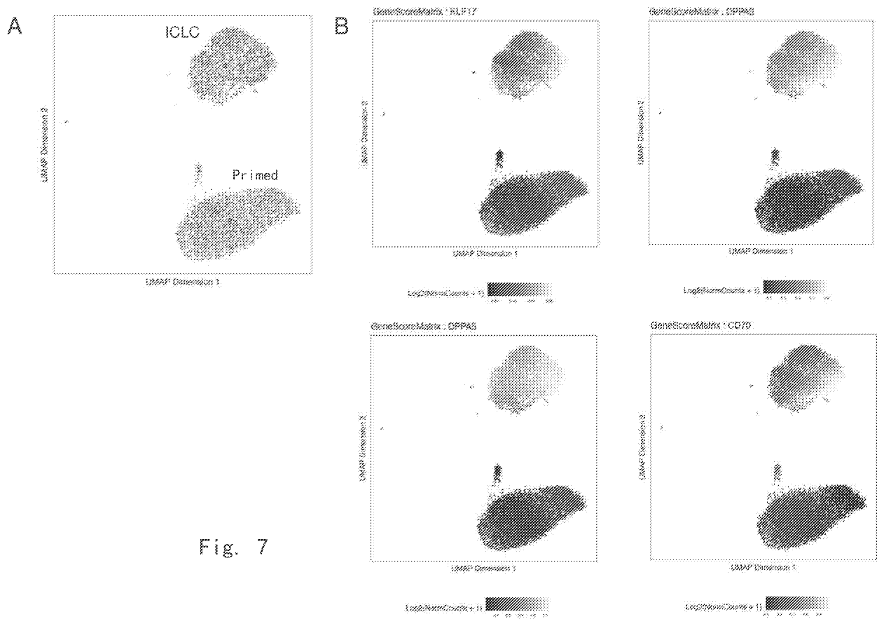

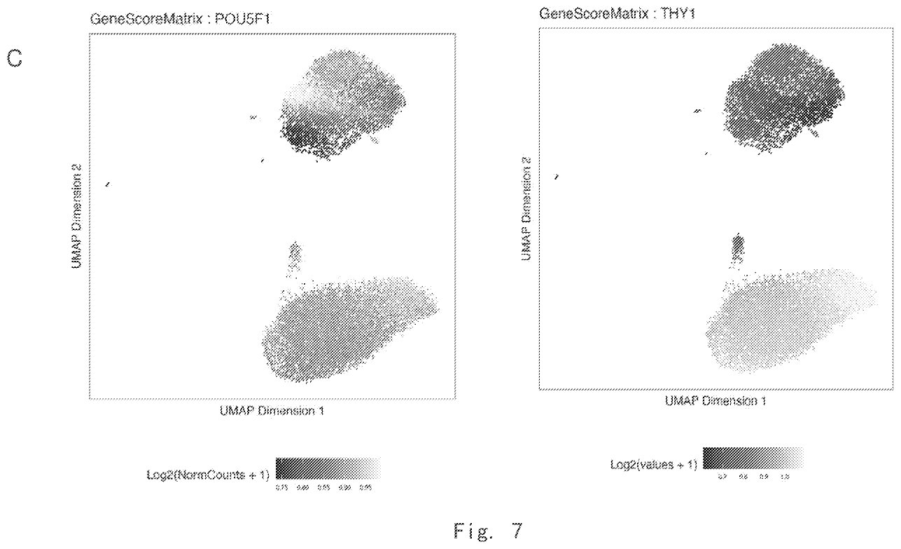

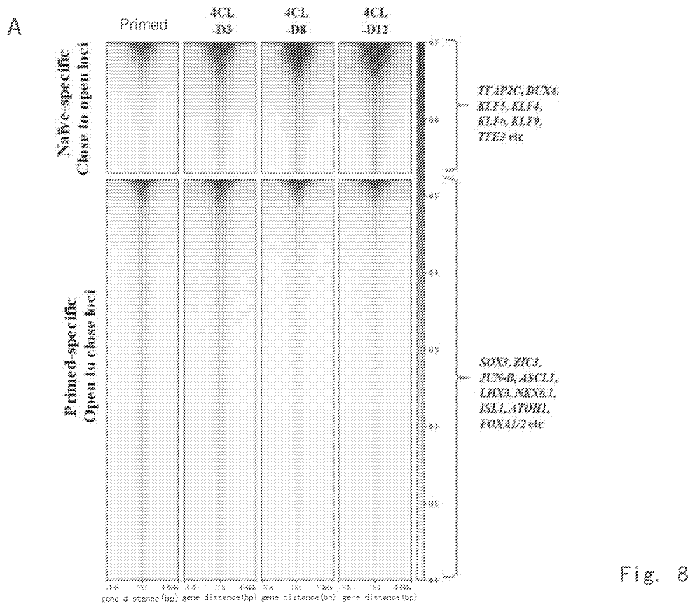

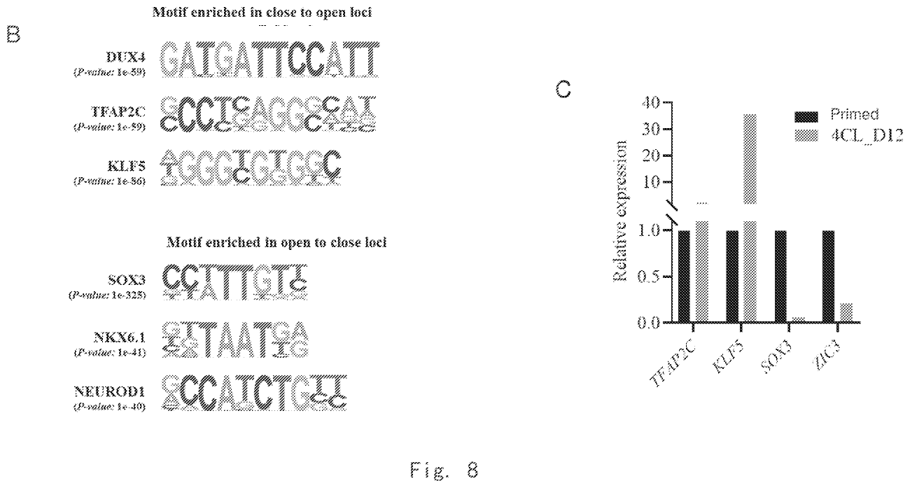

To determine the chromatin accessibility of ICLCs, the inventors performed single-cell ATAC-seq (scATAC-seq) and bulk ATAC-seq. FIG. 7(A) shows clear separation of primed human PSCs and ICLCs chromatin accessibility at single cell level. FIG. 7(B) shows the loci of preimplantation ICM specific genes, such as KLF17, STELLA, DPPA5, CD70, are largely open in ICLCs. FIG. 7(C) shows shared pluripotency genes like POU5F1 retained similar chromatin openness status between primed human PSCs and ICLCs while post-implantation specific genes become close, like THY1 (FIG. 7, D). Time course bulk ATAC-seq in FIG. 8 shows the stepwise change of chromatin accessibility during the conversion of primed human PSCs to ICLCs. Preimplantation specific loci (such as TFAP2C, KLF5, and TFE3) which are close in primed human PSCs open gradually during conversion, while post-implantation specific loci (such as ZIC3 and FOXA2) which are open in primed human PSCs close gradually (FIG. 8, A). Motif enrichment analysis shows that close to open regions may be bound by the preimplantation ICM specific transcription factors such as DUX, TFAP2C, and KLF5 (FIG. 8, B, upper panel), whereas the open to close regions may be bound by the post-implantation related transcription factors such as SOX3, NKX6.1, and NEUROD1 (FIG. 8, lower panel). FIG. 8(C) shows the correlation of gene expression and chromatin accessibility. These results demonstrate that 4CL medium 1 successfully rewires epigenetic landscape towards preimplantation ICM.

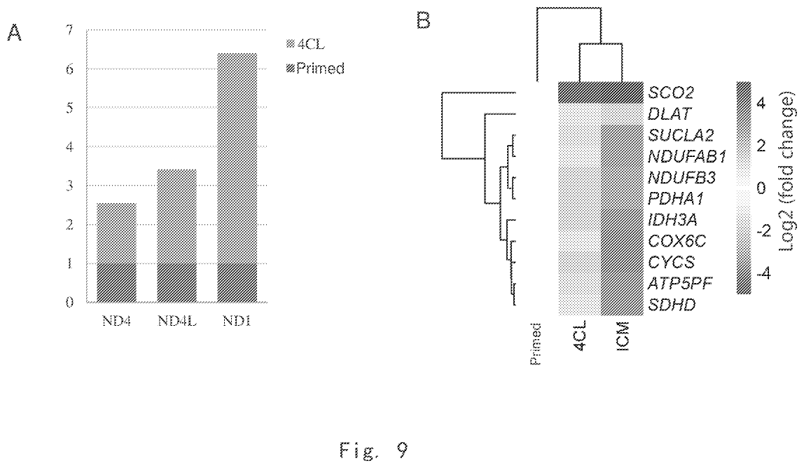

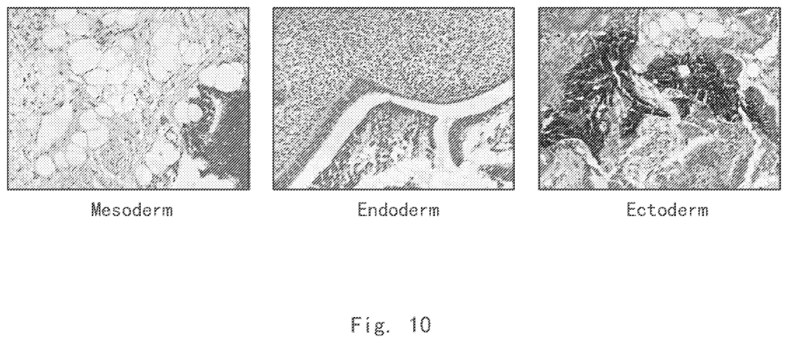

The inventors further investigated the metabolic state of ICLCs induced in 4CL medium 1. Preimplantation ICM mainly depends on oxidative phosphorylation (OxPhos) as their energy source, while predominantly depends on glycolysis after implantation. FIG. 9 show expression level of genes related to oxidative phosphorylation is substantially upregulated in ICLCs compared to primed human PSCs. These results imply oxidative phosphorylation is activated in ICLCs.

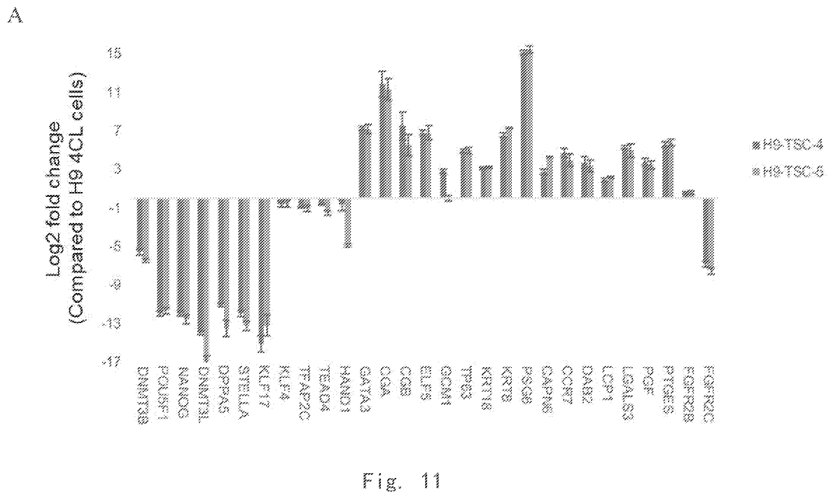

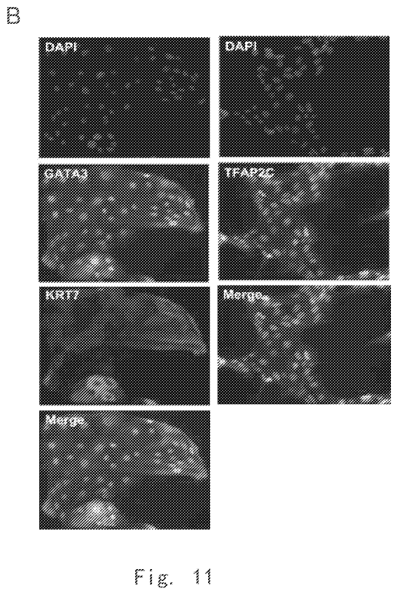

To determine the differentiation potential of ICLCs, the inventors performed teratoma formation assay using nude mice as receptor animal. FIG. 10 shows representative images of hematoxylin and eosin stained teratoma tissues formed 2 months after subcutaneously injection of 1 million ICLCs. They showed the presence of cells from all three germ layers: mesoderm (left panel), endoderm (middle panel) and ectoderm (right panel). It is known in the art that human ICLCs are capable to give rise to trophectoderm. Thus, the inventors induced trophoblast stem cell (TSC) from ICLCs using a previously published protocol. As shown in FIG. 11(A), multiple TSC markers such as GATA3, CGA, ELF5, TP63, KRT18, KRT8, PSG6, and CCR7 are significantly elevated in TSCs compared to undifferentiated ICLCs. FIG. 11(B) is immunofluorescence images showing expression of known TSC markers: GATA3, TFAP2C and KRT7. FIG. 11(C) is scatter plots of principal component analysis (PCA) showing that transcriptome of ICLCs derived TSCs are closer to human placenta choriocarcinoma cell line JEG3 and BeWo compared to ICLCs and placental cells (EGFR and HLAG). FIG. 11(D) shows DNA methylation status at the ELF5 promoter region of ICLCs-derived TSCs and other cell types. These results demonstrate that ICLCs gain developmental potential equivalent to human preimplantation embryo.

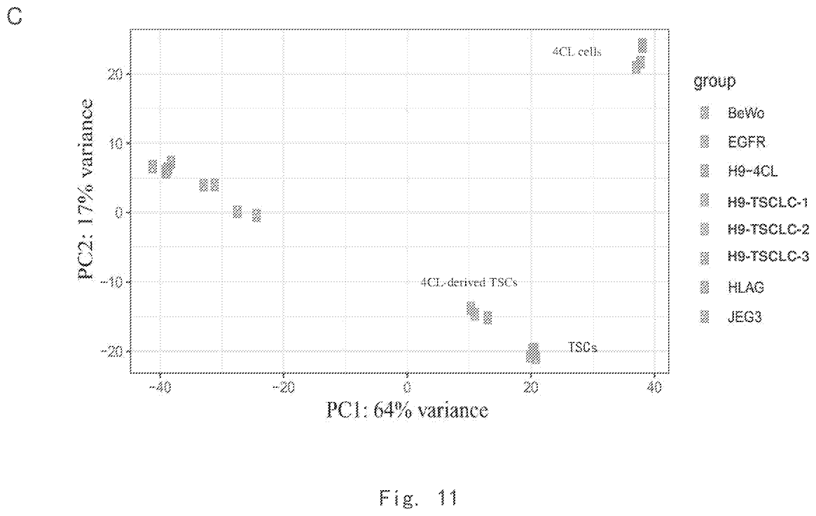

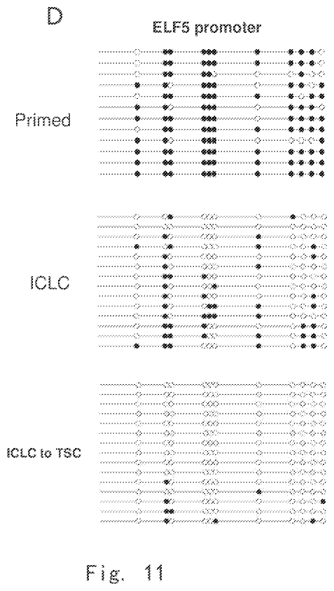

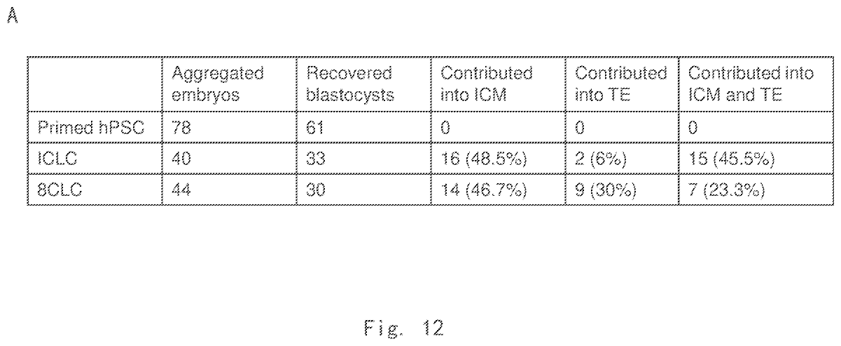

Due to ethical concerns, developmental potential of ICLCs cannot be tested using human embryos. Therefore, the inventors performed cross-species chimeric experiments by aggregating ICLCs with mouse 8C stage blastomeres. Human ICLCs are found successfully integrated into most mice embryos and formed chimeric blastocysts when checked after twenty-four hours of in vitro culture (FIG. 12, A-C). At this stage, human ICLCs are positioned in both ICM and TE parts of the chimeric blastocysts. FIG. 12(A) is the summary of chimera assay using DsRed labelled primed human PSCs and DsRed labeled ICLCs at blastocyst stage. FIG. 12(B) is representative images showing phase contrast (left) or red fluorescence channel (right) of blastocysts developed from mouse 8C blastomeres aggregated with DsRed labeled primed human PSCs (upper) or DsRed labeled ICLCs (lower). FIG. 12(C) is immunofluorescence of chimeric blastocysts stained with anti-OCT4 (ICM, green), anti-CDX2 (TE, grey), red signal is from integrated DsRed labeled ICLCs, and DAPI (blue) is used as nuclear counterstain. When these chimeric blastocysts were transferred into uterus of pseudo-pregnant mice and left to develop until embryonic day 10.5 (E10.5), the human cells can develop together with mice embryos and contribute into different tissues including embryonic tissues, extraembryonic placenta and yolk sac, as shown by microscope images in FIG. 13. FIG. 13(A) is representative images showing phase contrast (upper) or red fluorescence channel (lower) of E10.5 chimeric embryos (left), placenta (middle) or yolk sac (right). FIG. 13(B) is immunofluorescence images showing that hN (green) stained human cells differentiated into GATA6 (red) positive endodermal tissue. FIG. 13(C) is immunofluorescence images showing that DsRed-labelled human cells (red) differentiated into placental tissue as marked by GATA3 (green). Taken together, these results demonstrate that ICLCs can robustly integrate into mouse blastocysts and contribute into mouse E10.5 embryonic and extraembryonic tissues in vivo.

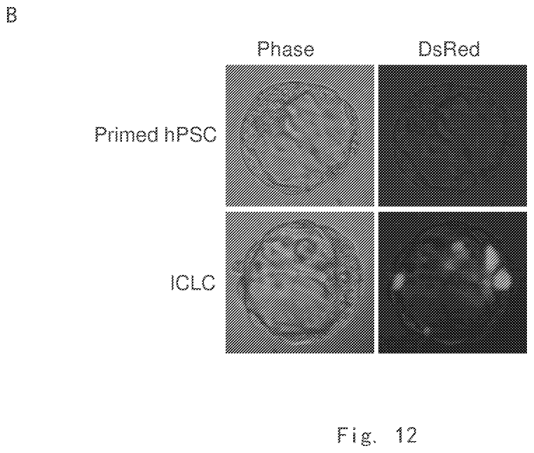

Recently, blastocyst-like structures (termed as blastoids) were generated from mouse extended PSCs (Li et al., 2019). However, such models using human cells have not yet been well studied. When applied ICLCs to an extracellular matrix rich medium, the inventors observed that blastocyst-like structures were developed solely from ICLCs, but not primed human PSCs (FIG. 14, A-B). FIG. 14(A) shows the morphology of blastoids generated from ICLCs in REM medium. FIG. 14(B) is immunofluorescence images of self-forming blastoids stained with anti-OCT4 (ICM, red), anti-GATA3 (1E, green) antibodies, or counterstained with DAPI (blue).

### Example 2

Materials and Methods

4CL Basal Medium

Same as Example 1.

4CL Supplements

Same as Example 1.

Cells

Human ESC lines: H1 (male), HN10 (female), HUES1 (male), and WIBR3 (female); human iPSC lines: CBC14 (generated by the inventors, female), C11 (generated by the inventors, female), Phoenix (a gift from Ulrich Martin's lab, female), DiPS 1016SevA (purchased from Harvard Stem Cell Institute, male), STiPS O-XX1 (purchased from Harvard Stem Cell Institute, female), UH10 (male).

Procedures

Using the Same Procedures of Example 1.

Experimental Results

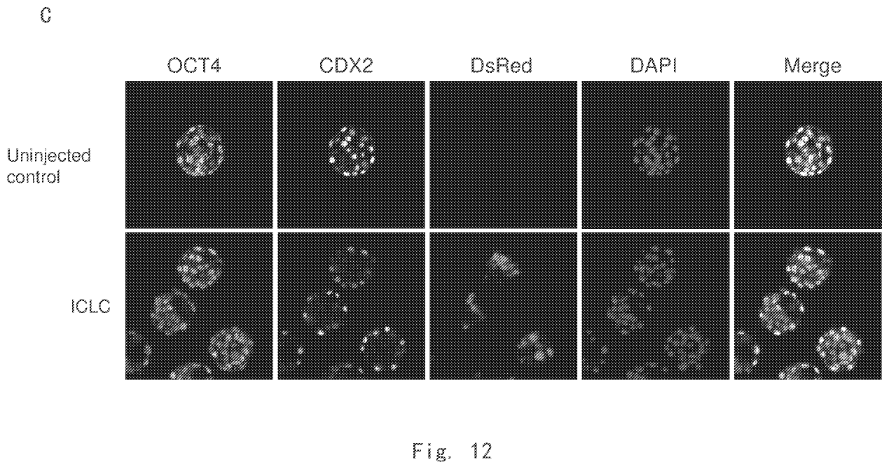

FIG. 15 is a bar chart of RT-qPCR data showing that preimplantation ICM markers KLF17, DNMT3L, DPPA5, STELLA, TFCP2L1, KLF4, MAEL, and REX1 are significantly induced in ICLCs converted from multiple primed human PSC lines. It demonstrates that 4CL medium 1 is commonly applicable to human PSCs.

### Example 3

Materials and Methods

4CL Basal Medium

Same as example 1.

4CL Supplements

Same as example 1.

Cells

H9 human ESC line.

Procedures

Using the same procedures of example 1, except plating cells on 1% (v/v) Geltrex™ in DMEM-F12 (cat #) coated plates instead of on feeder cells.

Experimental Results

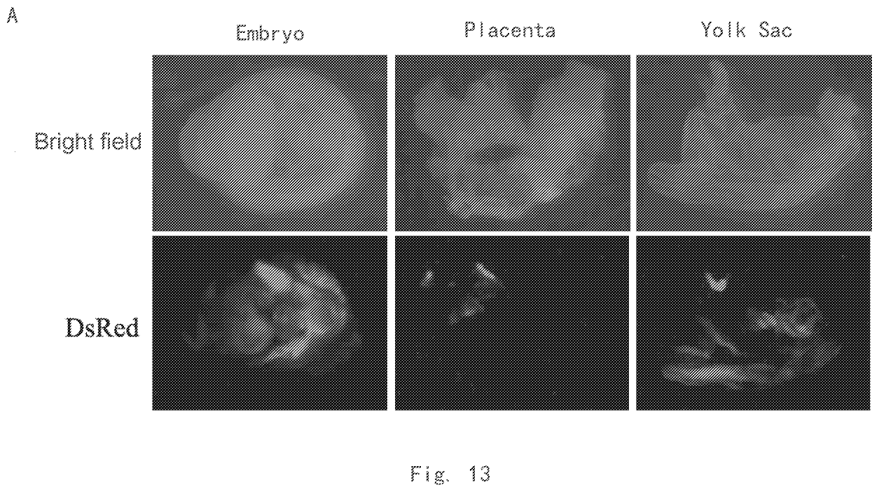

FIG. 16 is a bar chart of RT-qPCR data showing that preimplantation ICM markers KLF17, DNMT3L, DPPA5, STELLA, TFCP2L1, KLF4, MAEL, and REX1 are significantly induced in ICLCs converted on Geltrex™ coated plates using 4CL medium 1, which is similar to ICLCs on feeder. It indicates that 4CL medium 1 is also effective without feeder cells.

### Example 4

Materials and Methods

4CL Basal Medium

Same as example 1.

4CL Supplements

Same as example 1.

Cells

H9 human ESC line.

Procedures

Primed human PSCs were cultured following the same procedures of example 1. One day before initiation of the conversion, primed human PSCs were dissociated into single cells and plated 60,000 cells/well in Aggrewell™ 800 plates using mTeSR1 or E8 medium supplemented with 10 μM Y27632. Twenty-four hours later, culture medium was changed into 4CL medium 1, culture condition was then switched to hypoxic condition. Cells formed small spheres in 3 days. The spheres were then lifted and transferred to flasks (Greiner Bio-One, 658190) for suspension culture. Medium was refreshed daily. Cells were passaged every 4 to 5 days. For passaging, cells were dissociated into single cells using TrypLE: 0.5 mM EDTA (1:1), then cells were resuspended in 4CL medium 1 at a density of 150,000 cells/ml. The resuspended cells were added into flasks (Greiner Bio-One, 658190) for suspension culture. Cells formed small aggregates in 24 hours. Generally, cells were converted to ICLCs in approximately 3 weeks after initiation.

Experimental Results

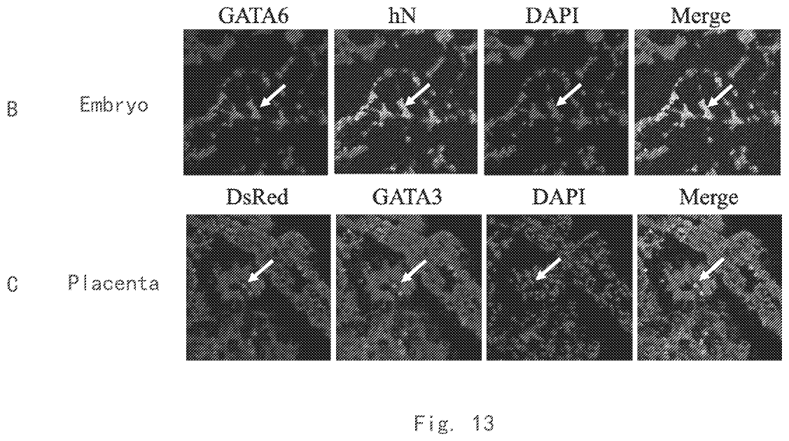

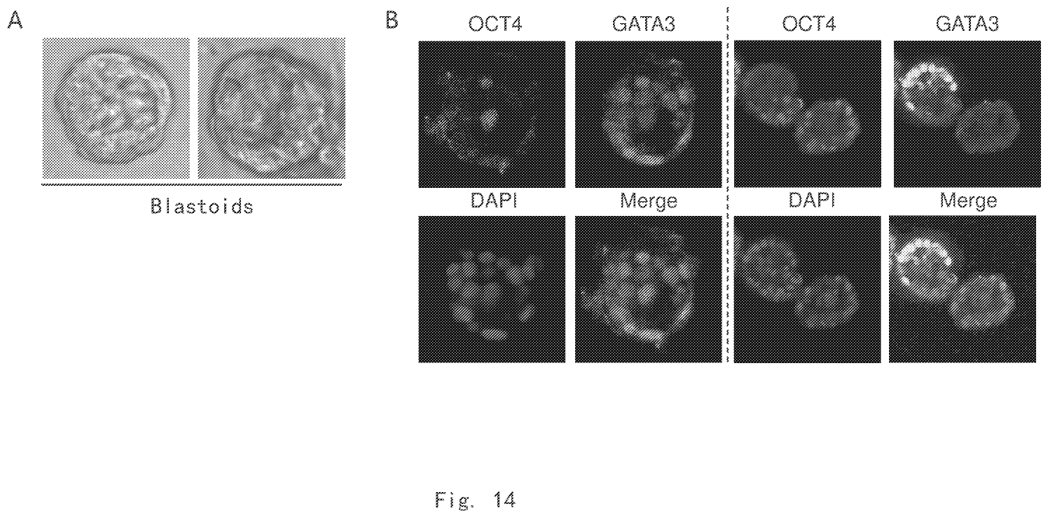

FIG. 17 is a bar chart of RT-qPCR data showing that preimplantation ICM markers KLF17, DNMT3L, DPPA5, STELLA, TFCP2L1, KLF4, MAEL, and REX1 are significantly induced in ICLCs converted in suspension using 4CL medium 1. It indicates that 4CL medium 1 is also effective for suspension culture.

### Example 5

Materials and Methods

4CL Basal Medium

Same as example 1.

4CL Supplements

4CL medium 2 (minus extracellular matrix), supplemented in the 4CL basal medium with:

SAH/PRC/EZH2 inhibitor (10 nM DZNep), HDAC inhibitor (5 nM TSA), L-ascorbic acid (50 μg/mL), JAK/STAT3 activator (20 ng/mL human LIF), MAPK/ERK inhibitor (1 μM PD0325901), tankyrase inhibitor (5 μM IWR1), ACTIVIN A/NODAL activator (20 ng/mL human ACTIVIN A), and ROCK inhibitor (1 μM Y27632).

4CL medium 3 (minus ROCK inhibitor), supplemented in the 4CL basal medium with: SAH/PRC/EZH2 inhibitor (10 nM DZNep), HDAC inhibitor (5 nM TSA), L-ascorbic acid (50 μg/mL), JAK/STAT3 activator (20 ng/mL human LIF), MAPK/ERK inhibitor (1 μM PD0325901), tankyrase inhibitor (5 μM IWR1), ACTIVIN A/NODAL activator (20 ng/mL human ACTIVIN A), extracellular matrix (0.2% (v/v) Geltrex™)

4CL medium 4 (minus ACTIVIN/NODAL activator), supplemented in the 4CL basal medium with:

SAH/PRC/EZH2 inhibitor (10 nM DZNep), HDAC inhibitor (5 nM TSA), L-ascorbic acid (50 μg/mL), JAK/STAT3 activator (20 ng/mL human LIF), MAPK/ERK inhibitor (1 μM PD0325901), tankyrase inhibitor (5 μM IWR1), extracellular matrix (0.2% (v/v) Geltrex™), and ROCK inhibitor (1 μM Y27632).

Cells

H9 human ESC line

Procedures:

Using the same procedures of example 1.

Experimental Results

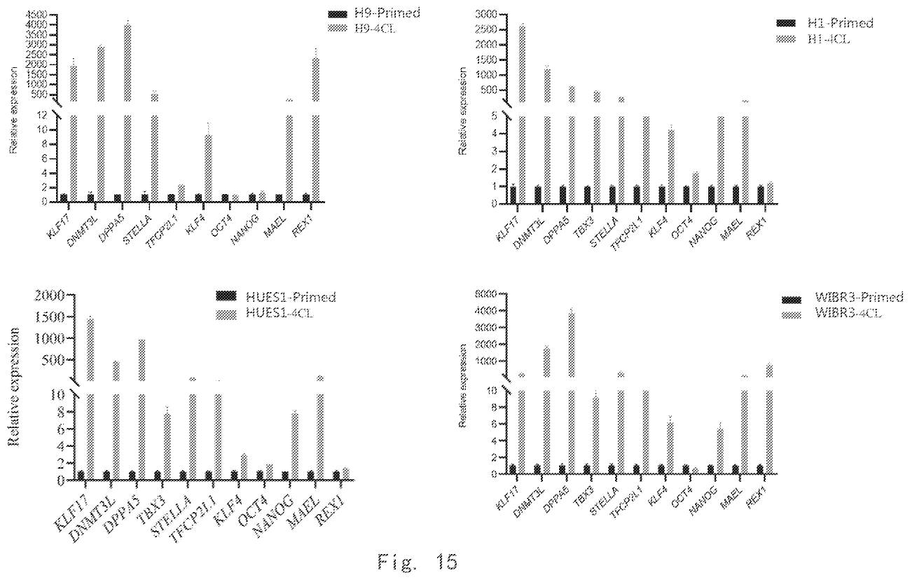

FIG. 18 shows bar charts of RT-qPCR data showing that preimplantation ICM markers KLF17, DNMT3L, DPPA5, STELLA, TFCP2L1, KLF4, MAEL, and REX1 are significantly induced in ICLCs converted using 4CL medium 2, 4CL medium 3, and 4CL medium 4, respectively. These results demonstrate that 4CL medium devoid of either Geltrex™, ROCK inhibitor, or ACTIVIN/NODAL activator is also effective.

### Example 6

Materials and Methods

4CL Basal Medium

Same as example 1.

e4CL Supplements

e4CL medium, supplemented in the 4CL basal medium with:

SAH/PRC/EZH2 inhibitor (50 nM DZNep, or 3 mM CPI-1205), HDAC inhibitor (20 nM TSA, or 1 mM VPA, or 1 mM NaB), L-ascorbic acid (50 μg/mL), JAK/STAT3 activator (20 ng/mL human LIF), MAPK/ERK inhibitor (1 μM PD0325901), tankyrase inhibitor (5 μM IWR1, or 5 μM XAV939), ACTIVIN A/NODAL activator (20 ng/mL human ACTIVIN A, or 20 ng/mL human NODAL), ROCK inhibitor (1 μM Y27632, or 1 μM thiazovivin, or 1 μM hydroxyfasudil) and extracellular matrix (0.2% (v/v) Geltrex™ or Matrigel™)

Cells

H9, H1, UH10 human ESC lines.

Procedures:

1) Conversion to 8CLCs from Primed Human PSCs

Primed human PSCs were cultured following the same procedures of example 1. One day before initiation of the conversion, primed human PSCs were dissociated into single cells and plated at 2,000 to 3,000 cells/cm 2 on feeder using mTeSR1 or E8 medium supplemented with 10 μM Y27632. Twenty-four hours later, culture medium was changed into e4CL medium, cells were cultured in incubator at 37° C., 5% CO₂, hypoxic or normoxic condition. Medium was refreshed daily. Cells were passaged every 3 to 4 days. For passaging, cells were dissociated into single cells using TrypLE: 0.5 mM EDTA (1:1), plated at 2,000 to 3,000 cells/cm² on feeder coated plates. Generally, cells were converted to 8CLCs in approximately one week.

2) Conversion to 8CLCs from ICLCs

One day before initiation of the conversion, ICLCs were dissociated into single cells and plated at 2,000 to 3,000 cells/cm 2 on feeders using 4CL medium. Twenty-four hours later, culture medium is changed into e4CL medium. Medium was refreshed daily. Cells were converted to 8CLCs in 3 to 5 days without passaging.

3) Blastoid Formation

Using the Same Procedure as Example 1.

Experimental Results

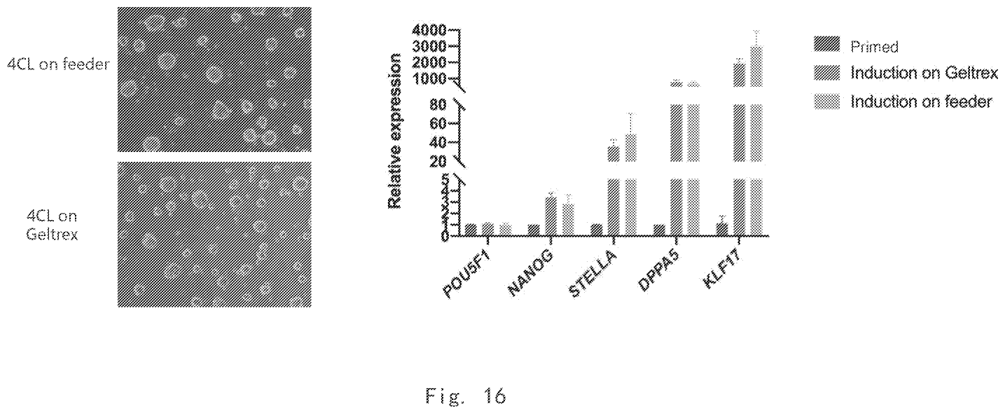

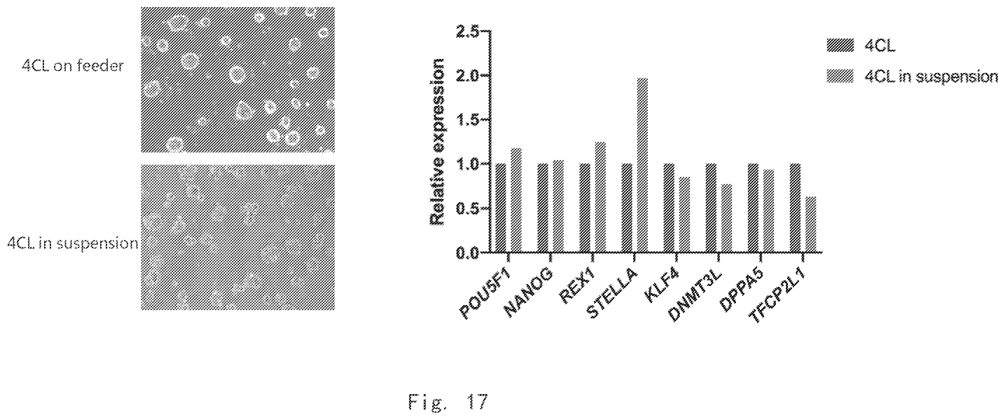

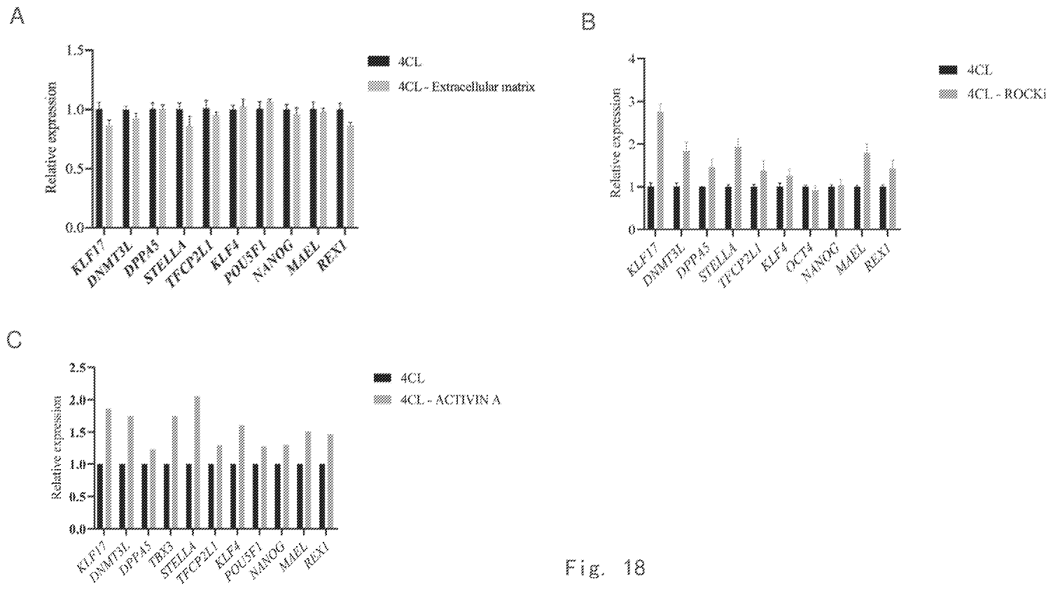

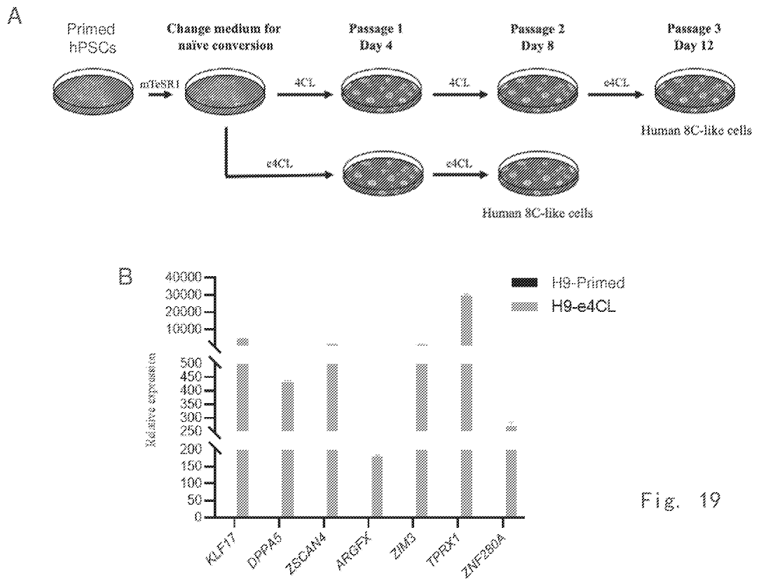

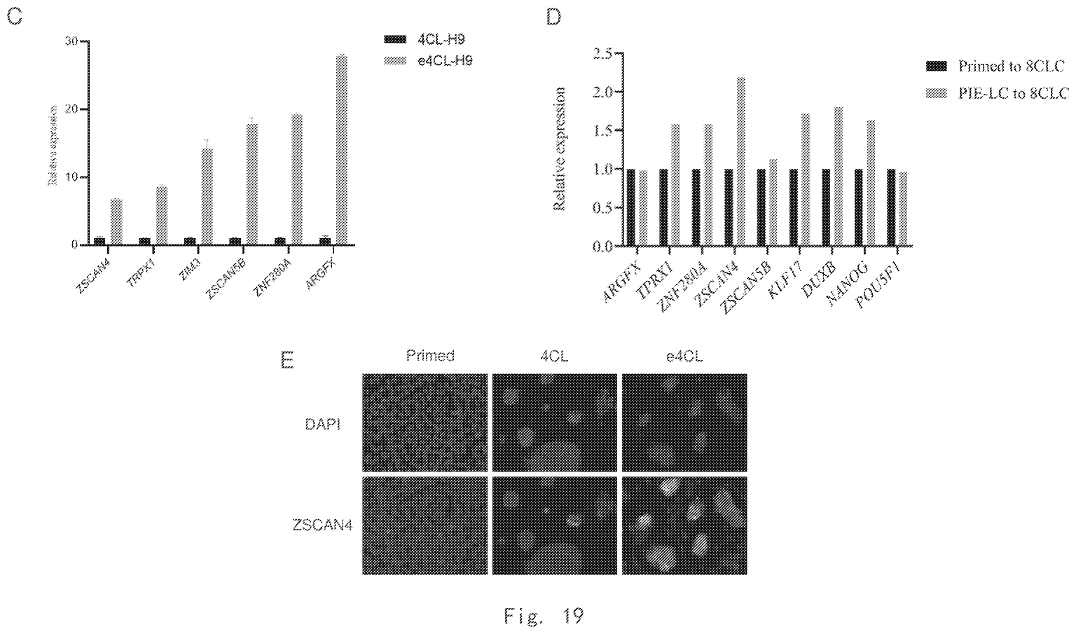

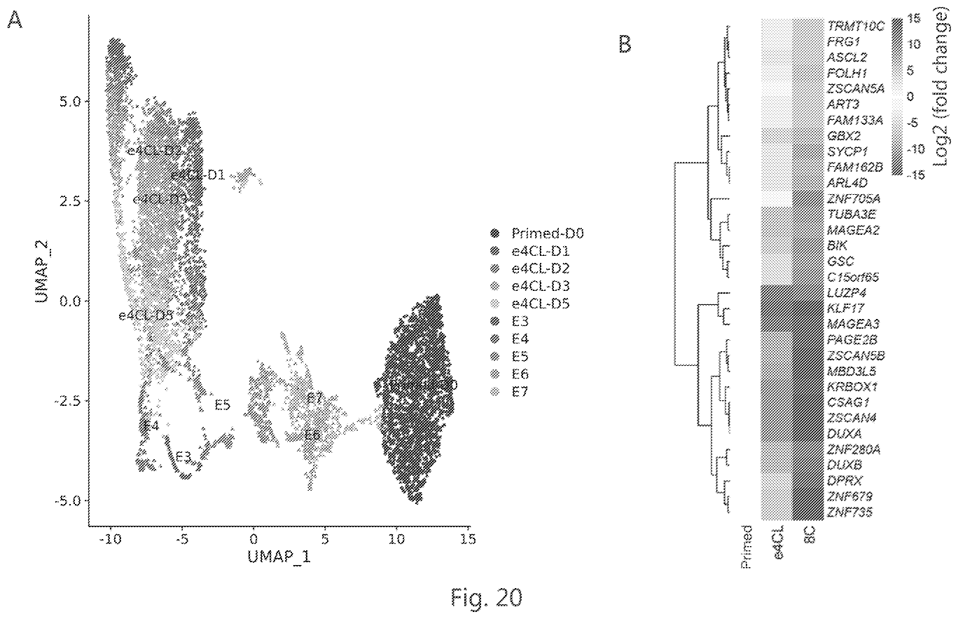

FIG. 19(A) is the scheme of 8CLCs induction procedure in two ways, one is direct induction from primed human PSCs, another is induction from ICLCs. FIG. 19(B-C) are bar charts of RT-qPCR data showing that human 8C specific markers ZSCAN4, TPRX1, ZIM3, ZSCAN5B, ZNF280A, and ARGFX are significantly induced in 8CLCs converted from primed human PSCs (FIG. 19, B) or ICLCs (FIG. 19, C). The induction level of 8C specific genes are similar in both ways of conversion (FIG. 19, D). FIG. 19(E) is immunofluorescence images showing expression of ZSCAN4 in 8CLCs. To characterize gene expression profile of 8CLCs at single cell level, the inventors performed scRNA-Seq for cells at the primed stage (primed-DO) and at days 1, 2, 3, and 5 (e4CL-D1/2/3/5) after culturing ICLCs in e4CL medium. FIG. 20(A) is a 2D scatter plot of UMAP analysis for the cells at different time points together with published scRNA-seq data of in vivo human embryos at embryonic day 3, 4, 5, 6 and 7 (E3/4/5/6/7, left panel) (from E-MTAB-3929). It illustrates that cells in e4CL medium gradually gain gene expression profiles similar to cells of human embryo at embryonic day 3 (8C stage) and day 4 (morula stage). FIG. 20(B) shows that the expression levels of human 8C stage specific markers in 8CLCs are upregulated to that of human 8C stage embryos (GSE101571). Taken together, these results indicate that 8CLCs derived by e4CL medium gain human in vivo morula and 8C stage embryo like gene expression profile.

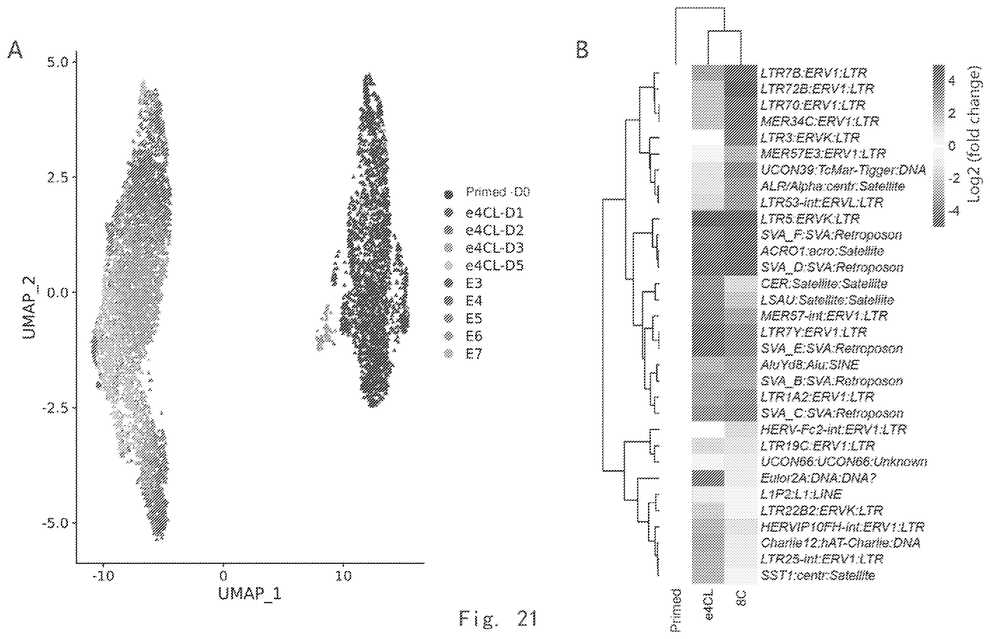

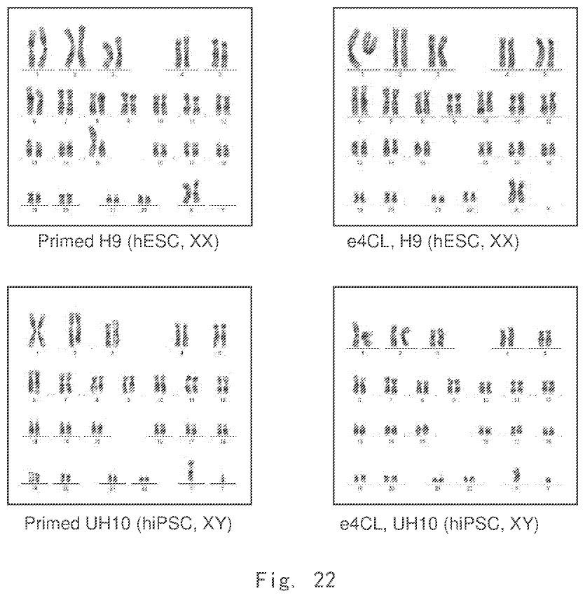

To investigate activated TEs in 8CLCs, the inventors have extracted TE profile from scRNA-seq data mentioned in FIG. 20(A). FIG. 21(A) is a 2D scatter plot of UMAP analysis for TE expression in cells at the primed stage (Primed-DO) and then at days 1, 2, 3, and 5 (D1/2/3/5) after being cultured in the e4CL medium and human embryo cells at embryonic days 3, 4, 5, 6 and 7 (E3/4/5/6/7) (from E-MTAB-3929). It depicts that cells in e4CL medium gradually gaining TE expression profiles similar to cells of human embryo at embryonic day 3 (8C stage) and day 4 (morula stage). FIG. 21(B) further illustrates that the expression levels of multiple TE subgroups in 8CLCs are induced to that of human 8C stage embryo (from GSE101571). FIG. 22 illustrates that 8CLCs maintain normal karyotype. One female human ESC line (H9) and one male human iPSC line (UH10) are shown. These results indicate that ICLCs derived by 4CL medium 1 gain human 8C stage embryo like gene expression and TE profile and maintain stable genome.

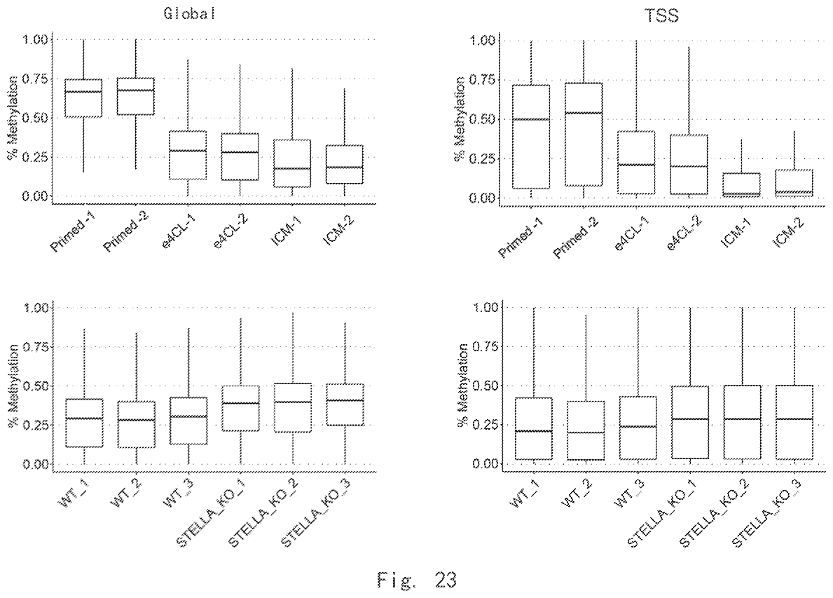

To determine the DNA methylation status of 8CLCs, the inventors have applied RRBS for 8CLCs and primed human PSCs. FIG. 23 is box plots showing the CpG DNA methylation across the whole genome (up left panel) is substantially reduced in 8CLCs compared to primed human PSCs, whereas the methylation status of TSS shows slight difference (up right panel). Of note, the reduction of DNA methylation levels in global is prevented by knocking out STELLA (down left panel). FIG. 24 shows the imprinting status of 8CLCs comparing with in vivo human embryos DNA methylation data. In addition to DNA methylation, chromatin accessibility is also changed.

Bulk ATAC-seq in FIG. 25 shows the difference of chromatin accessibility between primed human PSCs and 8CLCs. 8C specific loci which are close in primed human PSCs become open in 8CLCs, while post-implantation specific loci which are open in primed human PSCs become close. These results demonstrate that 4CL medium 1 successfully rewire epigenetic landscape towards 8C like state.

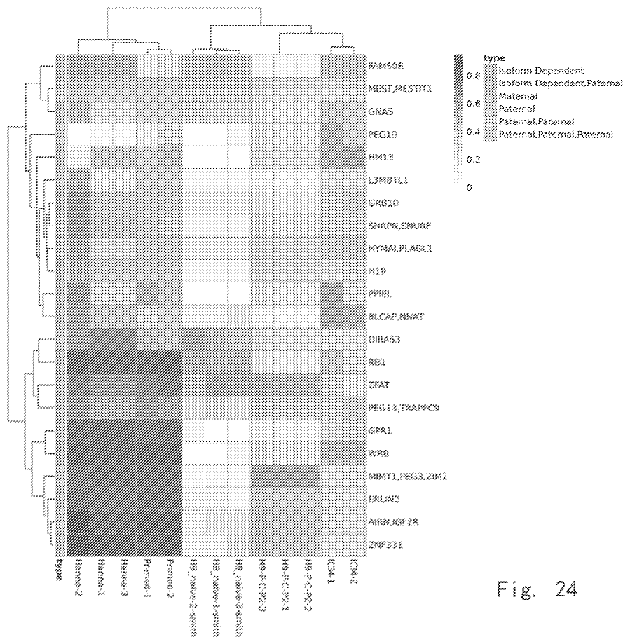

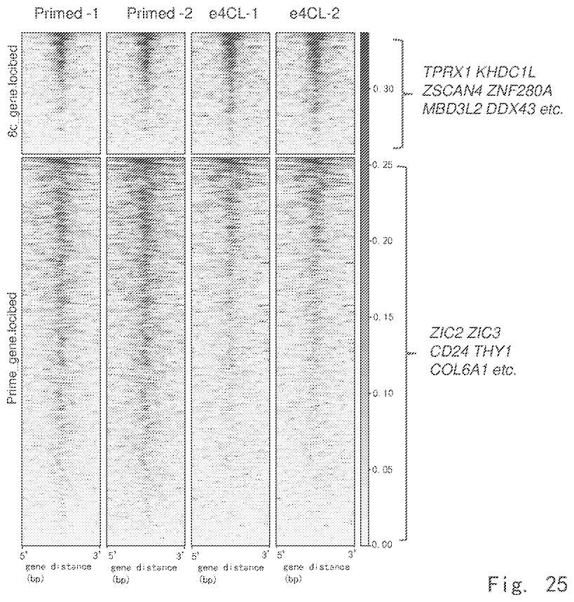

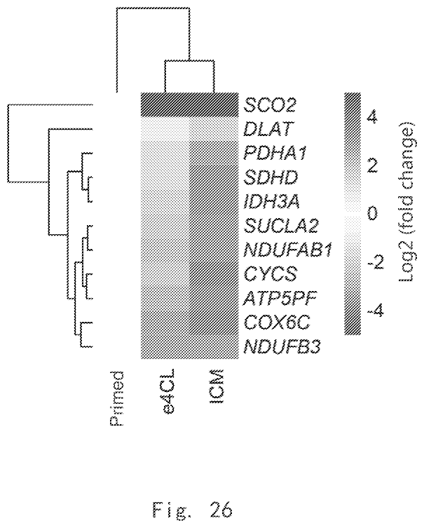

The inventors further investigated the metabolic state of 8CLCs induced in e4CL medium. Human 8C stage embryos mainly depend on oxidative phosphorylation (OxPhos)_as energy source, while predominantly depends on glycolysis after implantation. FIG. 26 shows expression level of genes related to oxidative phosphorylation is substantially upregulated in 8CLCs compared to primed human PSCs. These results imply oxidative phosphorylation is activated in 8CLCs.

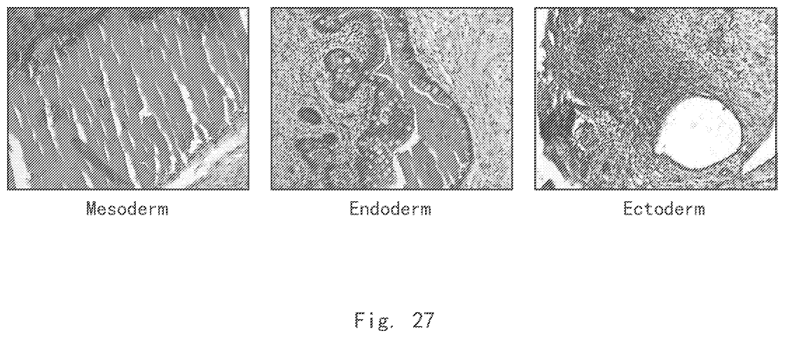

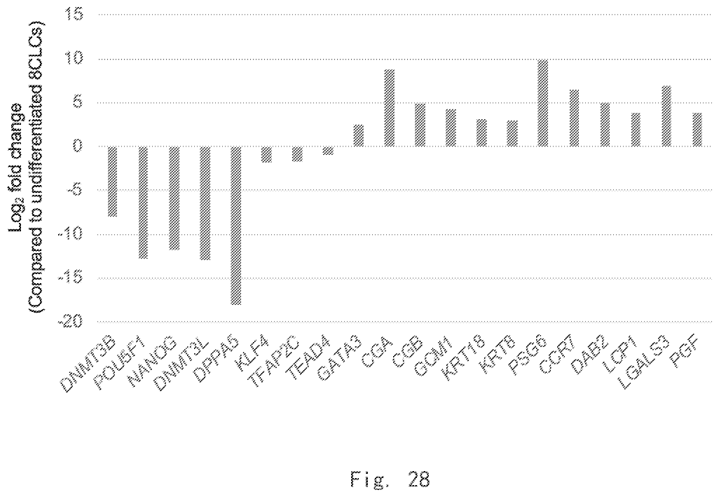

To determine the differentiation potential of 8CLCs, the inventors have performed teratoma formation assay using nude mice as receptor animal. FIG. 27 is images of hematoxylin and eosin stained teratoma tissues formed 8 weeks after injecting 1 million 8CLCs. They show the presence of all three germ layer structures: mesoderm (left panel), endoderm (middle panel) and ectoderm (right panel). The inventors also used 8CLCs to induce trophoblast stem cell-like cells (TSCLCs) using previously published protocol. As shown in FIG. 28, multiple TSC markers such as GATA3, CGA, KRT18, KRT8, PSG6, and CCR7 are significantly induced in TSCLCs compared to undifferentiated 8CLCs. These results demonstrate that 8CLCs have embryonic and extraembryonic developmental potential.

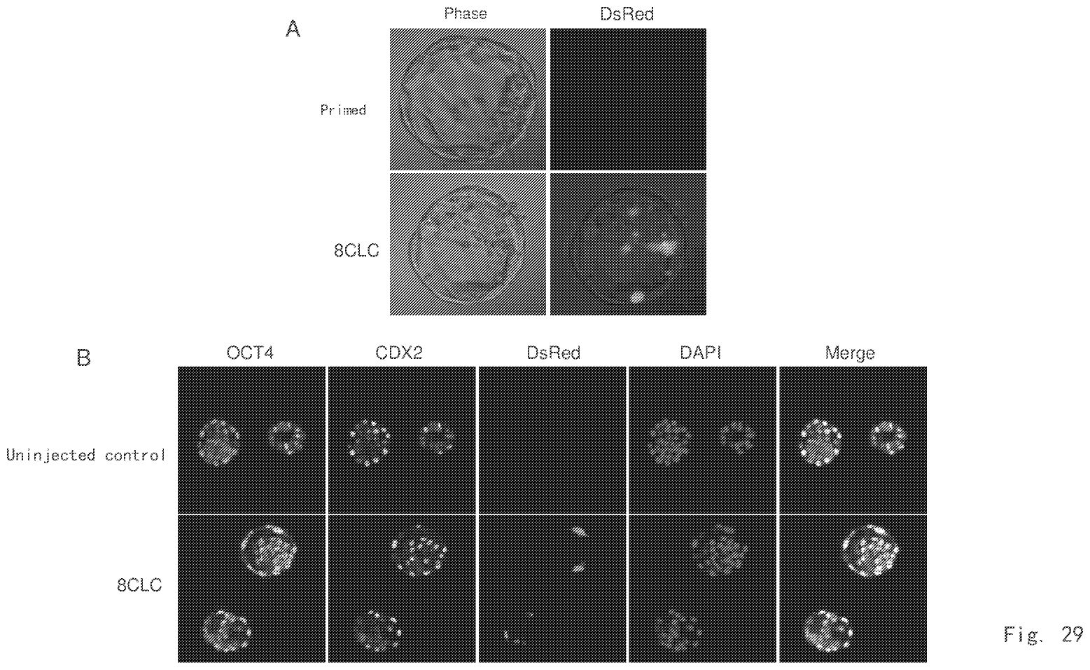

Due to ethical concerns, developmental potential cannot be tested using human embryo. Therefore, the inventors have performed cross-species chimera experiments by aggregating 8CLCs with mouse 8C stage blastomeres. Human 8CLCs are found to be successfully integrated into most mice embryos and formed chimeric blastocysts when checked after twenty-four hours of in vitro culture. At this stage, human 8CLCs are positioned in both ICM and TE parts of the chimeric blastocysts. FIG. 29(A) is representative images showing phase contrast (left) or red fluorescence channel (right) of blastocysts developed from mouse 8C blastomeres aggregated with DsRed labeled primed human PSCs (upper) or DsRed labeled 8CLCs (lower). FIG. 29(B) is immunofluorescence of chimeric blastocysts stained with anti-OCT4 (ICM, green), anti-CDX2 (TE, grey), red signal is from integrated DsRed labeled 8CLCs, and DAPI (blue) is used as nuclear counterstain. When these chimeric blastocysts were transferred into uterus of pseudo-pregnant mice and let them developed until embryonic day 10.5 (E10.5), the human cells can develop together with mice embryos and contributed into different tissues including embryonic tissues and extraembryonic placenta and yolk sac, as shown by microscope images in FIG. 30. FIG. 30(A) is representative images showing phase contrast (upper) or red fluorescence channel (lower) of E10.5 chimeric embryos (left), placenta (middle) or yolk sac (right). FIG. 30(B) is immunofluorescence images showing that hN (green) stained human cells differentiated into GATA6 (red) positive endodermal tissue. FIG. 30(C) is immunofluorescence images showing that DsRed-labeled human cells (red) differentiated into placental tissue as marked by GATA3 (green). Taken together, these results demonstrate that 8CLCs can robustly integrate into mouse blastocysts and contribute into mouse E10.5 embryonic and extraembryonic tissues in vivo.

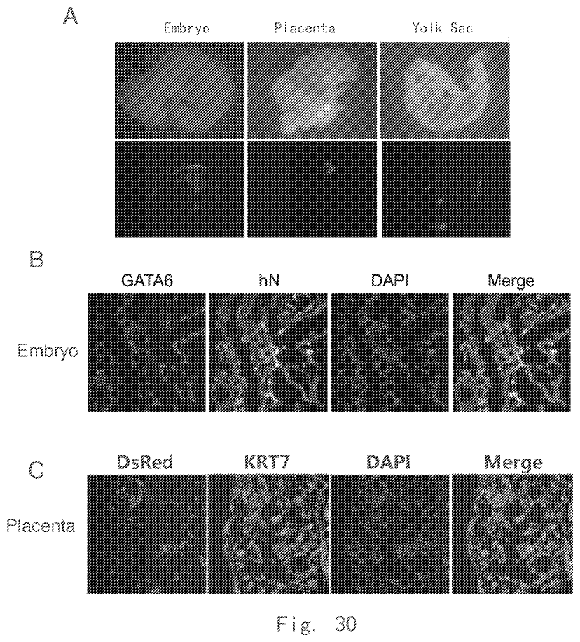

To determine the blastocyst-like structures forming potential of 8CLCs, the inventors have applied the 8CLCs to a matrix rich medium and observed blastocyst-like structures formed in 5 days, but not primed human PSCs (FIG. 31, A). FIG. 31(B) is immunofluorescence images of self-forming blastoids stained with anti-OCT4 (ICM, red), anti-GATA3 (TE, green) antibodies, or nuclear counterstain DAPI (blue).

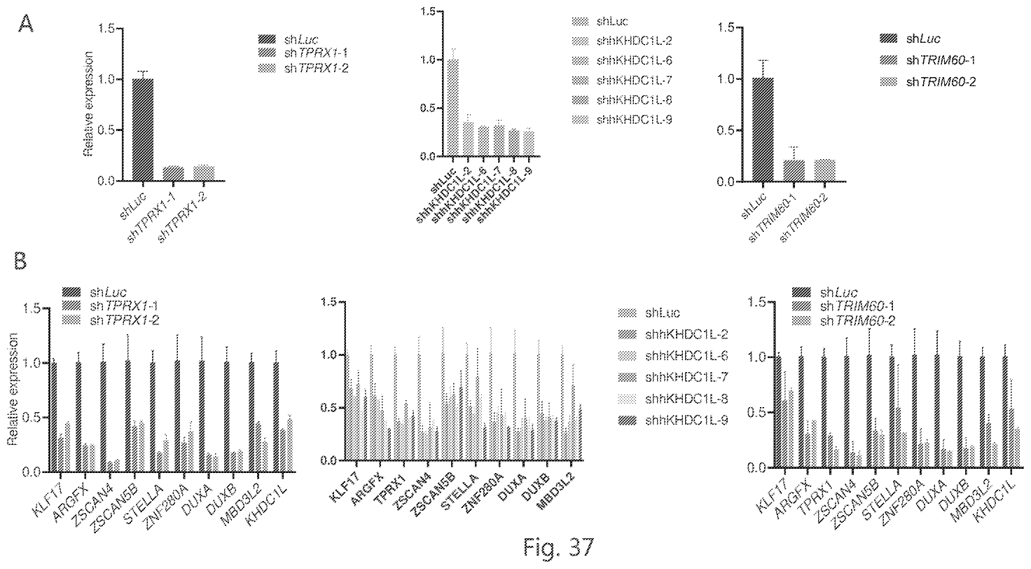

Our 8CLC can serve as a robust model for functional study of 8C regulators. In a pilot study, the inventors identified 3 potential novel regulators, TPRX1, KHDC1L, and TRIM60 that governing 8C state. FIG. 37 shows that induction of 8C specific genes during ICLC to 8CLC conversion is prohibited by TPRX1, KHDC1L, or TRIM60 knockdown.

### Example 7

Materials and Methods

e4CL Basal Medium

Same as Example 1.

e4CL Supplements

Same as Example 6.

Cells

H9 Human ESC Line.

Procedures:

Conversion to 8CLCs from ICLCs in Suspension

ICLCs were cultured following the same procedures of example 1. One day before initiation of the conversion, ICLCs were dissociated into single cells and resuspended in 4CL medium at a density of 300,000 cells/ml. The cell suspension was added into flasks for suspension culture (Greiner Bio-One, 658190). Twenty-four hours later, cells formed small aggregates and medium changed to e4CL without adding Y27632. Medium was refreshed daily, and cells were converted to 8CLCs in 3 to 5 days without passaging.

Experimental Results

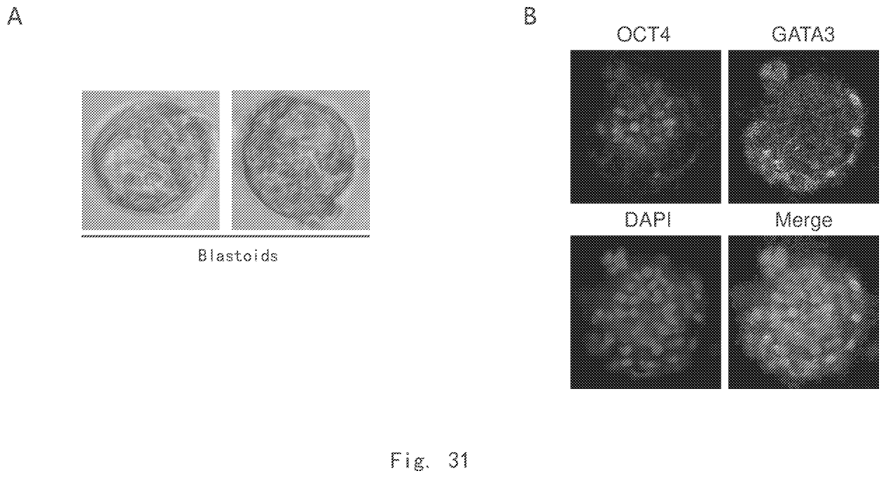

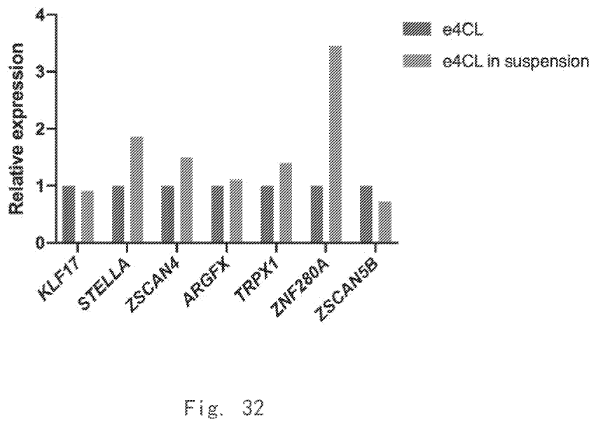

FIG. 32 is bar chart of RT-qPCR data showing 8C markers ZSCAN4, ARGFX, TPRX1, ZNF280A, and ZSCAN5B are significantly induced in 8CLCs converted in suspension using e4CL medium. It indicates that e4CL medium is also effective for suspension culture.

### Example 8

Materials and Methods

e4CL Basal Medium

Same as Example 1.

e4CL Supplements

Same as Example 6.

Cells

Human ESC Lines: HN10 and UH10

Procedures:

Same as Example 6.

Experimental Results

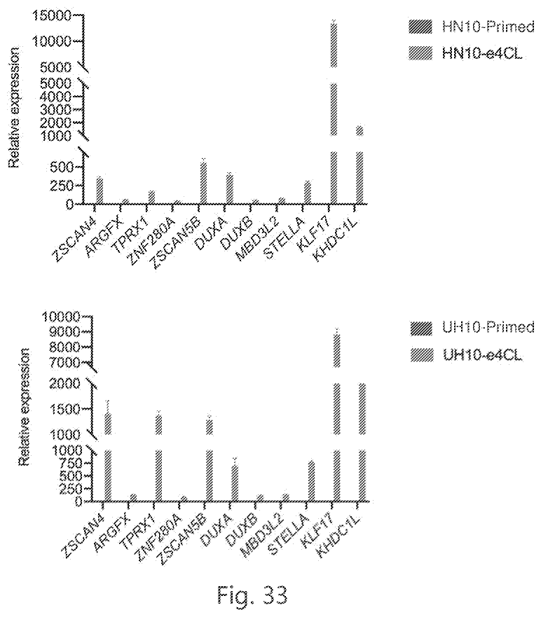

FIG. 33 is bar chart of RT-qPCR data showing 8C markers ZSCAN4, ARGFX, TPRX1, ZNF280A, ZSCAN5B, DUXA, DUXB, MBD3L2, STELLA, KLF17, and KHDC1L are significantly induced in 8CLCs converted from multiple hPSC lines. It indicates that e4CL medium is commonly applicable to human PSCs.

### Example 9

Materials and Methods

e4CL Basal Medium

Same as Example 1.

e4CL Supplements

Same as Example 6.

Cells

Mouse ESC Lines: E14 and Mervl-GFP

Procedures:

One day before initiation of the conversion, mouse ESCs cultured in serum/LIF condition were dissociated into single cells and plated on feeders using serum/LIF medium. Twenty-four hours later, culture medium is changed into e4CL medium. Medium was refreshed daily. Cells were converted to mouse2C-like state in 3 days without passaging.

Experimental Results

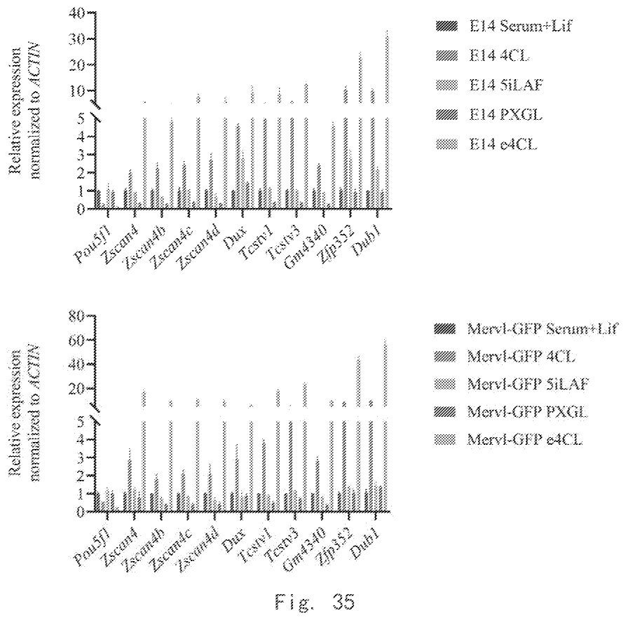

FIG. 35 shows 2C markers such as Zscan4, Zscan4b, Zscan4c, Zscan4d, Dux, Tcstv1, Tcstv3, Gm4340, Zfp352, and Dub1 are significantly induced in 2C-like cells converted from multiple mouse ESCs lines. This indicates that e4CL medium is potent to induce mouse 2C-like state as well and that is not cell line specific.

### Example 10

Materials and Methods

4CL Basal Medium

Same as Example 1.

4CL Supplements

Same as example 1, but different dosages of either PD0325901, DZNep, or TSA were also used: PD0325901, 0.5 μM; TSA, 20 nM; DZNep, 5 nM, 20 nM or 50 nM.

Cells

H9 Human ESC Line.

Procedures:

Same as Example 1.

Experimental Results

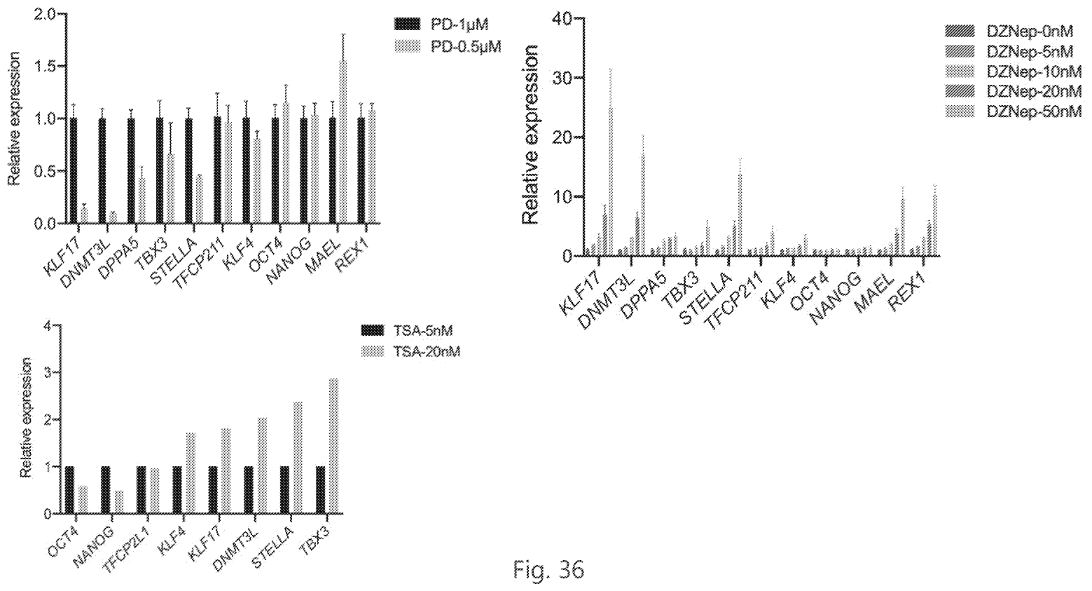

FIG. 36 depicts that preimplantation ICM markers KLF17, DNMT3L, DPPA5, STELLA, TFCP2L1, KLF4, MAEL, and REX1 are significantly induced in ICLCs converted using 4CL medium 1 supplemented with different dosage of either PD0325901, DZNep, or TSA compared to primed human PSCs cells.

### REFERENCE

Brons, I. G., Smithers, L. E., Trotter, M. W., Rugg-Gunn, P., Sun, B., Chuva de Sousa Lopes, S. M., Howlett, S. K., Clarkson, A., Ahrlund-Richter, L., Pedersen, R. A., et al. (2007). Derivation of pluripotent epiblast stem cells from mammalian embryos. Nature 448, 191-195.
Evans, M. J., and Kaufman, M. H. (1981). ESTABLISHMENT IN CULTURE OF PLURIPOTENTIAL CELLS FROM MOUSE EMBRYOS. Nature 292, 154-156.
Gafni, O., Weinberger, L., Mansour, A. A., Manor, Y. S., Chomsky, E., Ben-Yosef, D., Kalma, Y., Viukov, S., Maza, I., Zviran, A., et al. (2013). Derivation of novel human ground state naive pluripotent stem cells. Nature 504, 282-286.
Gao, X., Nowak-Imialek, M., Chen, X., Chen, D., Herrmann, D., Ruan, D., Chen, A. C. H., Eckersley-Maslin, M. A., Ahmad, S., Lee, Y. L., et al. (2019). Establishment of porcine and human expanded potential stem cells. Nat Cell Biol 21, 687-699.
Honda, A., Hatori, M., Hirose, M., Honda, C., Izu, H., Inoue, K., Hirasawa, R., Matoba, S., Togayachi, S., Miyoshi, H., et al. (2013). Naive-like conversion overcomes the limited differentiation capacity of induced pluripotent stem cells. J Biol Chem 288, 26157-26166.
Hou, P., Li, Y., Zhang, X., Liu, C., Guan, J., Li, H., Zhao, T., Ye, J., Yang, W., Liu, K., et al. (2013). Pluripotent stem cells induced from mouse somatic cells by small-molecule compounds. Science 341, 651-654.
Hu, K. (2019). On Mammalian Totipotency: What Is the Molecular Underpinning for the Totipotency of Zygote? Stem Cells Dev 28, 897-906.
Li, R., Zhong, C., Yu, Y., Liu, H., Sakurai, M., Yu, L., Min, Z., Shi, L., Wei, Y., Takahashi, Y., et al. (2019). Generation of Blastocyst-like Structures from Mouse Embryonic and Adult Cell Cultures. Cell 179, 687-702 e618.
Li, Y., Zhang, Z., Chen, J., Liu, W., Lai, W., Liu, B., Li, X., Liu, L., Xu, S., Dong, Q., et al. (2018). Stella safeguards the oocyte methylome by preventing de novo methylation mediated by DNMT1. Nature 564, 136-140.
Macfarlan, T. S., Gifford, W. D., Driscoll, S., Lettieri, K., Rowe, H. M., Bonanomi, D., Firth, A., Singer, O., Trono, D., and Pfaff, S. L. (2012). Embryonic stem cell potency fluctuates with endogenous retrovirus activity. Nature 487, 57-63.
Nakamura, T., Liu, Y. J., Nakashima, H., Umehara, H., Inoue, K., Matoba, S., Tachibana, M., Ogura, A., Shinkai, Y., and Nakano, T. (2012). PGC7 binds histone H3K9me2 to protect against conversion of 5 mC to 5 hmC in early embryos. Nature 486, 415-419.
Nichols, J., and Smith, A. (2011). The origin and identity of embryonic stem cells. Development 138, 3-8.
Shahbazi, M. N., and Zernicka-Goetz, M. (2018). Deconstructing and reconstructing the mouse and human early embryo. Nat Cell Biol 20, 878-887.
Shi, Y., Inoue, H., Wu, J. C., and Yamanaka, S. (2017). Induced pluripotent stem cell technology: a decade of progress. Nat Rev Drug Discov 16, 115-130.
Takahashi, K., and Yamanaka, S. (2006). Induction of pluripotent stem cells from mouse embryonic and adult fibroblast cultures by defined factors. Cell 126, 663-676.
Takashima, Y., Guo, G., Loos, R., Nichols, J., Ficz, G., Krueger, F., Oxley, D., Santos, F., Clarke, J., Mansfield, W., et al. (2014). Resetting transcription factor control circuitry toward ground-state pluripotency in human. Cell 158, 1254-1269.
Tesar, P. J., Chenoweth, J. G., Brook, F. A., Davies, T. J., Evans, E. P., Mack, D. L., Gardner, R. L., and McKay, R. D. (2007). New cell lines from mouse epiblast share defining features with human embryonic stem cells. Nature 448, 196-199.
Theunissen, T. W., Powell, B. E., Wang, H., Mitalipova, M., Faddah, D. A., Reddy, J., Fan, Z. P., Maetzel, D., Ganz, K., Shi, L., et al. (2014). Systematic Identification of Culture Conditions for Induction and Maintenance of Naive Human Pluripotency. Cell Stem Cell 15, 524-526.
Thomson, J. A., Itskovitz-Eldor, J., Shapiro, S. S., Waknitz, M. A., Swiergiel, J. J., Marshall, V. S., and Jones, J. M. (1998). Embryonic stem cell lines derived from human blastocysts. Science 282, 1145-1147.
Yang, Y., Liu, B., Xu, J., Wang, J., Wu, J., Shi, C., Xu, Y., Dong, J., Wang, C., Lai, W., et al. (2017). Derivation of Pluripotent Stem Cells with In Vivo Embryonic and Extraembryonic Potency. Cell 169, 243-257 e225.
Ying, Q. L., Wray, J., Nichols, J., Batlle-Morera, L., Doble, B., Woodgett, J., Cohen, P., and Smith, A. (2008). The ground state of embryonic stem cell self-renewal. Nature 453, 519-523.
Zhu, P., Guo, H., Ren, Y., Hou, Y., Dong, J., Li, R., Lian, Y., Fan, X., Hu, B., Gao, Y., et al. (2018). Single-cell DNA methylome sequencing of human preimplantation embryos. Nat Genet 50, 12-19.

1. A chemically defined culture medium for culturing PSCs comprising a basal medium for culturing stem cells supplemented with a HDAC inhibitor and one or more of a PRC and EZH2 inhibitor.

2. The chemically defined culture medium according to claim 1, wherein the PRC or EZH2 inhibitor is a SAH inhibitor.

3. The chemically defined culture medium according to claim 1, wherein the culture medium is further supplemented with one or more components selected from a group consisting of L-ascorbic acid or a derivative thereof, an activator of JAK/STAT3 signaling, an inhibitor of MAPK/ERK signaling and a tankyrase inhibitor; optionally, the culture medium is further supplemented with one or more components selected from a group consisting of an activator of ACTIVIN/NODAL signaling, a ROCK inhibitor, and an extracellular matrix.

4. The chemically defined culture medium according to claim 2, wherein:
the PRC/EZH2 inhibitor or the SAH inhibitor is selected from a group consisting of DZNep and CPI-1205; preferably, the final concentration of DZNep in the culture medium is from 5 to 80 nM, preferably 5 to 50 nM; preferably, the final concentration of CPI-1205 in the culture medium is from 0.5 to 5 mM, preferably 1 to 3 mM;
the HDAC inhibitor is selected from a group consisting of TSA, VPA and NaB; preferably, the final concentration of TSA in the culture medium is from 3 to 30 nM, preferably 3 to 25 nM; preferably, the final concentration of VPA in the culture medium is from 0.25 to 2 mM, preferably 0.5 to 1.5 mM; preferably, the final concentration of NaB in the culture medium is from 0.25 to 2 mM, preferably 0.5 to 1.5 mM;
the final concentration of L-ascorbic acid in the culture medium is 40 to 70 μg/mL;
the final concentration of the activator of JAK/STAT3 signaling in the culture medium is 10 to 50 ng/mL; preferably, the activator of JAK/STAT3 signaling is LIF;
the final concentration of the inhibitor of MAPK/ERK signaling in the culture medium is 0.5 μM to 3 μM; preferably, the inhibitor of MAPK/ERK signaling is PD0325901;
the final concentration of the tankyrase inhibitor in the culture medium is from 2 to 8 μM; preferably, the tankyrase inhibitor is selected from a group consisting of IWR1 and XAV939;
the final concentration of the activator of ACTIVIN/NODAL signaling is from 10 to 25 ng/mL; preferably, the activator of ACTIVIN/NODAL signaling is selected from a group consisting of ACTIVIN A and NODAL;
the final concentration of the ROCK inhibitor in the culture medium is from 0.5 to 2 μM; preferably, the ROCK inhibitor is selected from a group consisting of Y27632, thiazovivin and hydroxyfasudil; and
the amount of the extracellular matrix in the culture medium is 0.1 to 0.5% (v/v); preferably, the extracellular matrix is selected from a group consisting of Matrigel™, Geltrex™ and ECM™.

5. (canceled)

6. The chemically defined culture medium according to claim 1, wherein the culture medium comprises:
(A) DZNep at a final concentration of 5 to 15 nM or CPI-1205 at a final concentration of to 2 mM, and TSA at a final concentration of 3 to 30 nM, or VPA at a final concentration of to 2 mM, or NaB at a final concentration of 0.25 to 2 mM, preferably TSA at a final concentration of 3 to 10 nM, or VPA at a final concentration of 0.25 to 1 mM, or NaB at a final concentration of 0.25 to 1 mM; or DZNep at a final concentration of 5 to 80 nM, preferably 5 to nM or CPI-1205 at a final concentration of 0.5 to 5 mM, preferably 0.5 to 3 mM, and TSA at a final concentration of 3 to 10 nM, or VPA at a final concentration of 0.25 to 0.5 mM, or NaB at a final concentration of 0.25 to 0.5 mM;
(B) L-ascorbic acid at a final concentration of 40 to 70 μg/mL;
(C) LIF at a final concentration of 10 to 30 ng/mL;
(D) PD0325901 at a final concentration of 0.5 to 1.5 μM;
(E) IWR1 or XAV939 at a final concentration of 3 to 6 μM; and the culture medium is further supplemented with:
(1) ACTIVIN A or NODAL at a final concentration of 10 to 25 ng/mL; Y27632, thiazovivin or hydroxyfasudil at a final concentration in a range 0.5 to 2 μM; and an extracellular matrix in an amount of 0.1% to 0.5% (v/v); or
(2) ACTIVIN A or NODAL at a final concentration of 10 to 25 ng/mL; and Y27632, thiazovivin or hydroxyfasudil at a final concentration in a range 0.5 to 2 μM; or
(3) ACTIVIN A or NODAL at a final concentration of 10 to 25 ng/mL; and an extracellular matrix in an amount of 0.1% to 0.5% (v/v); or
(4) Y27632, thiazovivin or hydroxyfasudil at a final concentration of 0.5 to 2 μM; and an extracellular matrix in an amount of 0.1% to 0.5% (v/v); or
(5) ACTIVIN A or NODAL at a final concentration of 10 to 25 ng/mL; or Y27632, thiazovivin or hydroxyfasudil at a final concentration of 0.5 to 2 μM; or an extracellular matrix in an amount of 0.1% to 0.5% (v/v),
preferably, the culture medium comprises 10 nM DZNep or 1 mM CPI-1205; 5 nM TSA, or 0.5 mM VPA, or 0.5 mM NaB; 50 μg/mL L-ascorbic acid; 20 ng/mL LIF; 1 μM PD0325901; and 5 μM IWR1 or 5 μM XAV939; and is further supplemented with (1) 20 ng/mL of ACTIVIN A or NODAL, 1 μM of Y27632, thiazovivin or hydroxyfasudil, and 0.2% (v/v) of an extracellular matrix; or (2) 20 ng/mL of ACTIVIN A or NODAL, and 1 μM of Y27632, thiazovivin or hydroxyfasudil; (3) 20 ng/mL of ACTIVIN A or NODAL, and 0.2% (v/v) of an extracellular matrix; or (4) 1 μM of Y27632, thiazovivin or hydroxyfasudil, and 0.2% (v/v) of an extracellular matrix; or (5) 20 ng/mL of ACTIVIN A or NODAL, or 1 μM of Y27632, thiazovivin or hydroxyfasudil, or 0.2% (v/v) of an extracellular matrix.

7. (canceled)

8. The chemically defined culture medium according to claim 1, wherein the culture medium comprises DZNep at a final concentration of 40 to 70 nM or CPI-1205 at a final concentration of 2 to 4 mM; TSA at a final concentration of 10 to 30 nM, or VPA at a final concentration of 0.5 to 1.5 mM or NaB at a final concentration of 0.5 to 1.5 mM; L-ascorbic acid at a final concentration of 40 to 70 μg/mL; LIF at a final concentration of 10 to 30 ng/mL; PD0325901 at a final concentration of 0.5 to 1.5 μM; and IWR1 or XAV939 each at a final concentration of 3 to 6 μM; and is further supplemented with:
(1) ACTIVIN A or NODAL at a final concentration of 10 to 25 ng/mL; Y27632, thiazovivin or hydroxyfasudil at a final concentration in a range 0.5 to 2 μM; and an extracellular matrix in an amount of 0.1% to 0.5% (v/v); or
(2) ACTIVIN A or NODAL at a final concentration of 10 to 25 ng/mL; and Y27632, thiazovivin or hydroxyfasudil at a final concentration of 0.5 to 2 μM; or
(3) ACTIVIN A or NODAL at a final concentration of 10 to 25 ng/mL; and an extracellular matrix in an amount of 0.1% to 0.5% (v/v); or
(4) Y27632, thiazovivin or hydroxyfasudil at a final concentration of 0.5 to 2 μM; and an extracellular matrix in an amount of 0.1% to 0.5% (v/v); or
(5) ACTIVIN A or NODAL at a final concentration of 10 to 25 ng/mL; or Y27632, thiazovivin or hydroxyfasudil at a final concentration in a range 0.5 to 2 μM; or an extracellular matrix in an amount of 0.1% to 0.5% (v/v),
preferably, the culture medium comprises 50 nM DZNep or 3 mM CPI-1205; 20 nM TSA, or 1 mM VPA, or 1 mM NaB; 50 μg/mL L-ascorbic acid; 20 ng/mL LIF; 1 μM PD0325901; and 5 μM IWR1 or 5 μM XAV939; and is further supplemented with (1) 20 ng/mL of ACTIVIN A or NODAL, 1 μM of Y27632, thiazovivin or hydroxyfasudil, and 0.2% (v/v) of an extracellular matrix; or (2) 20 ng/mL of ACTIVIN A or NODAL, and 1 μM of Y27632, thiazovivin or hydroxyfasudil; (3) 20 ng/mL of ACTIVIN A or NODAL, and 0.2% (v/v) of an extracellular matrix; or (4) 1 μM of Y27632, thiazovivin or hydroxyfasudil, and 0.2% (v/v) of an extracellular matrix; or (5) 20 ng/mL of ACTIVIN A or NODAL, or 1 μM of Y27632, thiazovivin or hydroxyfasudil, or 0.2% (v/v) of an extracellular matrix.

9. (canceled)

10. The chemically defined culture medium according to claim 1, wherein the basal medium is selected from a group consisting of Dulbecco's modified eagle's medium (DMEM), minimal essential medium (MEM), basal medium Eagle (BME), RPMI1640, F10, F12, α minimal essential medium (a MEM), Glasgow's minimal essential medium (GMEM), Iscove's modified Dulbecco's medium, Neurobasal Medium, DMEM/F12 and Advanced DMEM/F12 and a combination thereof; preferably, the basal medium is a mixture of Advanced DMEM/F12 and Neurobasal Medium in a ratio of 1:1 (v/v).

11. The chemically defined culture medium according to claim 1, wherein the culture medium is further supplemented with one or more components selected from a group consisting of serum replacement, alternative carbon source, non-essential amino acid, L-glutamine or its alternative and antibiotic.

12. The chemically defined culture medium according to claim 11, wherein:
the serum replacement is selected from a group consisting of KOSR, N2 and B27, and combinations thereof; preferably, the serum replacement is a mixture of N2 and B27 in a ratio of 1:1 (w/w);
the alternative carbon source is pyruvate, such as sodium pyruvate;
the L-glutamine or its alternative is Glutamax™ supplement comprising L-alanyl-L-glutamine dipeptide in 0.85% NaCl; and/or
the antibiotic is selected from a group consisting of penicillin, streptomycin, or a mixture of penicillin and streptomycin.

13. A method for converting primate PSCs to one or more of ICLCs and 8CLCs or for converting ICLCs to 8CLCs, comprising culturing the primate PSCs or ICLCs in the presence of a PRC/EZH2 inhibitor and a HDAC inhibitor; preferably, the PRC/EZH2 inhibitor is a SAH inhibitor.

14. The method according to claim 13, wherein the method comprises culturing primate PSCs or ICLCs in the presence of the PRC/EZH2 inhibitor and the HDAC inhibitor, and one or more components selected from a group consisting of L-ascorbic acid, an activator of JAK/STAT3 signaling, an inhibitor of MAPK/ERK signaling and a tankyrase inhibitor, and optionally in the presence of one or more components selected from a group consisting of an activator of ACTIVIN/NODAL signaling, a ROCK inhibitor, and an extracellular matrix, wherein
the PRC/EZH2 inhibitor or the SAH inhibitor is selected from a group consisting of DZNep and CPI-1205;
the HDAC inhibitor is selected from a group consisting of TSA, VPA and NaB;
preferably, primate PSCs or ICLCs are cultured in the presence of DZNep at a final concentration of 5 to 80 nM, preferably 5 to 50 nM or CPI-1205 at a final concentration of 0.5 to mM, preferably 1.5 to 3 mM, and in the presence of TSA at a final concentration of 3 to 30 nM, preferably 3 to 25 nM, or VPA at a final concentration of 0.25 to 2 mM, preferably 0.5 to 1.5 mM, or NaB at a final concentration of 0.25 to 2 mM, preferably 0.5 to 1.5 mM.

15. (canceled)

16. The method according to claim 14, wherein:
L-ascorbic acid is present at a final concentration of 40 to 70 μg/mL;
the final concentration of the activator of JAK/STAT3 signaling is 10 to 50 ng/mL; preferably the activator of JAK/STAT3 signaling is LIF;
the final concentration of the inhibitor of MAPK/ERK signaling is 0.5 to 3 μM; preferably, the inhibitor of MAPK/ERK signaling is PD0325901;
the final concentration of the tankyrase inhibitor is 2 to 8 μM; preferably, the tankyrase inhibitor is selected from a group consisting of IWR1 and XAV939;
the final concentration of the activator of ACTIVIN/NODAL signaling is from 10 to 25 ng/mL; preferably, the activator of ACTIVIN/NODAL signaling is selected from a group consisting of ACTIVIN A and NODAL;
the final concentration of the ROCK inhibitor is 0.5 to 2 μM; preferably, the ROCK inhibitor is selected from a group consisting of Y27632, thiazovivin, and hydroxyfasudil; and
the extracellular matrix is present at an amount of 0.1% to 0.5% (v/v); preferably, the extracellular matrix is selected from a group consisting of Matrigel™, Geltrex™ and ECM™.

17-18. (canceled)

19. The method according to claim 13, wherein converting primate PSCs to ICLCs, further comprises:
(a) genetically engineering the primate PSCs to reduce the activity of one or more of SAH, PRC and EZH2 of the PSCs by knockdown or knockout of one or more relevant genes in the cells; and
(b) culturing the genetically engineered cells obtained in step (a) in a culture medium comprising: TSA at a final concentration of 3 to 30 nM, or VPA at a final concentration of 0.25 to 2 mM, or NaB at a final concentration of 0.25 to 2 mM, preferably TSA at a final concentration of 3 to 10 nM, or VPA at a final concentration of 0.25 to 1 mM, or NaB at a final concentration of 0.25 to 1 mM, and optionally DZNep at a final concentration of 5 to 15 nM or CPI-1205 at a final concentration of 0.5 to 2 mM, or TSA at a final concentration of 3 to 10 nM, or VPA at a final concentration of 0.25 to 0.5 mM, or NaB at a final concentration of 0.25 to 0.5 mM and optionally DZNep at a final concentration of 5 to 80 nM, preferably 5 to 50 nM or CPI-1205 at a final concentration of 0.5 to 5 mM; L-ascorbic acid at a final concentration of 40 to 70 μg/mL; LIF at a final concentration of 10 to 30 ng/mL; PD0325901 at a final concentration of 0.5 to 1.5 μM; IWR1 or XAV939 at a final concentration of 3 to 6 μM; wherein the culture medium is further supplemented with:
(1) ACTIVIN A or NODAL at a final concentration of 10 to 25 ng/mL; Y27632, thiazovivin or hydroxyfasudil at a final concentration in a range 0.5 to 2 μM; and an extracellular matrix in an amount of 0.1% to 0.5% (v/v); or
(2) ACTIVIN A or NODAL at a final concentration of 10 to 25 ng/mL; and Y27632, thiazovivin or hydroxyfasudil at a final concentration in a range 0.5 to 2 μM; or
(3) ACTIVIN A or NODAL at a final concentration of 10 to 25 ng/mL; and an extracellular matrix in an amount of 0.1% to 0.5% (v/v); or
(4) Y27632, thiazovivin or hydroxyfasudil at a final concentration of 0.5 to 2 μM; and an extracellular matrix in an amount of 0.1% to 0.5% (v/v); or
(5) ACTIVIN A or NODAL at a final concentration of 10 to 25 ng/mL; or Y27632, thiazovivin or hydroxyfasudil at a final concentration of 0.5 to 2 μM; or an extracellular matrix in an amount of 0.1% to 0.5% (v/v);
preferably the culture medium comprises: 5 nM TSA, or 0.5 mM VPA, or 0.5 mM NaB; 50 μg/mL L-ascorbic acid; 20 ng/mL LIF; 1 μM PD0325901; 5 μM IWR1 or 5 μM XAV939; and optionally 10 nM DZNep or 1 mM CPI-1205; and wherein the culture medium is further supplemented with (1) 20 ng/mL of ACTIVIN A or NODAL, 1 μM of Y27632, thiazovivin or hydroxyfasudil, and 0.2% (v/v) of an extracellular matrix; or (2) 20 ng/mL of ACTIVIN A or NODAL, and 1 μM of Y27632, thiazovivin or hydroxyfasudil; (3) 20 ng/mL of ACTIVIN A or NODAL, and 0.2% (v/v) of an extracellular matrix; or (4) 1 μM of Y27632, thiazovivin or hydroxyfasudil, and 0.2% (v/v) of an extracellular matrix; or (5) 20 ng/mL of ACTIVIN A or NODAL, or 1 μM of Y27632, thiazovivin or hydroxyfasudil, or 0.2% (v/v) of an extracellular matrix,
wherein the basal medium of the culture medium is selected from a group consisting of Dulbecco's modified eagle's medium (DMEM), minimal essential medium (MEM), basal medium Eagle (BME), RPMI1640, F10, F12, a minimal essential medium (a MEM), Glasgow's minimal essential medium (GMEM), Iscove's modified Dulbecco's medium, Neurobasal Medium and DMEM/F12, and a combination thereof; preferably, the basal medium is a mixture of Advanced DMEM/F12 and Neurobasal Medium in a ratio of 1:1 (v/v).

20. The method according to claim 13, wherein converting primate PSCs or ICLCs to 8CLCs, further comprises:
(a) genetically engineering the primate PSCs or ICLCs to reduce the activity of any one of SAH, PRC and EZH2 of the PSCs or ICLCs by knockdown or knockout of one or more relevant genes in the cells;
(b) culturing the genetically engineered cells obtained in step (a) in a culture medium comprising: TSA at a final concentration of 10 to 30 nM, or VPA at a final concentration of 0.5 to 1.5 mM or NaB at a final concentration of 0.5 to 1.5 mM; L-ascorbic acid at a final concentration of 40 to 70 μg/mL; LIF at a final concentration of 10 to 30 ng/mL; PD0325901 at a final concentration of 0.5 to 1.5 μM; IWR1 or XAV939 each at a final concentration of 3 to 6 μM; and optionally DZNep at a final concentration of 40 to 70 nM or CPI-1205 at a final concentration of 2 to 4 mM; and wherein the culture medium is further supplemented with:
(1) ACTIVIN A or NODAL at a final concentration of 10 to 25 ng/mL; Y27632, thiazovivin or hydroxyfasudil at a final concentration in a range 0.5 to 2 μM; and an extracellular matrix in an amount of 0.1% to 0.5% (v/v); or
(2) ACTIVIN A or NODAL at a final concentration of 10 to 25 ng/mL; and Y27632, thiazovivin or hydroxyfasudil at a final concentration of 0.5 to 2 μM; or
(3) ACTIVIN A or NODAL at a final concentration of 10 to 25 ng/mL; and an extracellular matrix in an amount of 0.1% to 0.5% (v/v); or
(4) Y27632, thiazovivin or hydroxyfasudil at a final concentration of 0.5 to 2 μM; and an extracellular matrix in an amount of 0.1% to 0.5% (v/v); or
(5) ACTIVIN A or NODAL at a final concentration of 10 to 25 ng/mL; or Y27632, thiazovivin or hydroxyfasudil at a final concentration in a range 0.5 to 2 μM; or an extracellular matrix in an amount of 0.1% to 0.5% (v/v);
preferably, the culture medium comprises: 20 nM TSA, or 1 mM VPA, or 1 mM NaB; 50 μg/mL L-ascorbic acid; 20 ng/mL LIF; 1 μM PD0325901; 5 μM IWR1 or 5 μM XAV939; and optionally 50 nM DZNep or 3 mM CPI-1205; and wherein the culture medium is further supplemented with (1) 20 ng/mL of ACTIVIN A or NODAL, 1 μM of Y27632, thiazovivin or hydroxyfasudil, and 0.2% (v/v) of an extracellular matrix; or (2) 20 ng/mL of ACTIVIN A or NODAL, and 1 μM of Y27632, thiazovivin or hydroxyfasudil; (3) 20 ng/mL of ACTIVIN A or NODAL, and 0.2% (v/v) of an extracellular matrix; or (4) 1 μM of Y27632, thiazovivin or hydroxyfasudil, and 0.2% (v/v) of an extracellular matrix; or (5) 20 ng/mL of ACTIVIN A or NODAL, or 1 μM of Y27632, thiazovivin or hydroxyfasudil, or 0.2% (v/v) of an extracellular matrix,
wherein the basal medium of the culture medium is selected from a group consisting of Dulbecco's modified eagle's medium (DMEM), minimal essential medium (MEM), basal medium Eagle (BME), RPMI1640, F10, F12, a minimal essential medium (a MEM), Glasgow's minimal essential medium (GMEM), Iscove's modified Dulbecco's medium, Neurobasal Medium, DMEM/F12 and Advanced DMEM/F12, and a combination thereof; preferably, the basal medium is a mixture of Advanced DMEM/F12 and Neurobasal Medium in a ratio of 1:1 (v/v).

21. The method according to claim 13, wherein converting primate PSCs to ICLCs comprises:
(a) genetically engineering the primate PSCs to reduce the activity of HDAC of the PSCs by knockdown and/or knockout of one or more relevant genes in the cells;
(b) culturing the genetically engineered cells obtained in step (a) in a culture medium comprising: DZNep at a final concentration of 5 to 80 nM, preferably 5 to 50 nM or CPI-1205 at a final concentration of 0.5 to 5 mM, preferably DZNep at a final concentration of 5 to 15 nM or CPI-1205 at a final concentration of 0.5 to 2 mM, and optionally TSA at a final concentration of 3 to 10 nM, or VPA at a final concentration of 0.25 to 0.5 mM, or NaB at a final concentration of 0.25 to 0.5 mM, or DZNep at a final concentration of 5 to 15 nM or CPI-1205 at a final concentration of 0.5 to 2 mM and optionally TSA at a final concentration of 3 to 30 nM, or VPA at a final concentration of 0.25 to 2 mM, or NaB at a final concentration of 0.25 to 2 mM; L-ascorbic acid at a final concentration of 40 to 70 μg/mL; LIF at a final concentration of 10 to 30 ng/mL; PD0325901 at a final concentration of 0.5 to 1.5 μM; IWR1 or XAV939 at a final concentration of 3 to 6 μM; and wherein the culture medium is further supplemented with:
(1) ACTIVIN A or NODAL at a final concentration of 10 to 25 ng/mL; Y27632, thiazovivin or hydroxyfasudil at a final concentration in a range 0.5 to 2 μM; and an extracellular matrix in an amount of 0.1% to 0.5% (v/v); or
(2) ACTIVIN A or NODAL at a final concentration of 10 to 25 ng/mL; and Y27632, thiazovivin or hydroxyfasudil at a final concentration in a range 0.5 to 2 μM; or
(3) ACTIVIN A or NODAL at a final concentration of 10 to 25 ng/mL; and an extracellular matrix in an amount of 0.1% to 0.5% (v/v); or
(4) Y27632, thiazovivin or hydroxyfasudil at a final concentration of 0.5 to 2 μM; and an extracellular matrix in an amount of 0.1% to 0.5% (v/v); or
(5) ACTIVIN A or NODAL at a final concentration of 10 to 25 ng/mL; or Y27632, thiazovivin or hydroxyfasudil at a final concentration of 0.5 to 2 μM; or an extracellular matrix in an amount of 0.1% to 0.5% (v/v);
preferably the culture medium comprises: 10 nM DZNep or 1 mM CPI-1205; 50 μg/mL L-ascorbic acid; 20 ng/mL LIF; 1 μM PD0325901; 5 μM IWR1 or 5 μM XAV939; and optionally 5 nM TSA, or 0.5 mM VPA, or 0.5 mM NaB; and wherein the culture medium is further supplemented with (1) 20 ng/mL of ACTIVIN A or NODAL, 1 μM of Y27632, thiazovivin or hydroxyfasudil, and 0.2% (v/v) of an extracellular matrix; or (2) 20 ng/mL of ACTIVIN A or NODAL, and 1 μM of Y27632, thiazovivin or hydroxyfasudil; (3) 20 ng/mL of ACTIVIN A or NODAL, and 0.2% (v/v) of an extracellular matrix; or (4) 1 μM of Y27632, thiazovivin or hydroxyfasudil, and 0.2% (v/v) of an extracellular matrix; or (5) 20 ng/mL of ACTIVIN A or NODAL, or 1 μM of Y27632, thiazovivin or hydroxyfasudil, or 0.2% (v/v) of an extracellular matrix,
wherein the basal medium of the culture medium is selected from a group consisting of Dulbecco's modified eagle's medium (DMEM), minimal essential medium (MEM), basal medium Eagle (BME), RPMI1640, F10, F12, α minimal essential medium (a MEM), Glasgow's minimal essential medium (GMEM), Iscove's modified Dulbecco's medium, Neurobasal Medium and DMEM/F12, and a combination thereof; preferably, the basal medium is a mixture of Advanced DMEM/F12 and Neurobasal Medium in a ratio of 1:1 (v/v).

22. The method according to claim 13, wherein converting primate PSCs or ICLCs to 8CLCs comprises:
(a) genetically engineering the primate PSCs or ICLCs to reduce the activity of HDAC of the PSCs or ICLCs by knockdown or knockout of one or more relevant genes in the cells;
(b) culturing the genetically engineered cells in a culture medium comprising: DZNep at a final concentration of 40 to 70 nM or CPI-1205 at a final concentration of 2 to 4 mM; L-ascorbic acid at a final concentration of 40 to 70 μg/mL; LIF at a final concentration of 10 to 30 ng/mL; PD0325901 at a final concentration of 0.5 to 1.5 μM; IWR1 or XAV939 each at a final concentration of 3 to 6 μM; and optionally TSA at a final concentration of 10 to 30 nM, or VPA at a final concentration of 0.5 to 1.5 mM or NaB at a final concentration of 0.5 to 1.5 mM; and wherein the culture medium is further supplemented with:
(1) ACTIVIN A or NODAL at a final concentration of 10 to 25 ng/mL; Y27632, thiazovivin or hydroxyfasudil at a final concentration in a range 0.5 to 2 μM; and an extracellular matrix in an amount of 0.1% to 0.5% (v/v); or
(2) ACTIVIN A or NODAL at a final concentration of 10 to 25 ng/mL; and Y27632, thiazovivin or hydroxyfasudil at a final concentration of 0.5 to 2 μM; or
(3) ACTIVIN A or NODAL at a final concentration of 10 to 25 ng/mL; and an extracellular matrix in an amount of 0.1% to 0.5% (v/v); or
(4) Y27632, thiazovivin or hydroxyfasudil at a final concentration of 0.5 to 2 μM; and an extracellular matrix in an amount of 0.1% to 0.5% (v/v); or
(5) ACTIVIN A or NODAL at a final concentration of 10 to 25 ng/mL; or Y27632, thiazovivin or hydroxyfasudil at a final concentration in a range 0.5 to 2 μM; or an extracellular matrix in an amount of 0.1% to 0.5% (v/v);
preferably, the culture medium comprises: 50 nM DZNep or 3 mM CPI-1205; 50 μg/mL L-ascorbic acid; 20 ng/mL LIF; 1 μM PD0325901; 5 μM IWR1 or 5 μM XAV939; and optionally 20 nM TSA, or 1 mM VPA, or 1 mM NaB; and wherein the culture medium is further supplemented with (1) 20 ng/mL of ACTIVIN A or NODAL, 1 μM of Y27632, thiazovivin or hydroxyfasudil, and 0.2% (v/v) of an extracellular matrix; or (2) 20 ng/mL of ACTIVIN A or NODAL, and 1 μM of Y27632, thiazovivin or hydroxyfasudil; (3) 20 ng/mL of ACTIVIN A or NODAL, and 0.2% (v/v) of an extracellular matrix; or (4) 1 μM of Y27632, thiazovivin or hydroxyfasudil, and 0.2% (v/v) of an extracellular matrix; or (5) 20 ng/mL of ACTIVIN A or NODAL, or 1 μM of Y27632, thiazovivin or hydroxyfasudil, or 0.2% (v/v) of an extracellular matrix,
wherein the basal medium of the culture medium is selected from a group consisting of Dulbecco's modified eagle's medium (DMEM), minimal essential medium (MEM), basal medium Eagle (BME), RPMI1640, F10, F12, α minimal essential medium (a MEM), Glasgow's minimal essential medium (GMEM), Iscove's modified Dulbecco's medium, Neurobasal Medium, DMEM/F12 and Advanced DMEM/F12, and a combination thereof; preferably, the basal medium is a mixture of Advanced DMEM/F12 and Neurobasal Medium in a ratio of 1:1 (v/v).

23. The method according to claim 13, wherein the primate PSCs are selected from a group consisting of:
(i) cells from an ESC line and/or an ECC line;
(ii) cells from an iPSC line;
(iii) cells from ICM of a preimplantation blastocyst cultured in vitro;
(iv) cells from ICM of a post-implantation blastocyst cultured in vitro;
(v) cells from an embryo of 8C stage to morula stage cultured in vitro.

24. (canceled)

25. The method according to claim 13, wherein the method further comprises a step of culturing somatic cells in the presence of the SAH/PRC/EZH2 inhibitor and the HDAC inhibitor to reprogram the somatic cells to produce the primate ICLCs.

26. The method according to claim 13, comprising obtaining:
an isolated primate ICLC, wherein the PSC has transcriptome, transposable element profile, DNA methylome, chromatin landscape, and metabolic state close to a corresponding primate preimplantation ICM; or
an isolated primate 8CLC expressing 8C embryo specific markers at a level substantially higher than ICLCs and/or primed PSCs from which the 8CLC is produced; preferably, the cells have transcriptome, transposable element profile and chromatin landscape close a corresponding primate 8C stage embryo; preferably.

27-31. (canceled)

32. A kit comprising a SAH/PRC/EZH2 inhibitor and a HDAC inhibitor, and optionally
(1) one or more components selected from a group consisting of L-ascorbic acid, an activator of JAK/STAT3 signaling, an inhibitor of MAPK/ERK signaling, and a tankyrase inhibitor;
(2) one or more components selected from a group consisting of an activator of ACTIVIN/NODAL signaling, a ROCK inhibitor, and an extracellular matrix;
(3) one or more components selected from a group consisting of basal culture medium, serum replacement, alternative carbon source, non-essential amino acid, L-glutamine or its alternative and antibiotic.

33. (canceled)

34. A composition comprising a SAH/PRC/EZH2 inhibitor and a HDAC inhibitor, and optionally
(1) one or more components selected from a group consisting of L-ascorbic acid, an activator of JAK/STAT3 signaling, an inhibitor of MAPK/ERK signaling, and a tankyrase inhibitor; and
(2) one or more components selected from a group consisting of an activator of ACTIVIN/NODAL signaling, a ROCK inhibitor, and an extracellular matrix.

35. The composition according to claim 34, wherein the composition comprises DZNep or CPI-1205, and TSA or VPA or NaB, and optional L-ascorbic acid, optional LIF, optional PD0325901 and optional IWR1 or XAV939; preferably, each of the components is present in an amount that allows the culture medium containing the composition to comprise: 5 to 15 nM, preferably 10 nM, of DZNep, or 0.5 to 2 mM, preferably 1 mM, of CPI-1205; 3 to 6 nM, preferably 5 nM, of TSA, or 0.25 to 1 mM, preferably 0.5 mM, of VPA, or 0.25 to 1 mM, preferably 0.5 mM, of NaB; and optionally 40 to 90 μg/mL, preferably 50 μg/mL, of L-ascorbic acid, optionally 10 to 30 ng/mL, preferably 20 ng/mL, of LIF, optionally 0.5 to 1.5 μM, preferably 1 μM, of PD0325901, optionally 3 to 6 μM, preferably 5 μM, of IWR1 or XAV939; preferably, the compositions further comprise ACTIVIN A or NODAL, and/or Y27632, thiazovivin or hydroxyfasudil, and/or an extracellular matrix, wherein each of the components is present in an amount that allows the culture medium containing the composition to comprise 10 to 25 ng/mL, preferably 20 ng/mL ACTIVIN A or NODAL, and/or 0.5 to 2 μM, preferably 1 μM, of Y27632, thiazovivin or hydroxyfasudil, and/or 0.1% to 0.5% (v/v) of an extracellular matrix; or
the composition comprises DZNep or CPI-1205, and TSA or VPA or NaB, and optional L-ascorbic acid, optional LIF, optional PD0325901 and optional IWR1 or XAV939; preferably, each of the components is present in an amount that allows the culture medium containing the composition to comprise: 40 to 70 nM, preferably 50 nM, of DZNep, or 2 to 4 mM, preferably 3 mM, of CPI-1205; 10 to 30 nM, preferably 20 nM, of TSA, or 0.5 to 1.5 mM, preferably 1 mM, of VPA, or 0.5 to 1.5 mM, preferably 1 mM, of NaB; and optionally 40 to 90 μg/mL, preferably μg/mL, of L-ascorbic acid, optionally 10 to 30 ng/mL, preferably 20 ng/mL, of LIF, optionally 0.5 to 1.5 μM, preferably 1 μM, of PD0325901, optionally 3 to 6 μM, preferably 5 μM, of IWR1 or XAV939; preferably, the compositions further comprises ACTIVIN A or NODAL, and/or Y27632, thiazovivin or hydroxyfasudil, and/or an extracellular matrix, wherein each of the components is present in an amount that allows the culture medium containing the composition to comprise 10 to 25 ng/mL, preferably 20 ng/mL ACTIVIN A or NODAL, and/or to 2 μM, preferably 1 μM, of Y27632, thiazovivin or hydroxyfasudil, and/or 0.1% to 0.5% (v/v) of an extracellular matrix.

36. A method for reprogramming somatic cells to ICLCs, promoting conversion of primate PSCs to ICLCs, or for promoting conversion of primate one or more of PSCs and ICLCs to 8CLCs, comprising:
promoting expression of STELLA or improving activity of STELLA by an agent, or
promoting expression of one or more of KHDC1L, TRIM60 and genes belonging to ETCHbox family including TPRX1 and ARGFX, or improving activity of one or more of KHDC1L, TRIM60, and proteins belonging to ETCHbox family including TPRX1 and ARGFX by an agent,
wherein the agent is an inhibitor of SAH/PRC/EZH2, which includes DZNep and CPI-1205.

37-39. (canceled)
